(19) United States
(12) Patent Application Publication
(10) Pub. No.: US 2024/0002352 A1
(43) Pub. Date: Jan. 4, 2024

(54) COMPOUND, LIGHT-EMITTING MATERIAL, DELAYED FLUORESCENCE MATERIAL, AND ORGANIC LIGHT-EMITTING ELEMENT

(71) Applicant: KYULUX, INC., Fukuoka-shi, Fukuoka (JP)

(72) Inventors: Umamahesh BALIJAPALLI, Fukuoka-shi, Fukuoka (JP); Ryo NAGATA, Fukuoka-shi, Fukuoka (JP); Youichi TSUCHIYA, Fukuoka-shi, Fukuoka (JP); Hajime NAKANOTANI, Fukuoka-shi, Fukuoka (JP); Chihaya ADACHI, Fukuoka-shi, Fukuoka (JP)

(21) Appl. No.: 18/251,430

(22) PCT Filed: Nov. 2, 2021
(86) PCT No.: PCT/JP2021/040349

§ 371 Date: May 2, 2023

(30) Foreign Application Priority Data
Nov. 4, 2020 (JP) 2020-184095

(51) Int. Cl. C07D 241/46; C07D 413/14; H10K 50/11; H10K 85/60; C09K 11/06

(52) U.S. Cl. CPC C07D 241/46; C07D 413/14; H10K 50/11; H10K 85/654; C09K 11/06; C09K 2211/1022

(57) ABSTRACT

A high-efficiency near-infrared emitting organic EL device may be provided, using a compound represented by the following general formula. At least one of R¹ to R⁴ is *-Ar-D, or R¹ and R², R² and R³, or R³ and R⁴ bond to each other to form an aromatic ring having D. D represents a donor group, and Ar represents an arylene group.

### TECHNICAL FIELD

The present invention relates to a compound useful as a light emitting material, and a light emitting device using the compound.

### BACKGROUND ART

The development of high-efficiency near-infrared (NIR) light sources required in applications such as night vision displays, optical communications, information protection devices, and healthcare devices has been accelerated. As such a near-infrared light source, an inorganic light emitting diode is practically used at present. On the other hand, organic electroluminescent devices (organic EL devices) have advantages such as super-flexibility, ultra-light weight, and surface luminescence, and are therefore considered as candidates for near-infrared light sources of the next generation. However, an organic EL device of near infrared light emission has a problem derived from the device structure such as unbalanced charge trapping and a problem derived from materials such as quenching due to aggregate formation of luminescent molecules and non-radiation deactivation of excitons due to molecular vibration, and it is considered difficult to obtain a device performance comparable to that of an organic EL element of visible light emission. Under the situation, in order to realize a high-efficiency near-infrared organic EL device, various organic compounds and organometallic compounds have been developed, and have been studied for the performance thereof as near-infrared light emitting materials.

Specifically, in an organic EL device, singlet excitons and triplet excitons of an organic light emitting material are directly generated at a ratio of 25/75 by carrier recombination, and therefore, for obtaining a high luminous efficiency, the material need to be such that the energy thereof can be used for light emission before the triplet excitons are non-radiatively attenuated. As materials that uses triplet energy for light emission, room temperature phosphorescent materials typified by platinum complexes and iridium complexes and thermally activated delayed fluorescent materials are known. Among these, it is reported that, in an organic EL device using a platinum complex, an external quantum efficiency of 24% was achieved at an emission wavelength of 740 nm, and an external quantum efficiency of 3.8% was obtained at an emission wavelength of 900 nm.

### CITATION LIST

### Non-Patent Literature

NPL 1: Adv. Mater. 2011, 23, 2367.
NPL 2: Adv. Optical Mater. 2016, 4, 2068.
NPL 3: J. Mater. Chem. C 2015, 3, 2792.
NPL 4: Adv. Mater. 2018, 30, 1706584.
NPL 5: Adv. Optical Mater. 2020, 2000154.

### SUMMARY OF INVENTION

### Technical Problem

As described above, for room-temperature phosphorescent materials, there have been found near-infrared light-emitting materials that exhibit relatively high efficiency. However, metal complexes to be room-temperature phosphorescent materials are expensive as containing noble metal elements, and additionally have a problem in that stable supply thereof is difficult to maintain. As opposed to these, thermally activated delayed fluorescent materials have an advantage that the main constituent elements thereof are a carbon atom, a hydrogen atom and a nitrogen atom that exist inexhaustibly, and therefore studies and development thereof have been actively promoted. However, though there have been proposed some thermally activated delayed fluorescent materials that exhibit near-infrared light emission, they could not attain sufficiently satisfactory performance at present (for example, see NPLs 1 to 5).

Accordingly, in order to solve such a problem of the related art, the present inventors have conducted investigations for the purpose of providing an organic EL element capable of emitting near infrared light with high efficiency.

### Solution to Problem

As a result of assiduous studies for solving the above-mentioned problem, the present inventors have found that a phenazine derivative having a structure with a donor group linking to the phenazine skeleton via a 7-conjugated system is useful as alight emitting material. With that, the inventors have reached a finding that, by using this phenazine derivative as a light emitting material, an organic EL device capable of emitting near infrared light with high efficiency may be realized. The present invention has been proposed on the basis of these findings, and specifically has the following constitution.

[1] A compound represented by the following general formula (1):

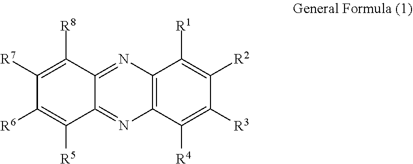

In the general formula (1), R¹ to R⁸ each independently represent a hydrogen atom or a substituent, R¹ and R², R² and R³, R³ and R⁴, R⁵ and R⁶, R⁶ and R⁷, and R⁷ and R⁸ each may bond to each other to form a cyclic structure, but do not form a heteroaryl ring. The general formula (1) satisfies at least one condition of the following (A) to (D).
(A) At least one of R¹ to R⁴ is *-Ar-D.
(B) R¹ and R² bond to each other to form an aromatic ring, and at least one *-Ar-D or D bonds to the aromatic ring.
(C) R² and R³ bond to each other to form an aromatic ring, and at least one *-Ar-D or D bonds to the aromatic ring.
(D) R³ and R⁴ bond to each other to form an aromatic ring, and at least one *-Ar-D or D bonds to the aromatic ring.

In the above (A) to (D), D represents a donor group, Ar represents an arylene group and * indicates a bonding position.

[2] The compound according to [1], in which two or more D's contained in the above (A) to (D) exist in a molecule of the compound.
[3] The compound according to [2], which satisfies the above (A) and in which at least two of R¹ to R⁴ are *-Ar-D.
[4] The compound according to [2], which satisfies the above (B) and (D).
[5] The compound according to any one of [2] to [4], in which D's existing in the molecule all have the same structure.
[6] The compound according to any one of [1] to [5], in which the general formula (1) satisfies at least one condition of the following (E) to (H).
(E) At least one of R⁵ to R⁸ is *-Ar-A.
(F) R⁵ and R⁶ bond to each other to form an aromatic ring, and at least one *-Ar-A or A bonds to the aromatic ring.
(G) R⁶ and R⁷ bond to each other to form an aromatic ring, and at least one *-Ar-A or A bonds to the aromatic ring.
(H) R⁷ and R⁸ bond to each other to form an aromatic ring, and at least one *-Ar-A or A bonds to the aromatic ring.

In the above (E) to (H), A represents an acceptor group, Ar represents an arylene group and * indicates a bonding position.

[7] The compound according to [1], which is represented by the following general formula (2):

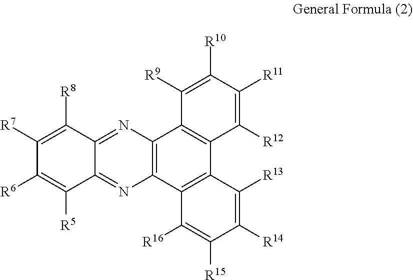

In the general formula (2), R⁵ to R¹⁶ each independently represent a hydrogen atom or a substituent, R⁵ and R⁶, R⁶ and R⁷, R⁷ and R⁸, R⁹ and R¹⁰, R¹⁰ and R¹¹, R¹¹ and R¹², R¹² and R¹³, R¹³ and R¹⁴, R¹⁴ and R¹⁵, and R¹⁵ and R¹⁶ each may bond to each other to form a cyclic structure, but do not form a heteroaryl ring. At least one of R⁹ to R¹⁶ is *-Ar-D or D. D represents a donor group, Ar represents an arylene group, and * indicates a bonding position.

[8] The compound according to [7], in which at least one of R⁶ and R⁷ is *-Ar-A or A, A represents an acceptor group, Ar represents an arylene group, and * indicates a bonding position.
[9] The compound according to [1], which is represented by the following general formula (3):

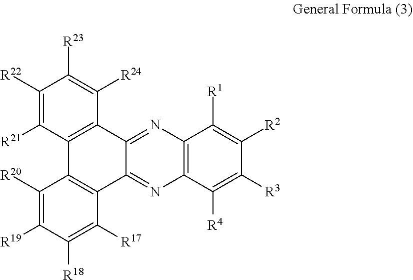

In the general formula (3), R¹ to R⁴ and R¹⁷ to R²⁴ each independently represent a hydrogen atom or a substituent, R¹ and R², R² and R³, R³ and R⁴, R¹⁷ and R¹⁸, R¹⁸ and R¹⁹, R¹⁹ and R²⁰, R²⁰ and R²¹, R²¹ and R²², R²² and R²³, and R²³ and R²⁴ each may bond to each other to form a cyclic structure. The general formula (3) satisfies at least one condition of the following (A) to (D).
(A) At least one of R¹ to R⁴ is *-Ar-D.
(B) R¹ and R² bond to each other to form an aromatic ring, and at least one *-Ar-D or D bonds to the aromatic ring.
(C) R² and R³ bond to each other to form an aromatic ring, and at least one *-Ar-D or D bond to the aromatic ring.
(D) R³ and R⁴ bond to each other to form an aromatic ring, and at least one *-Ar-D or D bonds to the aromatic ring.

In the above (A) to (D), D represents a donor group, Ar represents an arylene group and * indicates a bonding position.

The compound according to [1], which is represented by the following general formula (4):

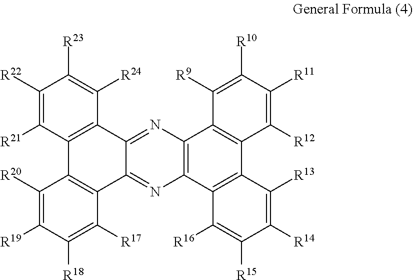

In the general formula (4), R⁹ to R²⁴ each independently represent a hydrogen atom or a substituent, R⁹ and R¹⁰, R¹⁰ and R¹¹, R¹¹ and R¹², R¹² and R¹³, R¹³ and R¹⁴, R¹⁴ and R¹⁵, R¹⁵ and R¹⁶, R¹⁷ and R¹⁸, R¹⁸ and R¹⁹, R¹⁹ and R²⁰, R²⁰ and R²¹, R²¹ and R²², R²² and R²³, and R²³ and R²⁴ each may bond to each other to form a cyclic structure. At least one of R⁹ to R¹⁶ is *-Ar-D or D. D represents a donor group, Ar represents an arylene group, and * indicates a bonding position.

The compound according to [9] or [10], in which at least one of R¹⁷ to R²⁴ is *-Ar-A or A, A represents an acceptor group, and * indicates a bonding position.

The compound according to [9] or [10], in which at least one of R¹⁹ and R²² is *-Ar-A or A, A represents an acceptor group, and * indicates a bonding position.

The compound according to any one of [7], and [10] to [12], in which at least one of R¹¹ and R¹⁴ is *-Ar-D or D.

A light emitting material of the compound of any one of [1] to [13].

A delayed fluorescent material of the compound of any one of [1] to [13].

An organic light emitting device containing the compound of any one of [1] to [13].

The organic light emitting device according to [16], containing the compound in the light emitting layer.

The organic light emitting device according to [17], in which the light emitting layer contains a light emitting material, and among the light emission from the organic light emitting device, the amount of light emission from the light emitting material is the maximum.

The organic light emitting device according to [17], in which the light emitting layer contains a host material.

The organic light emitting device according to any one of [16] to [19], having an emission peak wavelength at 590 to 990 nm.

### Advantageous Effects of Invention

The compound of the present invention is useful as a light emitting material. Also, the compound of the present invention includes a compound that emits delayed fluorescence. The organic light emitting device using the compound of the present invention as a light emitting material may realize high-efficiency near-infrared light emission.

### BRIEF DESCRIPTION OF DRAWING

FIG. 1 This is a schematic cross-sectional view showing an example of a layer configuration of an organic electroluminescent device.

FIG. 2 This shows emission spectra of various thin films produced by changing the concentration of Compound 1.

### DESCRIPTION OF EMBODIMENTS

The present invention will be described in detail below. The constitutional elements may be described below with reference to representative embodiments and specific examples of the invention, but the invention is not limited to such embodiments. In the description herein, a numerical range expressed as “to” means a range that includes the numerical values described before and after “to” as the lower limit and the upper limit. The hydrogen atom that is present in the molecule of the compound used in the invention is not particularly limited in isotope species, and for example, all the hydrogen atoms in the molecule may be ¹H, and all or a part of them may be ²H (deuterium D). In the present description, “near-infrared light” means a light whose wavelength falls within a range of 680 to 2500 nm.

### [Compound Represented by General Formula (1)]

The compound of the present invention is a compound represented by the following general formula (1).

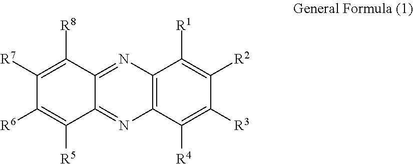

In the general formula (1), R¹ to R⁸ each independently represent a hydrogen atom or a substituent. R¹ to R⁸ may be the same as or different from each other. R¹ and R², R² and R³, R³ and R⁴, R⁵ and R⁶, R⁶ and R⁷, and R⁷ and R⁸ each may bond to each other to form a cyclic structure, but do not form a heteroaryl ring.

With that, the general formula (1) satisfies at least one condition of the following (A) to (D).
(A) At least one of R¹ to R⁴ is *-Ar-D.
(B) R¹ and R² bond to each other to form an aromatic ring, and at least one *-Ar-D or D bonds to the aromatic ring.
(C) R² and R³ bond to each other to form an aromatic ring, and at least one *-Ar-D or D bonds to the aromatic ring.
(D) R³ and R⁴ bond to each other to form an aromatic ring, and at least one *-Ar-D or D bonds to the aromatic ring.

In the above (A) to (D), D represents a donor group, Ar represents an arylene group and * indicates a bonding position.

The donor group that D represents means a substituent which may readily donate an electron to the side of the bonding atom, as compared with a hydrogen. The donor group is preferably a substituent having a negative Hammett's σₚ value.

Here, “Hammett's σₚ value” is one propounded by L. P. Hammett, and is one to quantify the influence of a substituent on the reaction rate or the equilibrium of a para-substituted benzene derivative. Specifically, the value is a constant (σₚ value) peculiar to the substituent in the following equation that is established between a substituent and a reaction rate constant or an equilibrium constant in a para-substituted benzene derivative:

log(k/k₀)=ρσₚ

or

log(K/K₀)=ρσₚ

In the above equations, k represents a rate constant of a benzene derivative not having a substituent; k₀ represents a rate constant of a benzene derivative substituted with a substituent; K represents an equilibrium constant of a benzene derivative not having a substituent; K₀ represents an equilibrium constant of a benzene derivative substituted with a substituent; ρ represents a reaction constant to be determined by the kind and the condition of reaction. Regarding the description relating to the “Hammett's σₚ value” and the numerical value of each substituent in the present description, reference may be made to the description relating to σₚ value in Hansch, C. et. al., Chem. Rev., 91, 165-195 (1991).

The donor group that D represents includes a diarylamino group, and a polycyclic condensed heterocyclic group having a structure such that the aryl groups of a diarylamino group bond to each other via a single bond or via a linking group. The aromatic ring of the aryl group in the diarylamino group may be a monocyclic ring or a condensed ring of two or more aromatic rings condensed together. The carbon number of the aromatic ring is preferably 6 to 40, more preferably 6 to 22, even more preferably 6 to 18, further more preferably 6 to 14, particularly preferably 6 to 10. Specific examples of the aryl group include a phenyl group and a naphthalenyl group. The aryl group may be substituted with a substituent. Regarding the preferred range and the specific examples of the substituent, reference can be made to the preferred range and the specific examples of the substituent that the following R⁶¹ to R⁷⁰ may have. Also regarding the description, the preferred range and the specific examples of the linking group that links aryl groups, reference can be made to the description relating to the linking group that links R⁶⁵ and R⁶⁶ mentioned below.

Preferred examples of the donor group include groups represented by the following general formula (5).

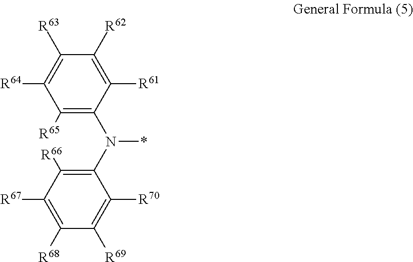

In the general formula (5), R⁶¹ to R⁷⁰ each independently represent a hydrogen atom or a substituent. R⁶¹ to R⁷⁰ may be the same as or different from each other. * indicates a bonding position.

Examples of the substituent that R⁶¹ to R⁷⁰ may have include a hydroxy group, a halogen atom, an alkyl group having 1 to 20 carbon atoms, an alkoxy group having 1 to 20 carbon atoms, an alkylthio group having 1 to 20 carbon atoms, an alkyl-substituted amino group having 1 to 20 carbon atoms, an aryl-substituted amino group having 1 to 20 carbon atoms, an aryl group having 6 to 40 carbon atoms, a heteroaryl group having 3 to 40 carbon atoms, an alkenyl group having 2 to 10 carbon atoms, an alkynyl group having 2 to 10 carbon atoms, an alkylamide group having 2 to 20 carbon atoms, an arylamide group having 7 to 21 carbon atoms, and a trialkylsilyl group having 3 to 20 carbon atoms. Of those specific groups, these capable of being further substituted with a substituent may be substituted. More preferred substituents are an alkyl group having 1 to 20 carbon atoms, an alkoxy group having 1 to 20 carbon atoms, an alkylthio group having 1 to 20 carbon atoms, an alkyl-substituted amino group having 1 to 20 carbon atoms, an aryl-substituted amino group having 1 to 20 carbon atoms, an aryl group having 6 to 40 carbon atoms, and a heteroaryl group having 3 to 40 carbon atoms.

R⁶¹ and R⁶², R⁶² and R⁶³, R⁶³ and R⁶⁴, R⁶⁴ and R⁶⁵, R⁶⁵ and R⁶⁶, R⁶⁶ and R⁶⁷, R⁶⁷ and R⁶⁸, R⁶⁸ and R⁶⁹, and R⁶⁹ and R⁷⁰ each may bond to each other to form a cyclic structure. The cyclic structure may be an aromatic ring or an aliphatic ring, or may contain a hetero atom, and further the cyclic structure may be a condensed ring of two or more rings. The hetero atom as referred to herein is preferably one selected from the group consisting of a nitrogen atom, an oxygen atom and a sulfur atom. Examples of the cyclic structure to be formed include a benzene ring, a naphthalene ring, a pyridine ring, a pyridazine ring, a pyrimidine ring, a pyrazine ring, a pyrrole ring, an imidazole ring, a pyrazole ring, a triazole ring, an imidazoline ring, an oxazole ring, an isoxazole ring, a thiazole ring, an isothiazole ring, a cyclohexadiene ring, a cyclohexene ring, a cyclopentaene ring, a cycloheptatriene ring, a cycloheptadiene ring, and a cycloheptaene ring.

Of the groups represented by the general formula (5), preferred are R⁶⁵ and R⁶⁶ not bonding to each other, R⁶⁵ and R⁶⁶ bonding to each other to form a single bond, or R⁶⁵ and R⁶⁶ bonding to each other to form a linking group whose linking chain length is one atom. In the case where R⁶⁵ and R⁶⁶ bond to each other to form a linking group whose linking chain length is one atom, the cyclic structure to be formed as a result of bonding of R⁶⁵ and R⁶⁶ to each other is a 6-membered ring. Specific examples of the linking group to be formed by R⁶⁵ and R⁶⁶ bonding to each other include linking groups represented by —O—, —S—, —N(R¹⁶¹)— or —C(R¹⁶²)(R¹⁶³)—. Here, R¹⁶¹ to R¹⁶³ each independently represent a hydrogen atom or a substituent. Examples of the substituent that R¹⁶¹ may have include an alkyl group having 1 to 20 carbon atoms, an aryl group having 6 to 40 carbon atoms, and a heteroaryl group having 3 to 40 carbon atoms. The substituent that R¹⁶² and R¹⁶³ may have each is independently a hydroxy group, a halogen atom, an alkyl group having 1 to 20 carbon atoms, an alkoxy group having 1 to 20 carbon atoms, an alkylthio group having 1 to 20 carbon atoms, an alkyl-substituted amino group having 1 to 20 carbon atoms, an aryl-substituted amino group having 12 to 40 carbon atoms, an aryl group having 6 to 40 carbon atoms, a heteroaryl group having 3 to 40 carbon atoms, an alkenyl group having 2 to 10 carbon atoms, an alkynyl group having 2 to 10 carbon atoms, an alkylamide group having 2 to 20 carbon atoms, an arylamide group having 7 to 21 carbon atoms, or a trialkylsilyl group having 3 to 20 carbon atoms.

Preferred examples of the group represented by the general formula (5) include groups represented by any of the following general formulae (6) to (10).

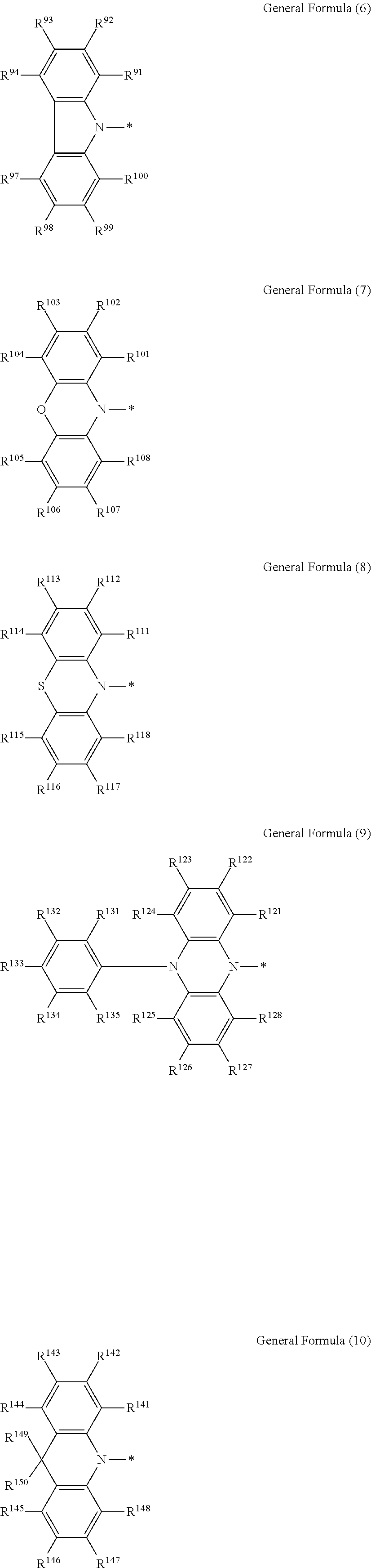

In the general formulae (6) to (10), R⁹¹ to R⁹⁴, R⁹⁷ to R¹⁰⁸, R¹¹¹ to R¹¹⁸, R¹²¹ to R¹²⁸, R¹³¹ to R¹³⁵, and R¹⁴¹ to R¹⁵⁰ each independently represent a hydrogen atom or a substituent. R⁹¹ and R⁹², R⁹² and R⁹³, R⁹³ and R⁹⁴, R⁹⁷ and R⁹⁸, R⁹⁸ and R⁹⁹, R⁹⁹ and R¹⁰⁰, R¹⁰¹ and R¹⁰², R¹⁰² and R¹⁰³, R¹⁰³ and R¹⁰⁴, R¹⁰⁵ and R¹⁰⁶, R¹⁰⁶ and R¹⁰⁷, R¹⁰⁷ and R¹⁰⁸, R¹¹¹ and R¹¹², R¹¹² and R¹¹³, R¹¹³ and R¹¹⁴, R¹¹⁵ and R¹¹⁶, R¹¹⁶ and R¹¹⁷, R¹¹⁷ and R¹¹⁸, R¹²¹ and R¹², R¹²² and R¹²³, R¹²³ and R¹²⁴, R¹²⁵ and R¹²⁶, R¹²⁶ and R¹²⁷, R¹²⁷ and R¹²⁸, R¹³¹ and R¹³², R¹³² and R¹³³, R¹³³ and R¹³⁴, R¹³⁴ and R¹³⁵, R¹²⁴ and R¹³¹, R¹²⁵ and R¹³⁵, R¹⁴¹ and R¹⁴², R¹⁴² and R¹⁴³, R¹⁴³ and R¹⁴⁴, R¹⁴⁵ and R¹⁴⁶, R¹⁴⁶ and R¹⁴⁷, R¹⁴⁷ and R¹⁴⁸, and R¹⁴⁹ and R¹⁵⁰ each may bond to each other to form a cyclic structure. * indicates a bonding position.

Here, regarding the description, the preferred range and the specific examples of the substituent and the cyclic structure, reference can be made to the description, the preferred range and the specific examples of the substituent and the cyclic structure in the general formula (5).

In (A) to (D) in the formula (1), Ar in *-Ar-D represents an arylene group. The aromatic ring constituting the arylene group may be a monocyclic ring, or a condensed ring formed by condensation of two or more aromatic rings, or a linked ring of two or more aromatic rings linking to each other. In the case where two or more aromatic rings link, they may link linearly, or may rink in a branched manner. The carbon number of the aromatic ring constituting the arylene group is preferably 6 to 40, more preferably 6 to 22, even more preferably 6 to 18, further more preferably 6 to 14, further more preferably 6 to 10. Specific examples of the arylene group include a phenylene group, a naphthalenediyl group, and a biphenyldiyl group, and a phenylene group is preferred. The phenylene group may be any of a 1,2-phenylene group, a 1,3-phenylene group, or a 1,4-phenylene group, but is preferably a 1,4-phenylene group. The hydrogen atom of the arylene group may be substituted with a substituent. Regarding the preferred range and the specific examples of the substituent, reference can be made to the preferred range and the specific examples of the substituent that the above-mentioned R⁶¹ to R⁷⁰ may have.

In (B) to (D), the aromatic ring formed by R¹ and R² bonding to each other, the aromatic ring formed by R² and R³ bonding to each other, and the aromatic ring formed by R³ and R⁴ bonding to each other each may be a monocyclic ring or a condensed ring formed by condensation of two or more aromatic rings. The carbon number of the aromatic ring is preferably 6 to 24, more preferably 6 to 18, even more preferably 6 to 14. Specific examples of the aromatic ring include a benzene ring and a naphthalene ring. The hydrogen atom of the aromatic ring may be substituted with a substituent. Regarding the preferred range and the specific examples of the substituent, reference can be made to the preferred range and the specific examples of the substituent that the above-mentioned R⁶¹ to R⁷⁰ may have.

The condition that the general formula (1) satisfies may be one or more of (A) to (D). The compound represented by the general formula (1) may contain in the molecule one alone or two or more aromatic rings formed by D and Ar in (A) to (D), and R¹ and R², R² and R³, or R³ and R⁴ in (B) to (D) bonding to each other. When the formula contains two or more of these in the molecule, the plural D's, the plural Ar's and the plural aromatic rings may be the same as or different from each other. Here, preferably, two or more D's in (A) to (D) exist in the molecule, and preferably, D's existing in the molecule all have the same structure.

When the general formula (1) satisfies (A), one alone or two or more of R¹ to R⁴ may be *-Ar-D, but preferably at least two of R¹ to R⁴ are *-Ar-D, more preferably at least R² and R³ are *-Ar-D, even more preferably at least R¹ and R⁴ are *-Ar-D. When two or more of R¹ to R⁴ are *-Ar-D, the plural (*-Ar-D)'s may be the same as or different from each other.

When the general formula (1) satisfies at least one of (B) to (D), *-Ar-D or D bonding to each aromatic ring may be one in one aromatic ring or two or more in one aromatic ring. When two or more (*-Ar-D)'s or D's bond to one aromatic ring, the plural (*-Ar-D)'s and the plural D's each may be the same as or different from each other.

Preferably, the general formula (1) satisfies (A) where at least two of R¹ to R⁴ are *-Ar-D, or satisfies both (B) and (D).

Further preferably, the general formula (1) satisfies at least one condition of the following (E) to (H).
(E) At least one of R⁵ to R⁸ is *-Ar-A.
(F) R⁵ and R⁶ bond to each other to form an aromatic ring, and at least one *-Ar-A or A bonds to the aromatic ring.
(G) R⁶ and R⁷ bond to each other to form an aromatic ring, and at least one *-Ar-A or A bonds to the aromatic ring.
(H) R⁷ and R⁸ bond to each other to form an aromatic ring, and at least one *-Ar-A or A bonds to the aromatic ring.

In the above (E) to (H), A represents an acceptor group, Ar represents an arylene group and * indicates a bonding position.

The acceptor group that A represents means a substituent which may readily accept an electron from the side of the bonding atom, as compared with a hydrogen. Preferably, the acceptor group is a substituent having a positive Hammett's σₚ value.

Examples of the acceptor group that A represents include a cyano group, a halogen atom, a halogenoalkyl group and a nitro group. Examples of the halogen atom, and the halogen atom in the halogenoalkyl group include a fluorine atom, a chlorine atom, a bromine atom and an iodine atom. The carbon number of the halogenoalkyl group is preferably 1 to 20, more preferably 1 to 10, even more preferably 1 to 6.

The acceptor group that A represents also includes the group represented by the following general formula (11).

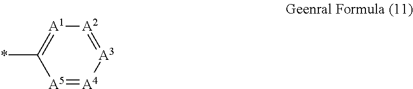

In the general formula (11), A¹ to A⁵ each independently represent N or C(R¹⁶⁴), R¹⁶⁴ represents a hydrogen atom or a substituent. Preferably, at least one of A¹ to A⁵ N, and 1 to 3 thereof are N. When the group represented by the general formula (11) has plural R¹⁶⁴'s, the plural R¹⁶⁴'s may be the same as or different from each other. * indicates a bonding position. Examples of the substituent that R¹⁶⁴ may have include an alkyl group having 1 to 20 carbon atoms, an aryl group having 6 to 40 carbon atoms, a cyano group, a halogen atom, and a heteroaryl group having 5 to 40 carbon atoms. Preferred is an aryl group having 6 to 40 carbon atoms. Of these substituents, those capable of being substituted with a substituent may be substituted.

Specific examples of the group represented by the general formula (11) include a pyridyl group, a pyrimidyl group, a pyridazyl group, a pyrazyl group, and a triazinyl group. These groups may be substituted with a substituent. Regarding the preferred range and the specific examples of the substituent, reference can be made to the preferred range and the specific examples of the substituent that R¹⁶⁴ may have.

In (E) to (H), regarding the description, the preferred range and the specific examples of the arylene group that Ar represents and the substituent with which the arylene group may be substituted, reference can be made to the description relating to Ar in (A) to (D) mentioned above. However, when Ar represents a phenylene group, it is preferably a 1,4-phenylene group and is also preferably a 1,3-phenylene group. Regarding the description, the preferred range and the specific examples of the aromatic ring formed by R⁸ and R⁶ in (F) to (H) bonding to each other, the aromatic ring formed by R⁶ and R⁷ bonding to each other, and the aromatic ring formed by R⁷ and R⁸ bonding to each other, reference can be made to the description relating to the aromatic ring formed by R¹ and the like in (B) to (D) mentioned above bonding to each other.

The condition that the general formula (1) satisfies may be one alone or two or more of (E) to (H). The compound represented by the general formula (1) may contain in the molecule, one alone or two or more aromatic rings formed by A and Ar in (E) to (H), or R⁵ and R⁶, R⁶ and R⁷, or R⁷ and R⁸ in (F) to (H) bonding to each other. When two or more of these exist in the molecule, the plural aromatic rings of the plural A's and the plural Ar's may be the same as or different from each other.

When the general formula (1) satisfies (E), one or two or more of R⁵ to R⁸ may be *-Ar-A. Preferably, at least one of R⁶ and R⁷ is *-Ar-A. When two or more of R⁵ to R⁸ are *-Ar-A, the plural (*-Ar-A)′ may be the same as or different from each other.

When the general formula (1) satisfies at least one of (F) to (H), *-Ar-A or A bonding to each aromatic ring may be one in one aromatic ring or two or more in one aromatic ring. When two or more (*-Ar-A)′ or A's bond to one aromatic ring, the plural (*-Ar-A)'s and the plural A's each may be the same as or different from each other.

Preferably, the general formula (1) satisfies (E), or satisfies both (F) and (H).

Of R¹ to R⁸ in the general formula (1), the remaining ones excluding those contained in (A) to (H) may be a hydrogen atom or a substituent, or the neighboring substituents may form a cyclic structure (but excluding a heteroaryl ring). Regarding the preferred range and the specific examples of the substituent, reference can be made to the preferred range and the specific examples of the substituent that R⁶¹ to R⁷⁰ may have (but excluding those corresponding to *-Ar-D and *-Ar-A). The cyclic structure that the neighboring substituents together form may be an aromatic ring or an aliphatic ring, or may be one containing a hetero atom (but excluding a heteroaryl group), and further, the cyclic structure may be a condensed ring of two or more rings. The hetero atom as referred to herein is preferably one selected from the group consisting of a nitrogen atom, an oxygen atom and a sulfur atom. Examples of the cyclic structure to be formed include a benzene ring, a naphthalene ring, an imidazoline ring, a cyclohexadiene ring, a cyclohexene ring, a cyclopentaene ring, a cycloheptatriene ring, a cycloheptadiene ring, and a cycloheptaene ring.

Examples of the compound represented by the general formula (1) include compounds represented by the following general formula (2).

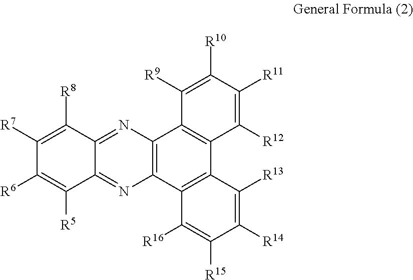

In the general formula (2), R⁵ to R¹⁶ each independently represent a hydrogen atom or a substituent, R⁵ to R¹⁶ may be the same as or different from each other. R⁵ and R⁶, R⁶ and R⁷, R⁷ and R⁸, R⁹ and R¹⁰, R¹⁰ and R¹¹, R¹¹ and R¹², R¹² and R¹³, R¹³ and R¹⁴, R¹⁴ and R¹⁵, and R¹⁵ and R¹⁶ each may bond to each other to form a cyclic structure, but do not form a heteroaryl ring. At least one of R⁹ to R¹⁶ is *-Ar-D or D. D represents a donor group, Ar represents an arylene group, * indicates a bonding position.

Regarding the description, the preferred range and the specific examples of *-Ar-D and D that at least one of R⁹ to R¹⁶ represents, reference may be made to the description relating to *-Ar-D and D in the general formula (1) mentioned above. Of R⁹ to R¹⁶, *-Ar-D or D is preferably at least one of R¹¹ and R.

At least one of R⁶ and R⁷ is preferably *-Ar-A or A, more preferably *-Ar-A. Here, A represents an acceptor group, Ar represents an arylene group and * indicates a bonding position. Regarding the description, the preferred range and the specific examples of *-Ar-A and A, reference can be made to the description relating to *-Ar-A and A in the general formula (1) mentioned above.

Those excluding R⁹ to R¹⁶ which are *-Ar-D or D may be a hydrogen atom, or may be a substituent (but excluding those corresponding to *-Ar-D or D), or of those, the neighboring substituents may form a cyclic structure (but excluding a heteroaryl ring). Those excluding R⁵ to R⁸ which are *-Ar-A may be a hydrogen atom, or may be a substituent (but excluding those corresponding to *-Ar-A), or of those, the neighboring substituents may form a cyclic structure (but excluding a heteroaryl ring). Regarding the description of the substituent, and the description and the specific examples of the cyclic structure formed by the neighboring substituents, reference can be made to the description relating to the substituent and the cyclic structure that, of R¹ to R⁸ in the above general formula (1), the remaining ones excluding those contained in (A) to (H) may have.

Examples of the compound represented by the general formula (1) also include compounds represented by the following general formula (3).

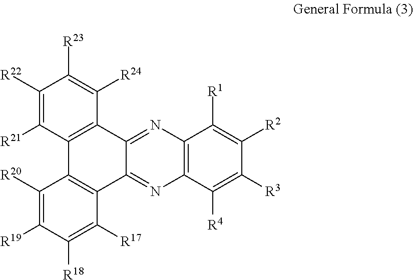

In the general formula (3), R¹ to R⁴ and R¹⁷ to R²⁴ each independently represent a hydrogen atom or a substituent, R¹ to R⁴ and R¹⁷ to R²⁴ may be the same as or different from each other. R¹ and R², R² and R³, R³ and R⁴, R¹⁷ and R¹⁸, R¹⁸ and R¹⁹, R¹⁹ and R²⁰, R²⁰ and R²¹, R²¹ and R²², R²² and R²³, and R²³ and R²⁴ each may bond to each other to form a cyclic structure. The general formula (3) satisfies at least one condition of the following (A) to (D).
(A) At least one of R¹ to R⁴ is *-Ar-D.
(B) R¹ and R² bond to each other to form an aromatic ring, and at least one *-Ar-D or D bonds to the aromatic ring.
(C) R² and R³ bond to each other to form an aromatic ring, and at least one *-Ar-D or D bond to the aromatic ring.
(D) R³ and R⁴ bond to each other to form an aromatic ring, and at least one *-Ar-D or D bonds to the aromatic ring.

In the above (A) to (D), D represents a donor group, Ar represents an arylene group and * indicates a bonding position.

Regarding the description of (A) to (D), reference can be made to the description relating to (A) to (D) in the general formula (1) mentioned above.

In the general formula (3), preferably at least one of R¹⁷ to R²⁴ is *-Ar-A or A. Here, A represents an acceptor group, Ar represents an arylene group, and * indicates a bonding position. Regarding the description, the preferred range and the specific examples of *-Ar-A or A, reference can be made to the description relating to *-Ar-A or A in the general formula (1) mentioned above. Of R¹⁷ to R²⁴, preferably at least one of R¹⁸, R¹⁹, R²² and R²³ is *-Ar-A or A, more preferably at least one of R¹⁸ and R¹⁹ and at least one of R²² and R²³ are *-Ar-A or A, even more preferably at least one of R¹⁹ and R²³ is *-Ar-A or A.

The remaining R¹ to R⁴ except those contained in (A) to (D) may be a hydrogen atom, or may be a substituent (but excluding those corresponding to *-Ar-D), or the neighboring substituents may form a cyclic structure (but excluding a heteroaryl ring). R¹⁷ to R²⁴ of the remainder excluding those which are *-Ar-A or A may be a hydrogen atom, or may be a substituent (but excluding those corresponding to *-Ar-A and A), or the neighboring substituents may form a cyclic structure (but excluding a heteroaryl ring). Regarding the description of the substituent, and the description and the specific examples of the cyclic structure to be formed by the neighboring substituents, reference can be made to the description relating to the substituent and the cyclic structure that the remaining R¹ to R⁸ in the general formula (1) except those contained in (A) to (H) may have.

The compound represented by the general formula (1) is also preferably a compound represented by the following general formula (4).

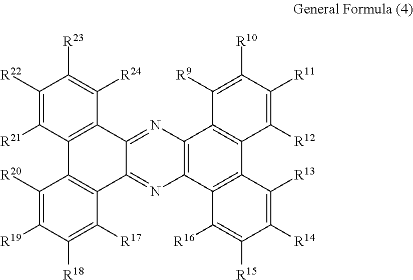

In the general formula (4), R⁹ to R²⁴ each independently represent a hydrogen atom or a substituent, R⁹ and R¹⁰, R¹⁰ and R¹¹, R¹¹ and R¹², R¹² and R¹³, R¹³ and R¹⁴, R¹⁴ and R¹⁵, R¹⁵ and R¹⁶, R¹⁷ and R¹⁸, R¹⁸ and R¹⁹, R¹⁹ and R²⁰, R²⁰ and R²¹, R²¹ and R²², R²² and R²³, and R²³ and R²⁴ each may bond to each other to form a cyclic structure. At least one of R⁹ to R¹⁶ is *-Ar-D or D. D represents a donor group, Ar represents an arylene group, and * indicates a bonding position.

Regarding the description of R⁹ to R¹⁶, reference can be made to the description relating to R⁹ to R¹⁶ in the general formula (2) mentioned above, and regarding the description of R¹⁷ to R²⁴, reference can be made to the description relating to R¹⁷ to R²⁴ in the general formula (3) mentioned above.

In the following, specific examples of the compound represented by the general formula (1) are shown. However, the compound represented by the general formula (1) that can be used in the present invention should not be limitatively interpreted by these specific examples. In the following formulae, Me represents a methyl group.

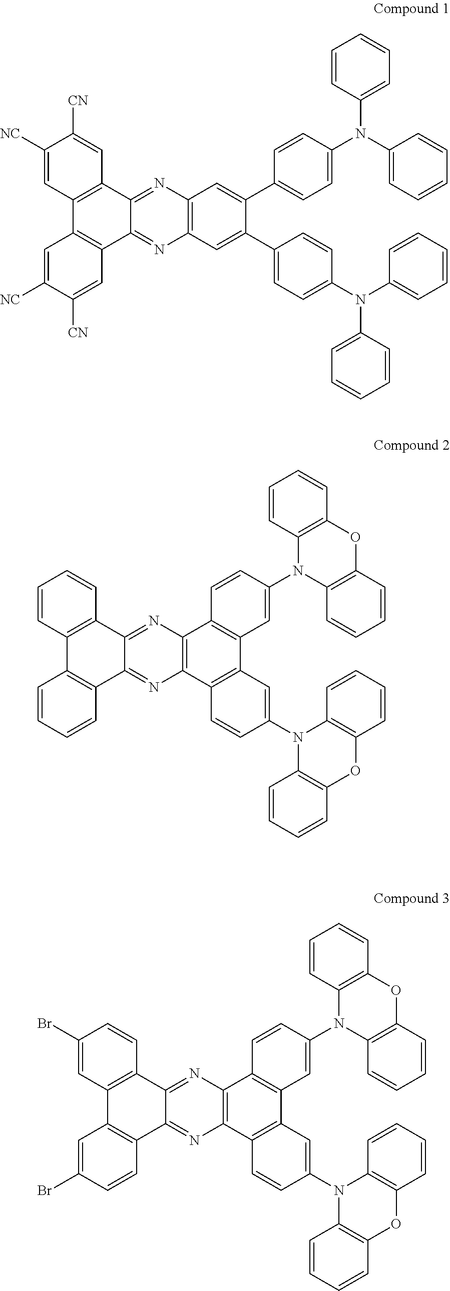

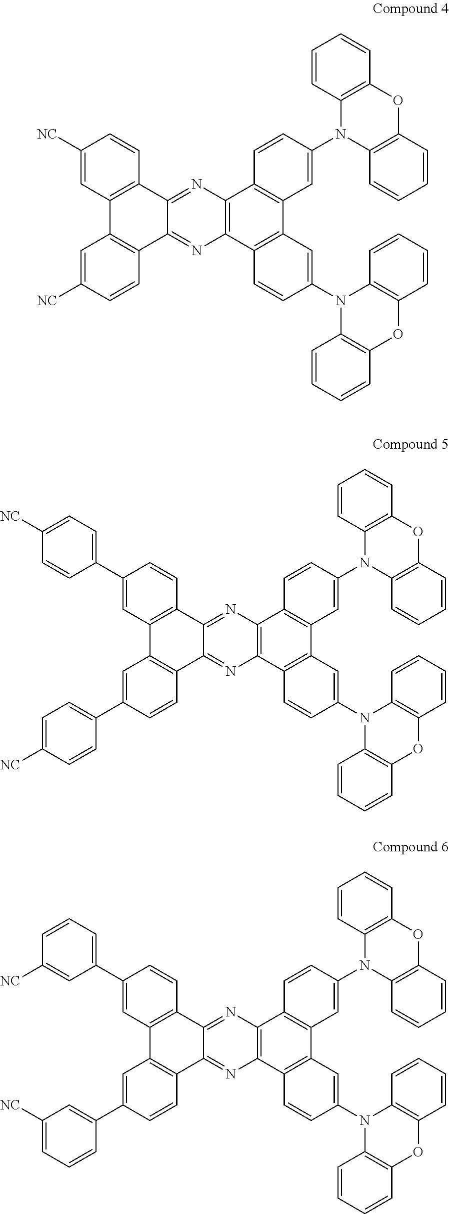

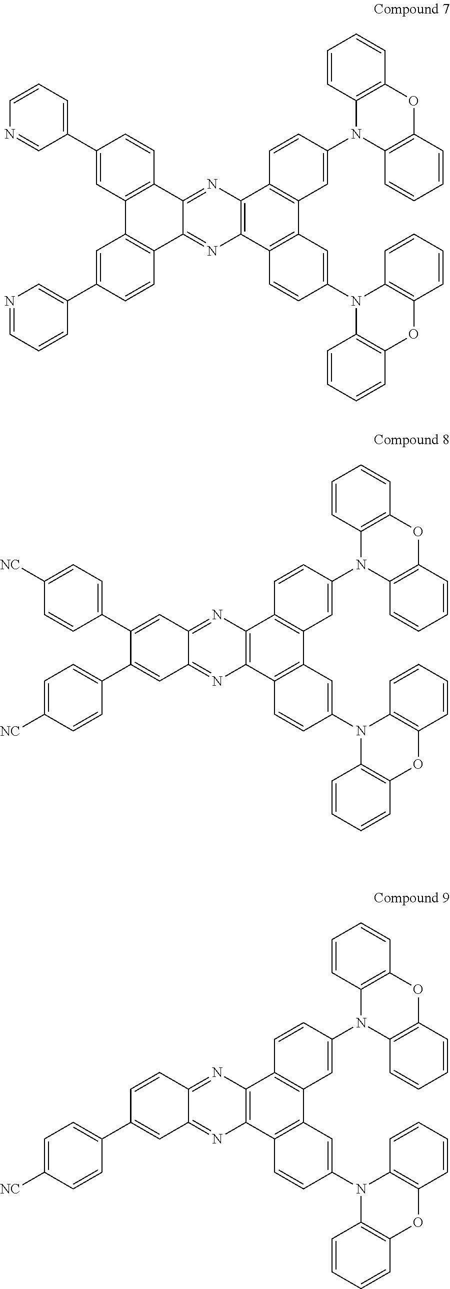

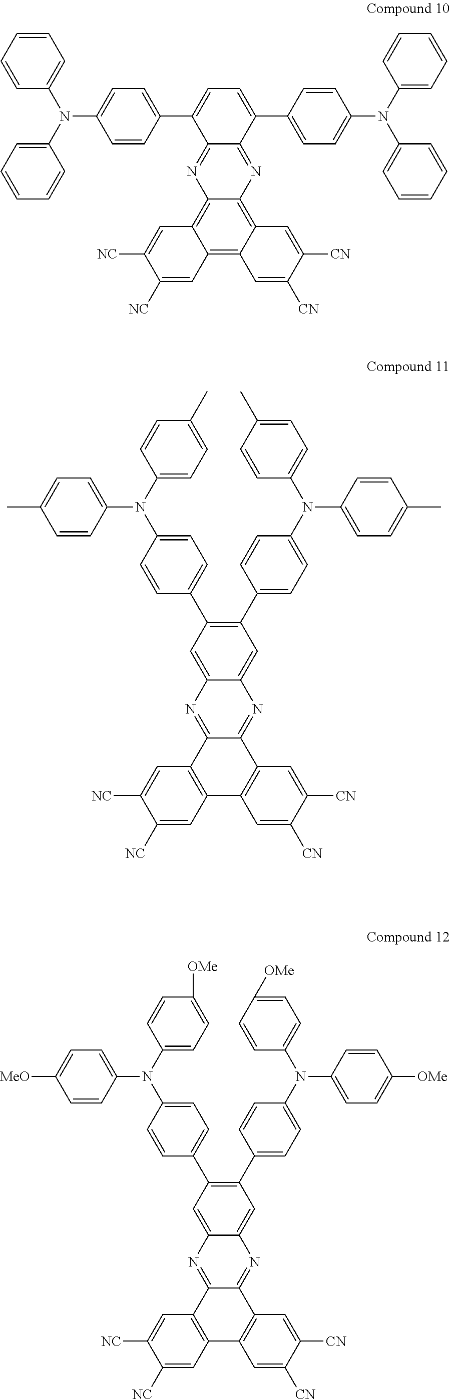

### [Synthesis Method for Compound Represented by General Formula (1)]

The compound represented by the general formula (1) can be synthesized by combining known reactions. For example, of the compound represented by the general formula (1), the compound represented by the general formula (3) where at least one of R¹ to R⁴ is *-Ar-D, and R¹⁸, R¹⁹, R²² and R²³ are *-Ar-A or A can be synthesized by reacting the following two compounds to obtain an intermediate A, and then replacing X¹ and X² with -Ar-A or A. For example, a compound where R¹⁸, R¹⁹, R²² and R²³ are cyano groups can be synthesized by reaction of the intermediate A and copper(I) cyanide.

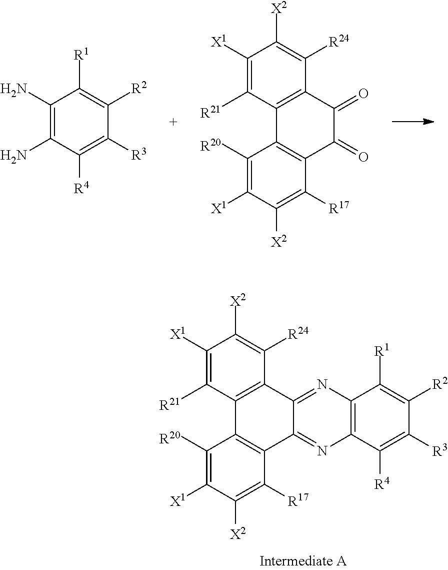

Regarding the description of R¹ to R⁴, R¹⁷, R²⁰, R²¹, and R²⁴ in the above reaction formula, reference can be made to the corresponding description in the general formula (3). X¹ and X² each represent a halogen atom, including a fluorine atom, a chlorine atom, a bromine atom and an iodine atom. X¹ is preferably a bromine atom, X² is preferably an iodine atom.

A known condensation reaction is applied to the above reaction, for which known reaction conditions can be appropriately selected and used. Regarding details of the above reaction and other synthesis methods for the compound represented by the general formula (1), reference can be made to Synthesis Examples given hereinunder. In addition, the compound represented by the general formula (1) can also be synthesized by combining any other known synthesis reactions.

### [Light Emitting Material and Delayed Fluorescent Material]

The light emitting material and the delayed fluorescent material of the present invention includes the compound of the present invention.

The compound of the present invention is the compound represented by the general formula (1), and regarding the description thereof, reference can be made to the description in the section of [Compound Represented by General Formula (1)].

The compound represented by the general formula (1) may emit near-infrared light at high efficiency, and is therefore useful as a light emitting material.

The reason why the compound represented by the general formula (1) has a high emission efficiency is presumed because the energy difference ΔE_{ST} between the excited singlet energy level E_{S1} and the excited triplet energy level E_{T1} is small and therefore reverse intersystem crossing from the excited triplet state to the excited singlet state readily occurs and the excited triplet energy may be effectively utilized for singlet exciton formation and light emission. The mechanism is described below.

The compound of the present invention is represented by the general formula (1), and has a structure where a donor group bonds to the phenazine skeleton having an acceptor property via an arylene group (condition (A)), or has a structure where a donor group directly bonds to the aromatic ring condensed with the phenazine skeleton, or a structure where a donor group bonds to the aromatic ring via an arylene group (conditions (B) to (D)). In other words, the structure of the compound is such that a donor group and an acceptor group (phenazine skeleton) link via an expanded 1 electron system (arylene group, aromatic ring). Accordingly, HOMO (highest occupied molecular orbital) localized in the donor group and LUMO (lowest unoccupied molecular orbital) localized in the acceptor group are separated spatially to lower the exchange interaction between HOMO-LUMO, and the energy difference ΔE_{ST} between the excited singlet energy level E_{S1} and the excited triplet energy level E_{T1} thereby lowers. Further, in the case where an acceptor group (A) is introduced into a predetermined site of the phenazine skeleton (conditions (E) to (H)), the acceptor property of the phenazine skeleton can be increased markedly, and the tendency of the energy state becomes remarkable.

Here, for example, when an organic compound is current-excited, singlet excitons and triplet excitons form in a ratio of 25/75, and in an ordinary organic compound, triplet excitons undergo non-radiation deactivation at room temperature and are not effectively utilized for light emission. Consequently, the energy of triplet excitons accounting for 75% of excitons runs to waste, and improvement of emission efficiency is limited. As opposed to this, in a compound whose ΔE_{ST} is small, reverse intersystem crossing readily occurs to the excited singlet state before triplet excitons undergo non-radiation deactivation, and light emission occurs by radiation deactivation from the excited singlet state. The compound represented by the general formula (1) has a small value of ΔE_{ST} owing to the molecular structure as above, and therefore it is presumed that, such a mechanism effectively works and the energy of triplet excitons may be efficiently utilized for light emission to attain a high emission efficiency. Further, since ΔE_{ST} is small, a value of the reverse intersystem crossing rate constant is large, and accordingly, accumulation of triplet excitons is suppressed in a high current region so that the roll-off phenomenon owing to triplet-triplet annihilation may be thereby suppressed. As a result, the effect of increasing the maximum external quantum efficiency may also be attained.

In the above-mentioned emission mechanism, the emission from the excited singlet state formed by reverse intersystem crossing is observed later than the fluorescence radiation from the excited singlet state directly formed by current excitation (instantaneous fluorescence), and is therefore referred to as “delayed fluorescence”. The emission lifetime of ordinary delayed fluorescence is 0.05 μs or more.

The compound represented by the general formula (1) has small ΔE_{ST}, and may radiate such delayed fluorescence efficiently, and is therefore highly useful as a delayed fluorescent material.

Further, some compounds represented by the general formula (1) are such that in a mixed film of the compound and a host compound, by varying the concentration of the compound, the emission wavelength from the mixed film changes. The emission wavelength of the thin film containing such a compound can be controlled by a simple method of changing the concentration of the compound in the thin film, and therefore the compounds of the type are more highly useful as a light emitting material and a delayed fluorescent material.

Some compounds represented by the general formula (1) hardly undergo aggregation of molecules. This is presumed because the molecular structure may readily have anon-planar twisted structure. Such compounds may prevent concentration quenching induced by aggregation of molecules, and are more highly useful as a light emitting material and a delayed fluorescent material.

### [Structure Using the Compound Represented by the General Formula (1)]

In some embodiments, the compound represented by the general formula (1) is used along with one or more materials (e.g., small molecules, polymers, metals, metal complexes), by combining them, or by dispersing the compound, or by covalent-bonding with the compound, or by coating with the compound, or by carrying the compound, or by associating with the compound, and solid films or layers are formed. For example, by combining the compound represented by the general formula (1) with an electroactive material, a film can be formed. In some cases, the compound represented by the general formula (1) may be combined with a hole transporting polymer. In some cases, the compound represented by the general formula (1) may be combined with an electron transporting polymer. In some cases, the compound represented by the general formula (1) may be combined with a hole transporting polymer and an electron transporting polymer. In some cases, the compound represented by the general formula (1) may be combined with a copolymer having both a hole transporting moiety and an electron transporting moiety. In the embodiments mentioned above, the electrons and/or the holes formed in a solid film or layer may be interacted with the compound represented by the general formula (1).

### [Film Formation]

In some embodiments, a film containing the compound represented by the general formula (1) may be formed in a wet process. In a wet process, a solution prepared by dissolving a composition containing the compound of the present invention is applied onto a surface, and then the solvent is removed to form a film. The wet process includes a spin coating method, a slit coating method, an ink jet method (a spraying method), a gravure printing method, an offset printing method and flexographic printing method, which, however are not limitative. In the wet process, an appropriate organic solvent capable of dissolving a composition containing the compound of the present invention is selected and used. In some embodiments, a substituent (e.g., an alkyl group) capable of increasing the solubility in an organic solvent may be introduced into the compound contained in the composition.

In some embodiments, a film containing the compound of the present invention may be formed in a dry process. In some embodiments, a vacuum evaporation method is employable as a dry process, which, however, is not limitative. In the case where a vacuum evaporation method is employed, compounds to constitute a film may be co-evaporated from individual evaporation sources, or may be co-evaporated from a single evaporation source formed by mixing the compounds. In the case where a single evaporation source is used, a mixed powder prepared by mixing compound powders may be used, or a compression molded body prepared by compressing the mixed powder may be used, or a mixture prepared by heating and melting the constituent compounds and cooling the resulting melt may be used. In some embodiments, by co-evaporation under the condition where the evaporation rate (weight reduction rate) of the plural compounds contained in a single evaporation source is the same or is nearly the same, a film having a compositional ratio corresponding to the compositional ratio of the plural compounds contained in the evaporation source may be formed. When plural compounds are mixed in the same compositional ratio as the compositional ratio of the film to be formed to prepare an evaporation source, a film having a desired compositional ratio can be formed in a simplified manner. In some embodiments, the temperature at which the compounds to be co-evaporated has the same weight reduction ratio is specifically defined, and the temperature can be employed as the temperature of co-evaporation.

### [Use Examples of Compound Represented by General Formula (1)]

### Organic Light Emitting Diode:

The organic light emitting device of the present invention is characterized by containing the compound of the present invention.

The compound of the present invention is the compound represented by the general formula (1), and regarding the description thereof, reference can be made to the description in the section of [Compound Represented by General Formula (1)].

The organic light emitting device containing the compound represented by the general formula (1) in the light emitting layer may realize high-efficiency near-infrared emission. By varying the concentration of the compound represented by the general formula (1) contained in the light emitting layer, the emission wavelength may also be controlled. For example, by varying the concentration, the emission peak wavelength may be controlled in a broad wavelength range of 590 to 990 nm.

One embodiment of the present invention relates to use of the compound represented by the general formula (1) of the present invention as a light emitting material for organic light emitting devices. In some embodiments, the compound represented by the general formula (1) of the present invention can be effectively used as a light emitting material in a light emitting layer in an organic light emitting device.

In some embodiments, the compound represented by the general formula (1) of the present invention includes delayed fluorescence that emits delayed fluorescence (delayed fluorescent material). In some embodiments, the present invention provides a delayed fluorescent material having a structure represented by the general formula (1) of the present invention. In some embodiments, the present invention relates to use of the compound represented by the general formula (1) of the present invention as a delayed fluorescent material. In some embodiments, the compound represented by the general formula (1) of the present invention can be used as a host material, and can be used along with one or more light-emitting materials, and the light emitting material can be a fluorescent material, a phosphorescent material or a TADF material (thermally activated delayed fluorescent material). In some embodiments, the compound represented by the general formula (1) can be used as a hole transporting material. In some embodiments, the compound represented by the general formula (1) can be used as an electron transporting material. In some embodiments, the present invention relates to a method of generating delayed fluorescence from the compound represented by the general formula (1). In some embodiments, the organic light emitting device containing the compound as a light emitting material emits delayed fluorescence and shows a high light emission efficiency.

In some embodiments, the light emitting layer contains the compound represented by the general formula (1), and the compound represented by the general formula (1) is aligned in parallel to the substrate. In some embodiments, the substrate is a film-forming surface. In some embodiment, the alignment of the compound represented by the general formula (1) relative to the film-forming surface can have some influence on the propagation direction of light emitted by the aligned compounds, or can determine the direction. In some embodiments, by aligning the propagation direction of light emitted by the compound represented by the general formula (1), the light extraction efficiency from the light emitting layer can be improved.

One embodiment of the present invention relates to an organic light emitting device. In some embodiments, the organic light emitting device includes a light emitting layer. In some embodiments, the light emitting layer contains, as a light emitting material therein, the compound represented by the general formula (1). In some embodiments, the organic light emitting device is an organic photoluminescent device (organic PL device). In some embodiments, the organic light emitting device is an organic electroluminescent device (organic EL device). In some embodiments, the compound represented by the general formula (1) assists light irradiation from the other light emitting materials contained in the light emitting layer (as a so-called assist dopant). In some embodiments, the compound represented by the general formula (1) contained in the light emitting layer is in a lowest excited energy level, and is contained between the lowest excited single energy level of the host material contained in the light emitting layer and the lowest excited singlet energy level of the other light emitting materials contained in the light emitting layer.

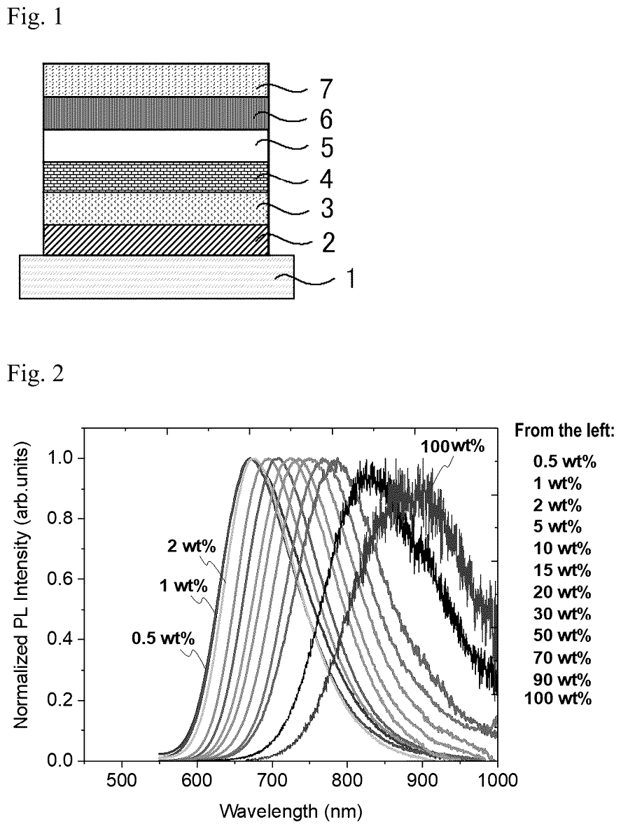

In some embodiments, the organic photoluminescent device comprises at least one light-emitting layer. In some embodiments, the organic electroluminescent device comprises at least an anode, a cathode, and an organic layer between the anode and the cathode. In some embodiments, the organic layer comprises at least a light-emitting layer. In some embodiments, the organic layer comprises only a light-emitting layer. In some embodiments, the organic layer comprises one or more organic layers in addition to the light-emitting layer. Examples of the organic layer include a hole transporting layer, a hole injection layer, an electron barrier layer, a hole barrier layer, an electron injection layer, an electron transporting layer and an exciton barrier layer. In some embodiments, the hole transporting layer may be a hole injection and transporting layer having a hole injection function, and the electron transporting layer may be an electron injection and transporting layer having an electron injection function. An example of an organic electroluminescent device is shown in FIG. 1.

### (Light Emitting Layer)

In some embodiments, the light emitting layer is a layer where holes and electrons injected from the anode and the cathode, respectively, are recombined to form excitons. In some embodiments, the layer emits light.

In some embodiments, only a light emitting material is used as the light emitting layer. In some embodiments, the light emitting layer contains a light emitting material and a host material. In some embodiments, the light emitting material is one or more compounds of the general formula (1). In some embodiments, for improving luminous radiation efficiency of an organic electroluminescent device and an organic photoluminescence device, the singlet exciton and the triplet exciton generated in a light emitting material is confined inside the light emitting material. In some embodiments, a host material is used in the light emitting layer in addition to a light emitting material therein. In some embodiments, the host material is an organic compound. In some embodiments, the organic compound has an excited singlet energy and an excited triplet energy, and at least one of them is higher than those in the light emitting material of the present invention. In some embodiments, the singlet exciton and the triplet exciton generated in the light emitting material of the present invention are confined in the molecules of the light emitting material of the present invention. In some embodiments, the singlet and triplet excitons are fully confined for improving luminous radiation efficiency. In some embodiments, although high luminous radiation efficiency is still attained, singlet excitons and triplet excitons are not fully confined, that is, a host material capable of attaining high luminous radiation efficiency can be used in the present invention with no specific limitation. In some embodiments, in the light emitting material in the light emitting layer of the device of the present invention, luminous radiation occurs. In some embodiments, radiated light includes both fluorescence and delayed fluorescence. In some embodiments, radiated light includes radiated light from a host material. In some embodiments, radiated light is composed of radiated light from a host material. In some embodiments, radiated light includes radiated light from the compound represented by the general formula (1) and radiated light from a host material. In some embodiment, a TADF molecule and a host material are used. In some embodiments, TADF is an assist dopant.

In some embodiments where a host material is used, the amount of the compound of the present invention as the light emitting material contained in the light emitting layer is 0.10% by weight or more. In some embodiments where a host material is used, the amount of the compound of the present invention contained in the light emitting layer is 1% by weight or more. In some embodiments where a host material is used, the amount of the compound of the present invention as the light emitting material contained in the light emitting layer is 50% by weight or less. In some embodiments where a host material is used, the amount of the compound of the present invention as the light emitting material contained in the light emitting layer is 20% by weight or less. In some embodiments where a host material is used, the amount of the compound of the present invention as the light emitting material contained in the light emitting layer is 10% by weight or less.

In some embodiments, the host material in the light emitting layer is an organic compound having a hole transporting function and an electron transporting function. In some embodiments, the host material in the light emitting layer is an organic compound that prevents increase in the wavelength of radiated light. In some embodiments, the host material in the light emitting layer is an organic compound having a high glass transition temperature.

In some embodiments, the host material is selected from the following group:

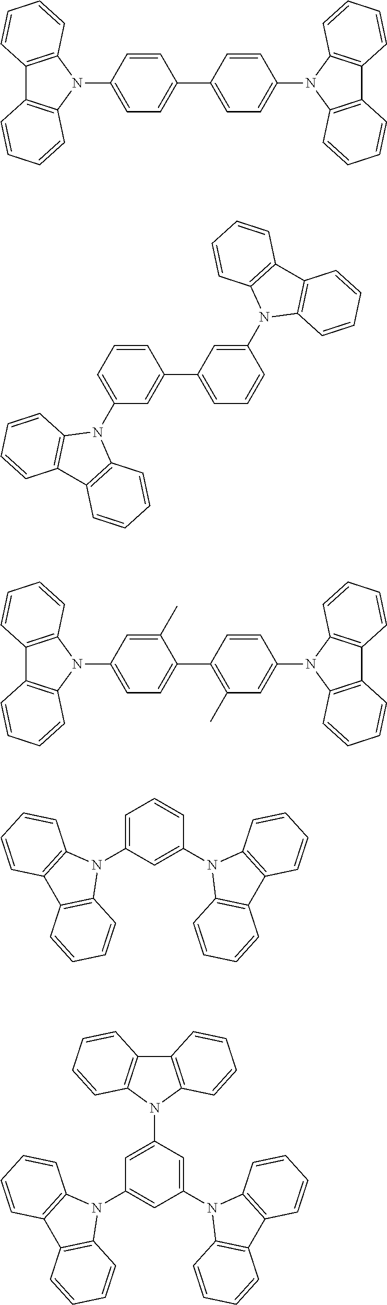 [SMILES: c1ccc2c(c1)c1ccccc1n2-c1ccc(-c2ccc(-n3c4ccccc4c4ccccc43)cc2)cc1 ; c1cc(-c2cccc(-n3c4ccccc4c4ccccc43)c2)cc(-n2c3ccccc3c3ccccc32)c1 ; Cc1cc(-n2c3ccccc3c3ccccc32)ccc1-c1ccc(-n2c3ccccc3c3ccccc32)cc1C ; c1cc(-n2c3ccccc3c3ccccc32)cc(-n2c3ccccc3c3ccccc32)c1 ; c1ccc2c(c1)c1ccccc1n2-c1cc(-n2c3ccccc3c3ccccc32)cc(-n2c3ccccc3c3ccccc32)c1]

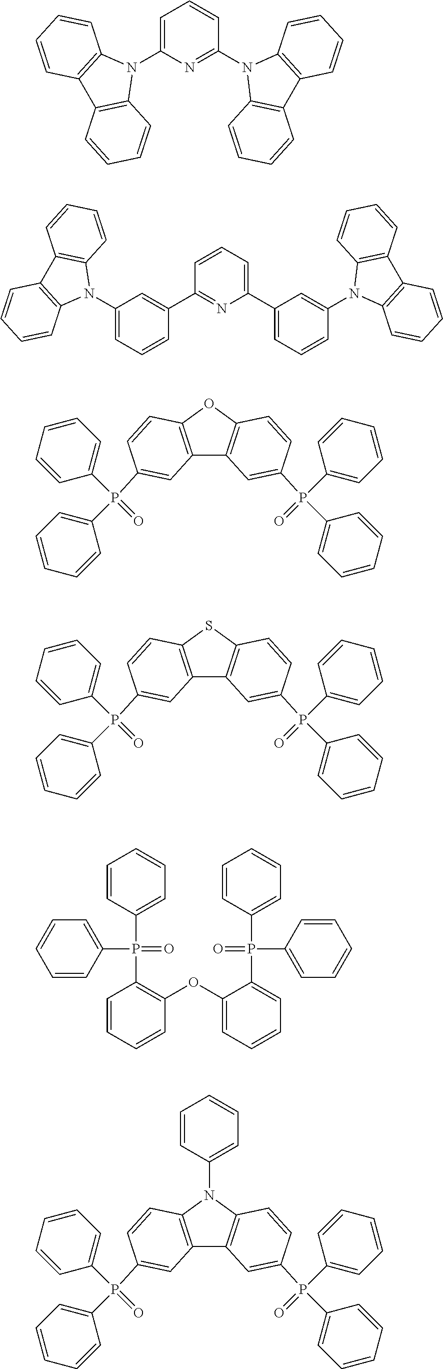 [SMILES: c1cc(-n2c3ccccc3c3ccccc32)nc(-n2c3ccccc3c3ccccc32)c1 ; c1cc(-c2cccc(-c3cccc(-n4c5ccccc5c5ccccc54)c3)n2)cc(-n2c3ccccc3c3ccccc32)c1 ; O=P(c1ccccc1)(c1ccccc1)c1ccc2oc3ccc(P(=O)(c4ccccc4)c4ccccc4)cc3c2c1 ; O=P(c1ccccc1)(c1ccccc1)c1ccc2sc3ccc(P(=O)(c4ccccc4)c4ccccc4)cc3c2c1 ; O=P(c1ccccc1)(c1ccccc1)c1ccccc1Oc1ccccc1P(=O)(c1ccccc1)c1ccccc1 ; O=P(c1ccccc1)(c1ccccc1)c1ccc2c(c1)c1cc(P(=O)(c3ccccc3)c3ccccc3)ccc1n2-c1ccccc1]

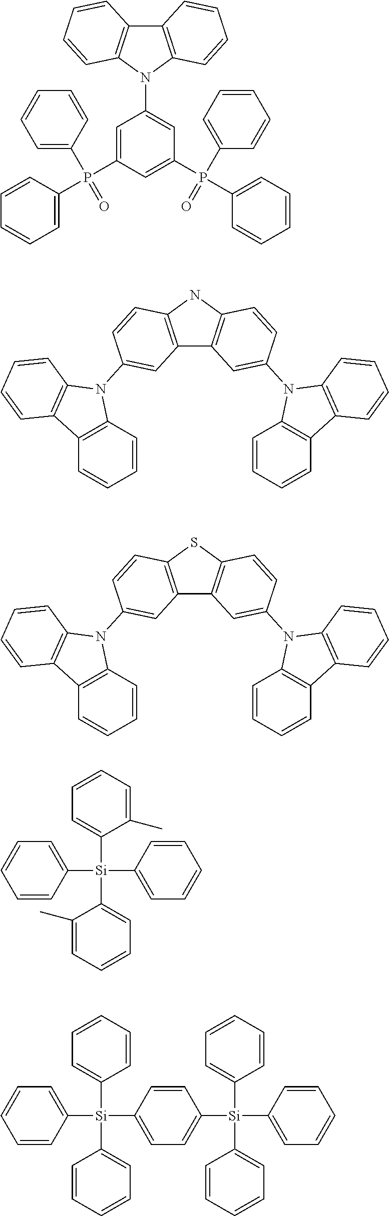

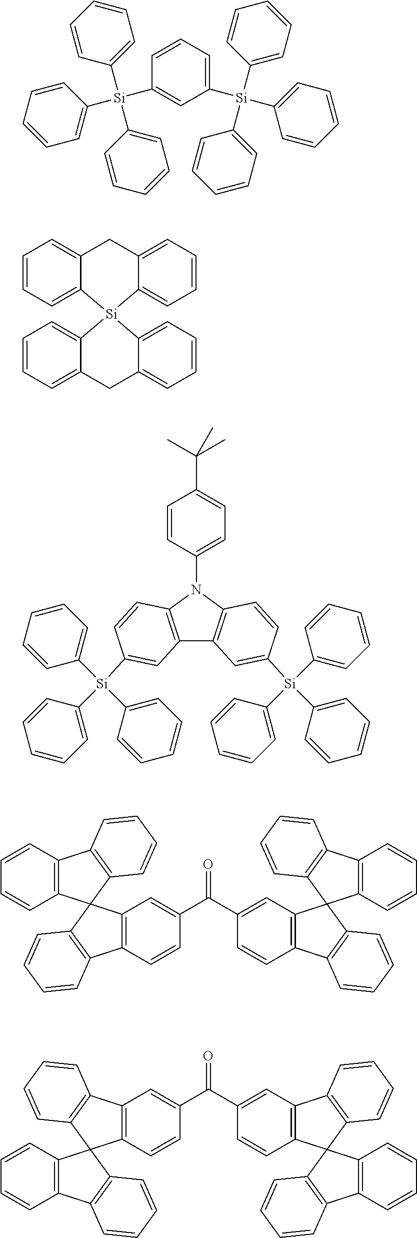 [SMILES: c1ccc([Si](c2ccccc2)(c2ccccc2)c2cccc([Si](c3ccccc3)(c3ccccc3)c3ccccc3)c2)cc1 ; c1ccc2c(c1)Cc1ccccc1[Si]21c2ccccc2Cc2ccccc21 ; CC(C)(C)c1ccc(-n2c3ccc([Si](c4ccccc4)(c4ccccc4)c4ccccc4)cc3c3cc([Si](c4ccccc4)(c4ccccc4)c4ccccc4)ccc32)cc1 ; O=C(c1ccc2c(c1)C1(c3ccccc3-c3ccccc31)c1ccccc1-2)c1ccc2c(c1)C1(c3ccccc3-c3ccccc31)c1ccccc1-2 ; O=C(c1ccc2c(c1)-c1ccccc1C21c2ccccc2-c2ccccc21)c1ccc2c(c1)-c1ccccc1C21c2ccccc2-c2ccccc21]

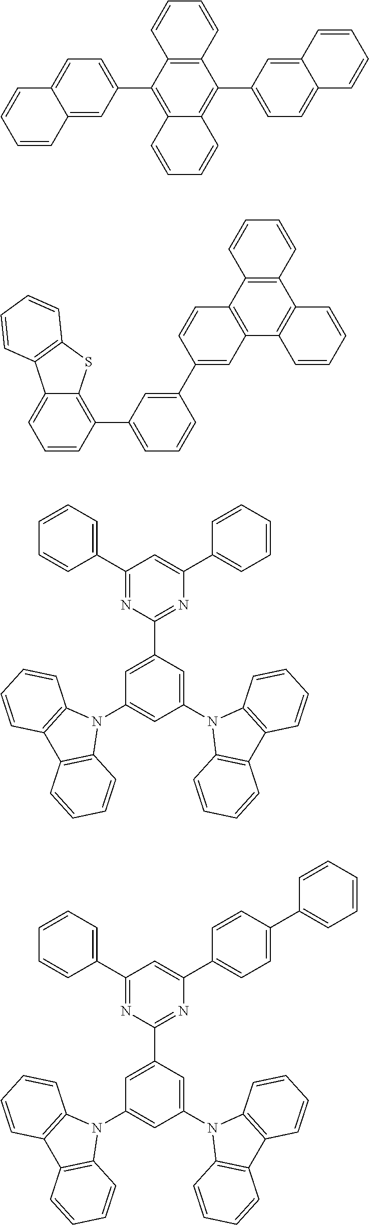

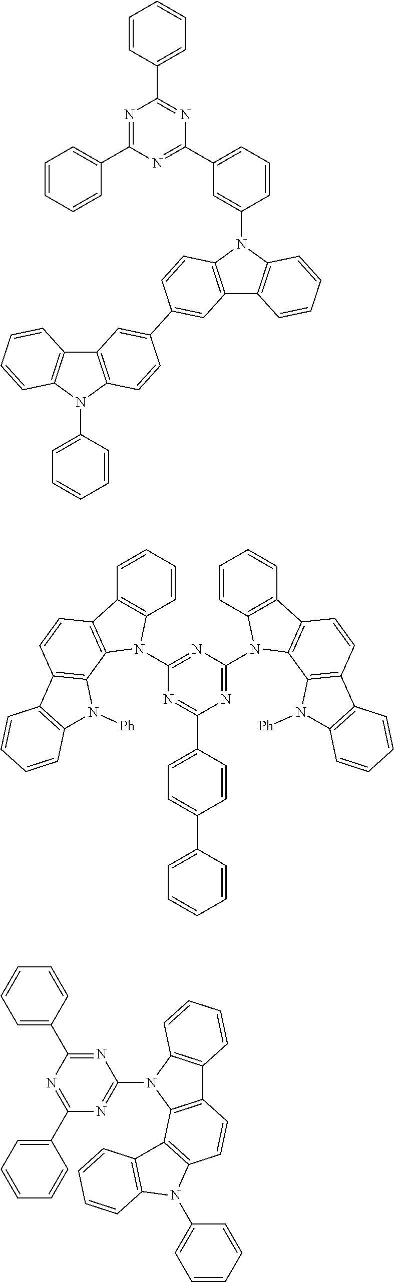 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1]

In some embodiments, the light emitting layer contains at least two TADF molecules differing in the structure. For example, the light emitting layer can contain three kinds of materials, a host material, a first TADF molecule and a second TADF molecule whose excited singlet energy level is higher in that order. At that time, the first TADF molecule and the second TADF molecule are preferably such that the difference ΔE_{ST} between the lowest excited singlet energy level and the lowest excited triplet energy level at 77 K thereof is 0.3 eV or less, more preferably 0.25 eV or less, even more preferably 0.2 eV or less, further more preferably 0.15 eV or less, further more preferably 0.1 eV or less, further more preferably 0.07 eV or less, further more preferably 0.05 eV or less, further more preferably 0.03 eV or less, especially more preferably 0.01 eV or less. The content of the first TADF molecule in the light emitting layer is preferably larger than the content of the second TADF molecule therein. The content of the host material in the light emitting layer is preferably larger than the content of the second TADF molecule therein. The content of the first TADF molecule in the light emitting layer can be larger than the content of the host material therein, or can be smaller than or the same as the latter. In some embodiments, the composition in the light emitting layer can be 10 to 70% by weight of the host material, 10 to 80% by weight of the first TADF molecule, and 0.1 to 30% by weight of the second TADF molecule. In some embodiments, the composition in the light emitting layer can be 20 to 45% by weight of the host material, 50 to 75% by weight of the first TADF molecule, and 5 to 20% by weight of the second TADF molecule. In some embodiments, the photoluminescence quantum yield φPL1(A) by photoexcitation of the co-deposited film of the first TADF molecule and the host material (the content of the first TADF molecule in the co-deposited film=A % by weight) and the photoluminescence quantum yield φPL2(A) by photoexcitation of the co-deposited film of the second TADF molecule and the host material (the content of the second TADF molecule in the co-deposited film=A % by weight) satisfy a relational formula φPL1(A)>φPL2(A). In some embodiments, the photoluminescence quantum yield φPL2(B) by photoexcitation of the co-deposited film of the second TADF molecule and the host material (the content of the second TADF molecule in the co-deposited film=B % by weight) and the photoluminescence quantum yield φPL2(100) by photoexcitation of the single film of the second TADF molecule satisfy a relational formula φPL2(B)>φPL2(100). In some embodiments, the light emitting layer can contain three TADF molecules differing in the structure. The compound in the present invention can be any of plural TADF compounds contained in the light emitting layer.

In some embodiments, the light emitting layer does not contain a metal element. In some embodiments, the light emitting layer can be composed of a material composed of atoms alone selected from the group consisting of a carbon atom, a hydrogen atom, a nitrogen atom, an oxygen atom and a sulfur atom. Or the light emitting layer can be composed of a material composed of atoms alone selected from the group consisting of a carbon atom, a hydrogen atom, a nitrogen atom and an oxygen atom.

When the light emitting layer contains any other TADF material than the compound of the present invention, the TADF material can be a known delayed fluorescent material. Preferred delayed fluorescent materials are compounds included in the general formulae described in WO2013/154064, paragraphs 0008 to 0048 and 0095 to 0133; WO2013/011954, paragraphs 0007 to 0047 and 0073 to 0085; WO2013/011955, paragraphs 0007 to 0033 and 0059 to 0066; WO2013/081088, paragraphs 0008 to 0071 and 0118 to 0133; JP 2013-256490 A, paragraphs 0009 to 0046 and 0093 to 0134; JP 2013-116975 A, paragraphs 0008 to 0020 and 0038 to 0040; WO2013/133359, paragraphs 0007 to 0032 and 0079 to 0084; WO2013/161437, paragraphs 0008 to 0054 and 0101 to 0121; JP 2014-9352 A, paragraphs 0007 to 0041 and 0060 to 0069; JP 2014-9224 A, paragraphs 0008 to 0048 and 0067 to 0076; JP 2017-119663 A, paragraphs 0013 to 0025; JP 2017-119664 A, paragraphs 0013 to 0026; JP 2017-222623 A, paragraphs 0012 to 0025; JP 2017-226838 A, paragraphs 0010 to 0050; JP 2018-100411 A, paragraphs 0012 to 0043; WO2018/047853, paragraphs 0016 to 0044; and exemplary compounds therein capable of emitting delayed fluorescence are especially preferred. In addition, light-emitting materials capable of emitting delayed fluorescence, as described in JP 2013-253121 A, WO2013/133359, WO2014/034535, WO2014/115743, WO2014/122895, WO2014/126200, WO2014/136758, WO2014/133121, WO2014/136860, WO2014/196585, WO2014/189122, WO2014/168101, WO2015/008580, WO2014/203840, WO2015/002213, WO2015/016200, WO2015/019725, WO2015/072470, WO2015/108049, WO2015/080182, WO2015/072537, WO2015/080183, JP 2015-129240 A, WO2015/129714, WO2015/129715, WO2015/133501, WO2015/136880, WO2015/137244, WO2015/137202, WO2015/137136, WO2015/146541 and WO2015/159541, are also preferably employed. These patent publications described in this paragraph are hereby incorporated as a part of this description by reference.

In some embodiments, the light emitting layer contains the compound represented by the general formula (1) and a light emitting material having a structure other than the general formula (1), and preferably further contains a host material. Light emission from the organic light emitting device of this embodiment contains at least light emission derived from the light emitting material other than the compound represented by the general formula (1). Light emission from the organic light emitting device may contain light emission from the compound represented by the general formula (1) and a host material, in addition to light emission from the light emitting material, but preferably, the light emission amount from the light emitting material is the maximum among the light emission from the organic light emitting device. Also preferably, the light emitting layer in this embodiment is so constituted that the compound represented by the general formula (1) functions as an assist dopant.

Here, the “assist dopant” is one that exhibits a function of moving its own excited energy to a light emitting material to assist light emission of the light emitting material. The excited energy moved to the light emitting material from the assist dopant preferably contains at least an excited singlet energy. The excited singlet energy contains at least any of an excited singlet energy directly formed by the assist dopant through photoexcitation or current-excitation, an excited singlet energy formed by reverse intersystem crossing from an excited triplet state to an excited singlet state, and a host derived excited singlet energy transferred to the assist dopant from a host material. The energy in an excited triplet state that undergoes reverse intersystem crossing in the assist dopant may be an excited triplet energy directly formed in the assist dopant by photoexcitation or current-excitation, or may also be a host-derived excited triplet energy transferred to the assist dopant from a host material. The compound represented by the general formula (1) may readily undergo reverse intersystem crossing from an excited triplet state to an excited singlet state to efficiency produce an excited singlet energy, and therefore may effectively assist the light emission of a light emitting material. In the case where the compound represented by the general formula (1) is used as an assist dopant, the light emitting material to be combined with the assist dopant is preferably a fluorescence emitting material having a lower lowest excited singlet energy level than that of the compound to be used as the assist dopant, and is more preferably a fluorescence emitting material whose lowest excited singlet energy level and lowest excited triplet energy level are both lower than those of the compound to be used as the assist dopant. With that, supply of an excited singlet energy to the light emitting material from the compound of the present invention may be attained efficiently. In addition, the wavelength of the light emitting material can be selected in accordance with the intended use. For example, in the case of use for imaging and sensing targeted for a living body, the light emitting material is preferably a fluorescence emitting material having an emission peak in a wavelength range with high biopermeability (680 to 1800 nm, preferably 680 to 1350 nm, more preferably 680 to 930 nm). Specific examples of the fluorescence emitting material include BBTDTPA used in Examples given hereinunder. In the case where the compound represented by the general formula (1) is used as an assist dopant, the host material to be combined with the assist dopant is preferably a compound having a higher lowest excited singlet energy level than that of the compound as the assist dopant, and is preferably a compound whose lowest excited singlet energy level and lowest excited triplet energy level are both higher than those of the compound as the assist dopant.

The content of the assist dopant in the light emitting layer is preferably smaller than the content of the host material and larger than the content of the light emitting material, that is, the content preferably satisfies a relation of “content of the light emitting material<content of the assist dopant<content of the host material”. Specifically, the content of the assist dopant in the light emitting layer in this embodiment is preferably less than 50% by weight. Further, the upper limit of the content of the assist dopant is preferably less than 40% by mass, and the upper limit of the content may be, for example, less than 30% by weight, less than 20% by weight, or less than 10% by weight. The lower limit is preferably 0.1% by weight or more, and may be, for example, more than 1% by weight, or more than 3% by weight.

In the following, the constituent members and the other layers than the light-emitting layer of the organic electroluminescent device are described.

### Substrate:

In some embodiments, the organic electroluminescent device of the invention is supported by a substrate, wherein the substrate is not particularly limited and may be any of those that have been commonly used in an organic electroluminescent device, for example those formed of glass, transparent plastics, quartz and silicon.

### Anode

In some embodiments, the anode of the organic electroluminescent device is made of a metal, an alloy, an electroconductive compound, or a combination thereof. In some embodiments, the metal, alloy, or electroconductive compound has a large work function (4 eV or more). In some embodiments, the metal is Au. In some embodiments, the electroconductive transparent material is selected from CuI, indium tin oxide (ITO), SnO₂, and ZnO. In some embodiments, an amorphous material capable of forming a transparent electroconductive film, such as IDIXO (In₂O₃—ZnO), is be used. In some embodiments, the anode is a thin film. In some embodiments the thin film is made by vapor deposition or sputtering. In some embodiments, the film is patterned by a photolithography method. In some embodiments, where the pattern may not require high accuracy (for example, approximately 100 μm or more), the pattern may be formed with a mask having a desired shape on vapor deposition or sputtering of the electrode material. In some embodiments, when a material can be applied as a coating, such as an organic electroconductive compound, a wet film forming method, such as a printing method and a coating method is used. In some embodiments, when the emitted light goes through the anode, the anode has a transmittance of more than 10%, and the anode has a sheet resistance of several hundred Ohm per square or less. In some embodiments, the thickness of the anode is from 10 to 1,000 nm. In some embodiments, the thickness of the anode is from 10 to 200 nm. In some embodiments, the thickness of the anode varies depending on the material used.

### Cathode

In some embodiments, the cathode is made of an electrode material a metal having a small work function (4 eV or less) (referred to as an electron injection metal), an alloy, an electroconductive compound, or a combination thereof. In some embodiments, the electrode material is selected from sodium, a sodium-potassium alloy, magnesium, lithium, a magnesium-cupper mixture, a magnesium-silver mixture, a magnesium-aluminum mixture, a magnesium-indium mixture, an aluminum-aluminum oxide (Al₂O₃) mixture, indium, a lithium-aluminum mixture, and a rare earth metal. In some embodiments, a mixture of an electron injection metal and a second metal that is a stable metal having a larger work function than the electron injection metal is used. In some embodiments, the mixture is selected from a magnesium-silver mixture, a magnesium-aluminum mixture, a magnesium-indium mixture, an aluminum-aluminum oxide (Al₂O₃) mixture, a lithium-aluminum mixture, and aluminum. In some embodiments, the mixture increases the electron injection property and the durability against oxidation. In some embodiments, the cathode is produced by forming the electrode material into a thin film by vapor deposition or sputtering. In some embodiments, the cathode has a sheet resistance of several hundred Ohm per square or less. In some embodiments, the thickness of the cathode ranges from 10 nm to 5 μm. In some embodiments, the thickness of the cathode ranges from 50 to 200 nm. In some embodiments, for transmitting the emitted light, any one of the anode and the cathode of the organic electroluminescent device is transparent or translucent. In some embodiments, the transparent or translucent electroluminescent devices enhances the light emission luminance.

In some embodiments, the cathode is formed with an electroconductive transparent material, as described for the anode, to form a transparent or translucent cathode. In some embodiments, a device comprises an anode and a cathode, both being transparent or translucent.

### Injection Layer

An injection layer is a layer between the electrode and the organic layer. In some embodiments, the injection layer decreases the driving voltage and enhances the light emission luminance. In some embodiments the injection layer includes a hole injection layer and an electron injection layer. The injection layer can be positioned between the anode and the light-emitting layer or the hole transporting layer, and between the cathode and the light-emitting layer or the electron transporting layer. In some embodiments, an injection layer is present. In some embodiments, no injection layer is present.

Preferred compound examples for use as a hole injection material are shown below.

### MoO₃,

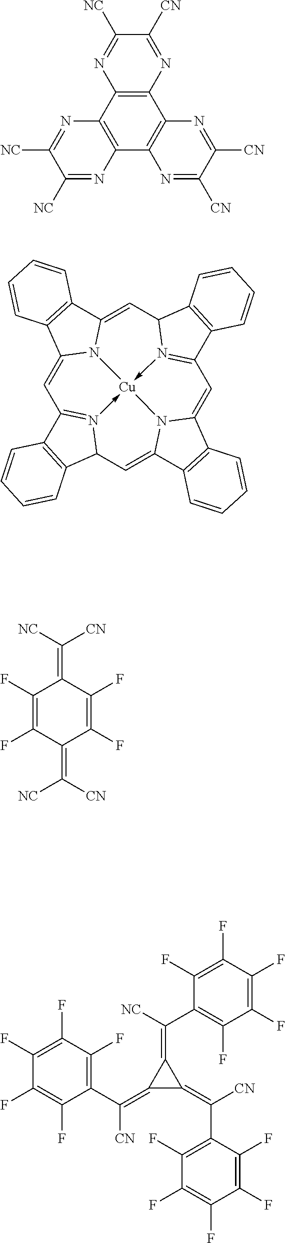

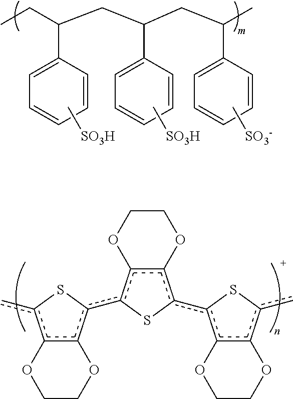

Next, preferred compound examples for use as an electron injection material are shown below.

LiF, CsF,

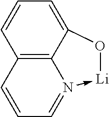

### Barrier Layer

A barrier layer is a layer capable of inhibiting charges (electrons or holes) and/or excitons present in the light-emitting layer from being diffused outside the light-emitting layer. In some embodiments, the electron barrier layer is between the light-emitting layer and the hole transporting layer, and inhibits electrons from passing through the light-emitting layer toward the hole transporting layer. In some embodiments, the hole barrier layer is between the light-emitting layer and the electron transporting layer, and inhibits holes from passing through the light-emitting layer toward the electron transporting layer. In some embodiments, the barrier layer inhibits excitons from being diffused outside the light-emitting layer. In some embodiments, the electron barrier layer and the hole barrier layer are exciton barrier layers. As used herein, the term “electron barrier layer” or “exciton barrier layer” includes a layer that has the functions of both electron barrier layer and of an exciton barrier layer.

### Hole Barrier Layer

A hole barrier layer acts as an electron transporting layer. In some embodiments, the hole barrier layer inhibits holes from reaching the electron transporting layer while transporting electrons. In some embodiments, the hole barrier layer enhances the recombination probability of electrons and holes in the light-emitting layer. The material for the hole barrier layer may be the same materials as the ones described for the electron transporting layer.

Preferred compound examples for use for the hole barrier layer are shown below.

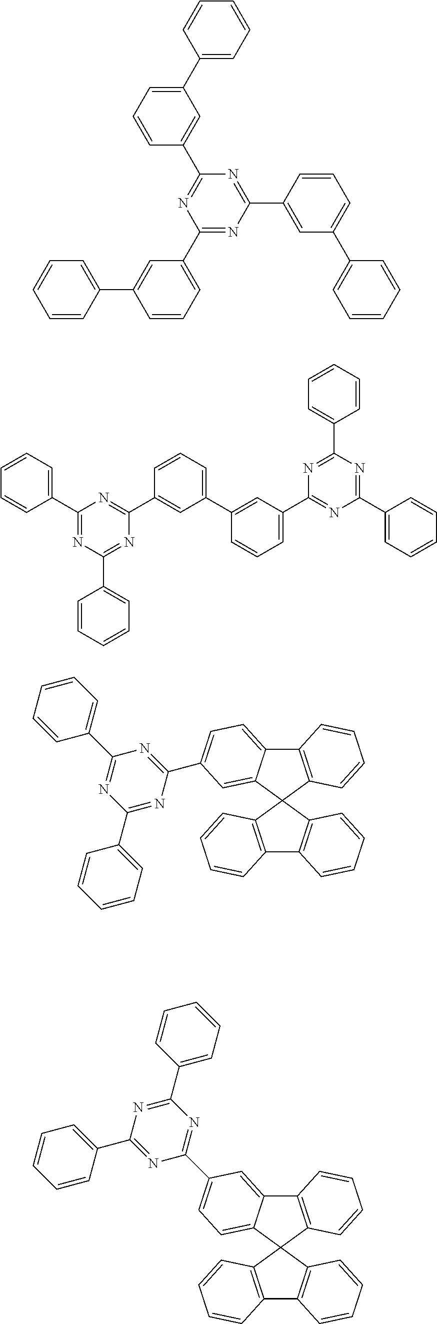

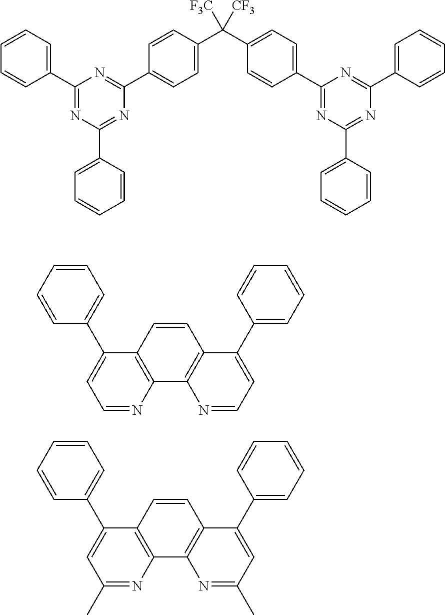

### Electron Barrier Layer

As electron barrier layer transports holes. In some embodiments, the electron barrier layer inhibits electrons from reaching the hole transporting layer while transporting holes. In some embodiments, the electron barrier layer enhances the recombination probability of electrons and holes in the light-emitting layer.

Preferred compound examples for use as the electron barrier material are shown below.

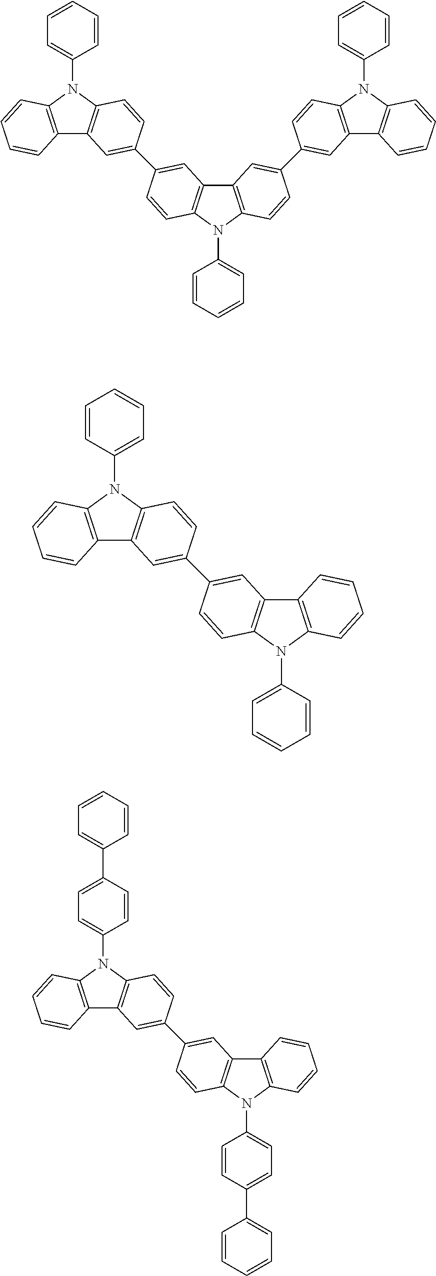 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

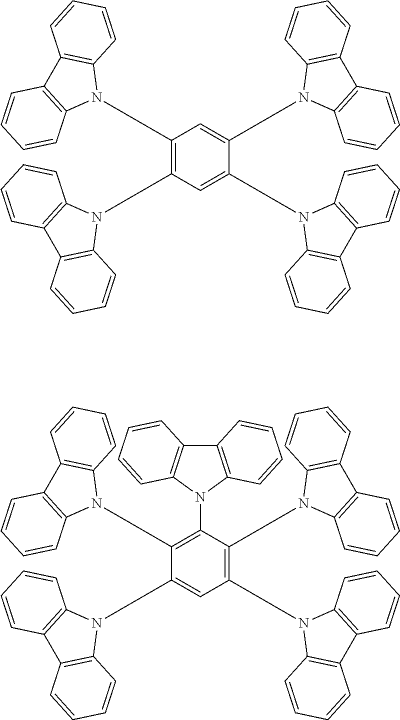 [SMILES: c1ccc2c(c1)c1ccccc1n2-c1cc(-n2c3ccccc3c3ccccc32)c(-n2c3ccccc3c3ccccc32)cc1-n1c2ccccc2c2ccccc21 ; c1ccc2c(c1)c1ccccc1n2-c1cc(-n2c3ccccc3c3ccccc32)c(-n2c3ccccc3c3ccccc32)c(-n2c3ccccc3c3ccccc32)c1-n1c2ccccc2c2ccccc21]

### Exciton Barrier Layer

An exciton barrier layer inhibits excitons generated through recombination of holes and electrons in the light-emitting layer from being diffused to the charge transporting layer. In some embodiments, the exciton barrier layer enables effective confinement of excitons in the light-emitting layer. In some embodiments, the light emission efficiency of the device is enhanced. In some embodiments, the exciton barrier layer is adjacent to the light-emitting layer on any of the side of the anode and the side of the cathode, and on both the sides. In some embodiments, where the exciton barrier layer is on the side of the anode, the layer can be between the hole transporting layer and the light-emitting layer and adjacent to the light-emitting layer. In some embodiments, where the exciton barrier layer is on the side of the cathode, the layer can be between the light-emitting layer and the cathode and adjacent to the light-emitting layer. In some embodiments, a hole injection layer, an electron barrier layer, or a similar layer is between the anode and the exciton barrier layer that is adjacent to the light-emitting layer on the side of the anode. In some embodiments, a hole injection layer, an electron barrier layer, a hole barrier layer, or a similar layer is between the cathode and the exciton barrier layer that is adjacent to the light-emitting layer on the side of the cathode. In some embodiments, the exciton barrier layer comprises excited singlet energy and excited triplet energy, at least one of which is higher than the excited singlet energy and the excited triplet energy of the light-emitting material, respectively.

### Hole Transporting Layer

The hole transporting layer comprises a hole transporting material. In some embodiments, the hole transporting layer is a single layer. In some embodiments, the hole transporting layer comprises a plurality layers.

In some embodiments, the hole transporting material has one of injection or transporting property of holes and barrier property of electrons. In some embodiments, the hole transporting material is an organic material. In some embodiments, the hole transporting material is an inorganic material. Examples of known hole transporting materials that may be used herein include but are not limited to a triazole derivative, an oxadiazole derivative, an imidazole derivative, a carbazole derivative, an indolocarbazole derivative, a polyarylalkane derivative, a pyrazoline derivative, a pyrazolone derivative, a phenylenediamine derivative, an arylamine derivative, an amino-substituted chalcone derivative, an oxazole derivative, a styrylanthracene derivative, a fluorenone derivative, a hydrazone derivative, a stilbene derivative, a silazane derivative, an aniline copolymer and an electroconductive polymer oligomer, particularly a thiophene oligomer, or a combination thereof. In some embodiments, the hole transporting material is selected from a porphyrin compound, an aromatic tertiary amine compound, and a styrylamine compound. In some embodiments, the hole transporting material is an aromatic tertiary amine compound. Preferred compound examples for use as the hole transporting material are shown below.

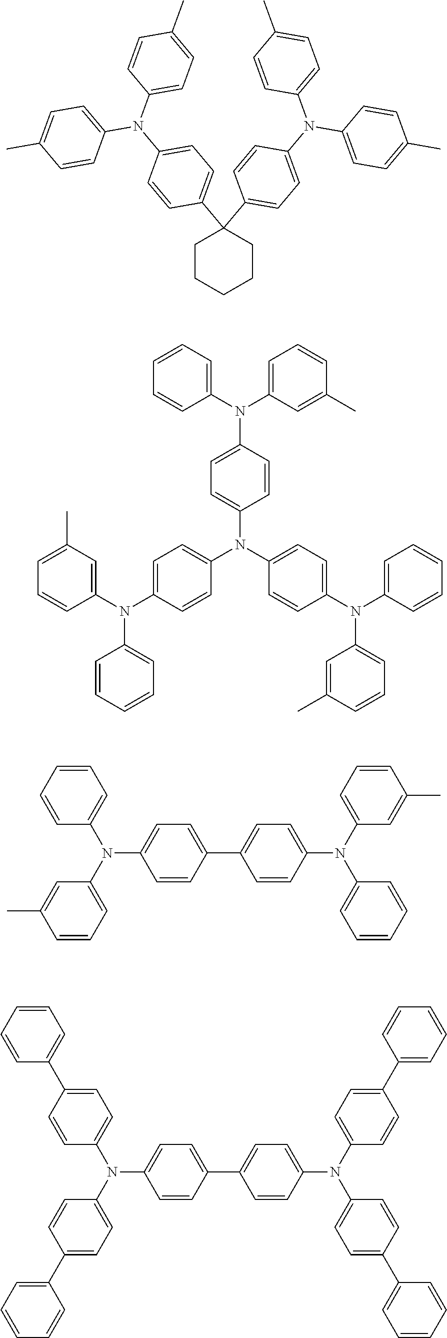

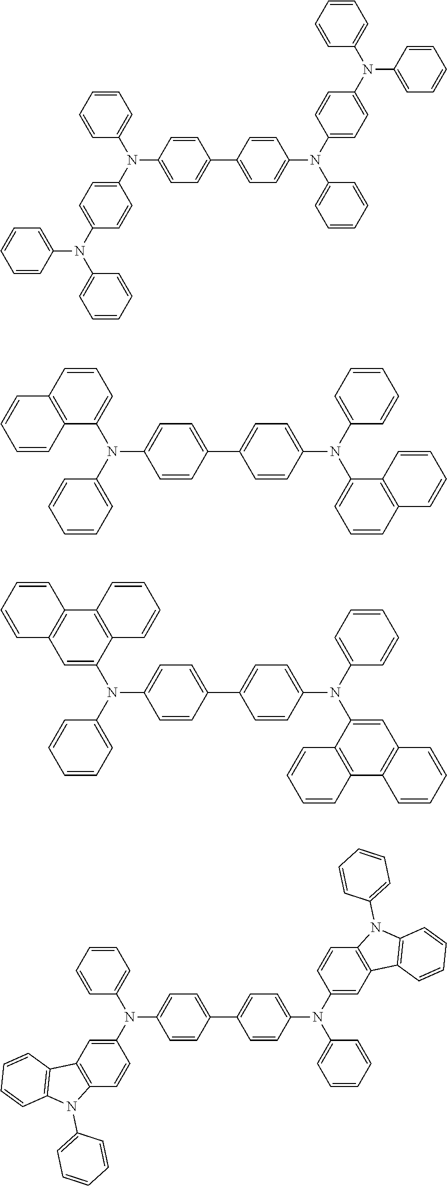 [SMILES: c1ccc(N(c2ccccc2)c2ccc(N(c3ccccc3)c3ccc(-c4ccc(N(c5ccccc5)c5ccc(N(c6ccccc6)c6ccccc6)cc5)cc4)cc3)cc2)cc1 ; c1ccc(N(c2ccc(-c3ccc(N(c4ccccc4)c4cccc5ccccc45)cc3)cc2)c2cccc3ccccc23)cc1 ; c1ccc(N(c2ccc(-c3ccc(N(c4ccccc4)c4cc5ccccc5c5ccccc45)cc3)cc2)c2cc3ccccc3c3ccccc23)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

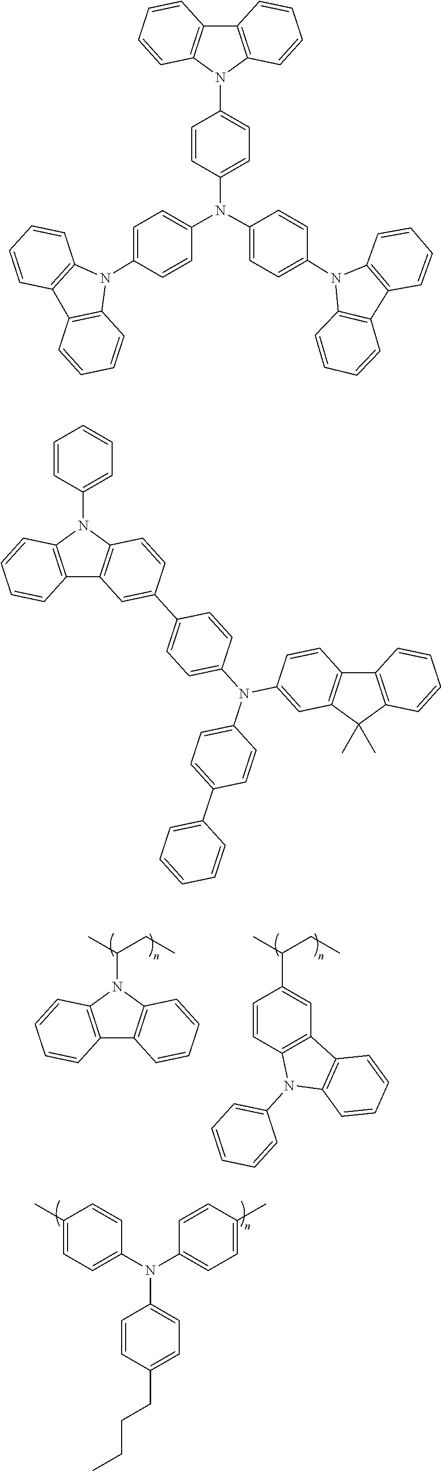

### Electron Transporting Layer

The electron transporting layer comprises an electron transporting material. In some embodiments, the electron transporting layer is a single layer. In some embodiments, the electron transporting layer comprises a plurality of layer.

In some embodiments, the electron transporting material needs only to have a function of transporting electrons, which are injected from the cathode, to the light-emitting layer. In some embodiments, the electron transporting material also function as a hole barrier material. Examples of the electron transporting layer that may be used herein include but are not limited to a nitro-substituted fluorene derivative, a diphenylquinone derivative, a thiopyran dioxide derivative, carbodiimide, a fluorenylidene methane derivative, anthraquinodimethane, an anthrone derivatives, an azole derivative, an azine derivative, an oxadiazole derivative, or a combination thereof, or a polymer thereof. In some embodiments, the electron transporting material is a thiadiazole derivative, or a quinoxaline derivative. In some embodiments, the electron transporting material is a polymer material. Preferred compound examples for use as the electron transporting material are shown below.

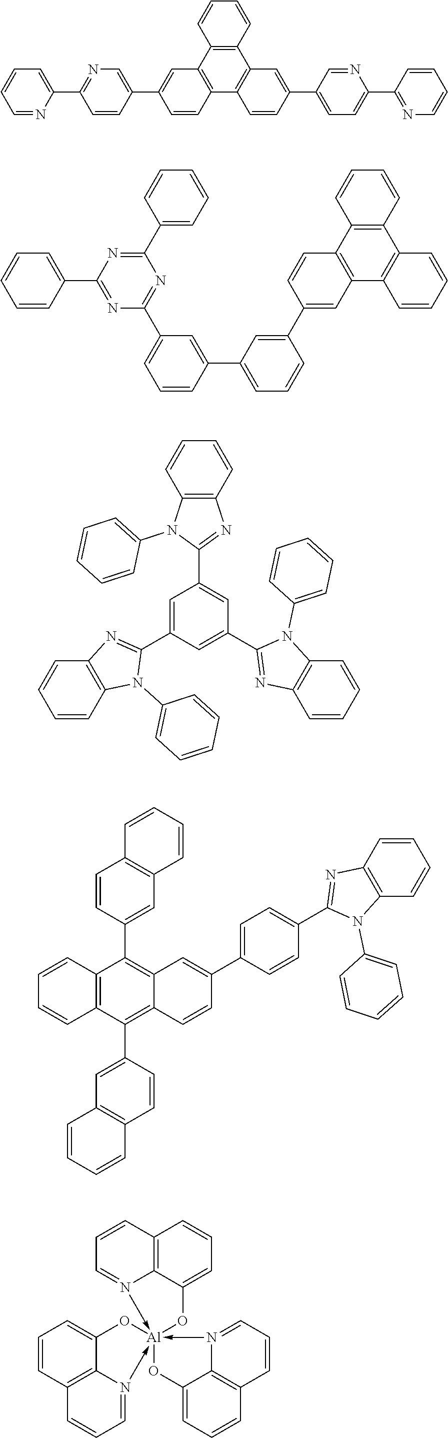

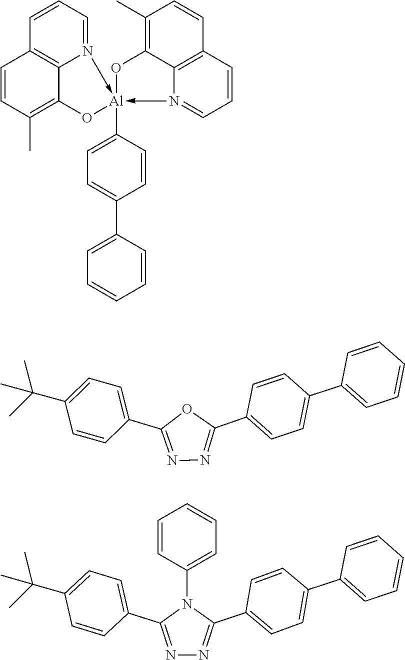

Hereinunder compound examples preferred as a material that can be added to the organic layers are shown. For example, these can be added as a stabilization material.

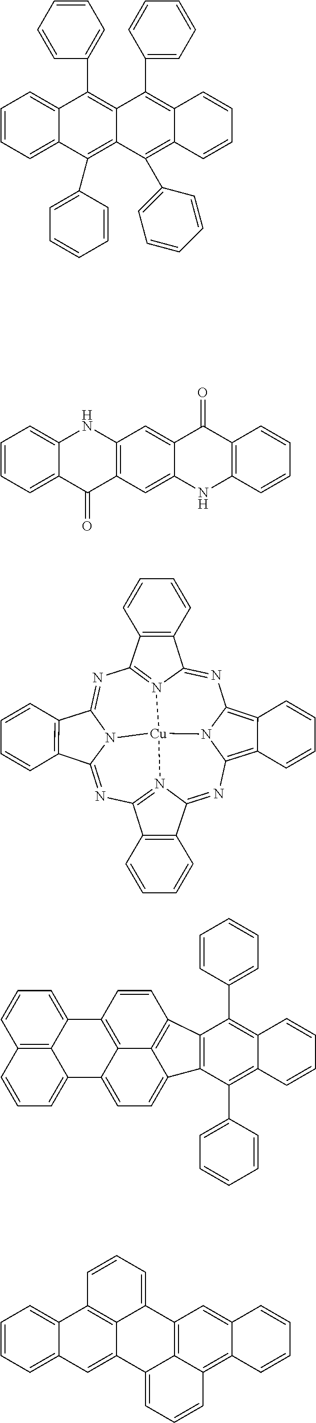

Preferred materials for use in the organic electroluminescent device are specifically shown. However, the materials usable in the invention should not be limitatively interpreted by the following exemplary compounds. Compounds that are exemplified as materials having a specific function can also be used as materials having any other function.

### Devices

In some embodiments, an light emitting layer is incorporated into a device. For example, the device includes, but is not limited to an OLED bulb, an OLED lamp, a television screen, a computer monitor, a mobile phone, and a tablet.

In some embodiments, an electronic device comprises an OLED comprising an anode, a cathode, and at least one organic layer comprising a light emitting layer between the anode and the cathode.

In some embodiments, compositions described herein may be incorporated into various light-sensitive or light-activated devices, such as a OLEDs or photovoltaic devices. In some embodiments, the composition may be useful in facilitating charge transfer or energy transfer within a device and/or as a hole-transport material. The device may be, for example, an organic light-emitting diode (OLED), an organic integrated circuit (O-IC), an organic field-effect transistor (O-FET), an organic thin-film transistor (O-TFT), an organic light-emitting transistor (O-LET), an organic solar cell (O-SC), an organic optical detector, an organic photoreceptor, an organic field-quench device (O-FQD), a light-emitting electrochemical cell (LEC) or an organic laser diode (O-laser).

### Bulbs or Lamps

In some embodiments, an electronic device comprises an OLED comprising an anode, a cathode, and at least one organic layer comprising a light emitting layer between the anode and the cathode.

In some embodiments, a device comprises OLEDs that differ in color. In some embodiments, a device comprises an array comprising a combination of OLEDs. In some embodiments, the combination of OLEDs is a combination of three colors (e.g., RGB). In some embodiments, the combination of OLEDs is a combination of colors that are not red, green, or blue (for example, orange and yellow green). In some embodiments, the combination of OLEDs is a combination of two, four, or more colors.

In some embodiments, a device is an OLED light comprising:
a circuit board having a first side with a mounting surface and an opposing second side, and defining at least one aperture;
at least one OLED on the mounting surface, the at least one OLED configured to emanate light, comprising:
   an anode, a cathode, and at least one organic layer comprising a light emitting layer between the anode and the cathode;
a housing for the circuit board; and
at least one connector arranged at an end of the housing, the housing and the connector defining a package adapted for installation in a light fixture.

In some embodiments, the OLED light comprises a plurality of OLEDs mounted on a circuit board such that light emanates in a plurality of directions. In some embodiments, a portion of the light emanated in a first direction is deflected to emanate in a second direction. In some embodiments, a reflector is used to deflect the light emanated in a first direction.

### Displays or Screens

In some embodiments, the compounds of the invention can be used in a screen or a display. In some embodiments, the compounds of the invention are deposited onto a substrate using a process including, but not limited to, vacuum evaporation, deposition, vapor deposition, or chemical vapor deposition (CVD). In some embodiments, the substrate is a photoplate structure useful in a two-sided etch provides a unique aspect ratio pixel. The screen (which may also be referred to as a mask) is used in a process in the manufacturing of OLED displays. The corresponding artwork pattern design facilitates a very steep and narrow tie-bar between the pixels in the vertical direction and a large, sweeping bevel opening in the horizontal direction. This allows the close patterning of pixels needed for high definition displays while optimizing the chemical deposition onto a TFT backplane.

The internal patterning of the pixel allows the construction of a 3-dimensional pixel opening with varying aspect ratios in the horizontal and vertical directions. Additionally, the use of imaged “stripes” or halftone circles within the pixel area inhibits etching in specific areas until these specific patterns are undercut and fall off the substrate. At that point the entire pixel area is subjected to a similar etch rate but the depths are varying depending on the halftone pattern. Varying the size and spacing of the halftone pattern allows etching to be inhibited at different rates within the pixel allowing for a localized deeper etch needed to create steep vertical bevels.

A preferred material for the deposition mask is invar. Invar is a metal alloy that is cold rolled into long thin sheet in a steel mill. Invar cannot be electrodeposited onto a rotating mandrel as the nickel mask. A preferred and more cost feasible method for forming the open areas in the mask used for deposition is through a wet chemical etching.

In some embodiments, a screen or display pattern is a pixel matrix on a substrate. In some embodiments, a screen or display pattern is fabricated using lithography (e.g., photolithography and e-beam lithography). In some embodiments, a screen or display pattern is fabricated using a wet chemical etch. In further embodiments, a screen or display pattern is fabricated using plasma etching.

### Methods of Manufacturing Devices

An OLED display is generally manufactured by forming a large mother panel and then cutting the mother panel in units of cell panels. In general, each of the cell panels on the mother panel is formed by forming a thin film transistor (TFT) including an active layer and a source/drain electrode on a base substrate, applying a planarization film to the TFT, and sequentially forming a pixel electrode, a light-emitting layer, a counter electrode, and an encapsulation layer, and then is cut from the mother panel.

An OLED display is generally manufactured by forming a large mother panel and then cutting the mother panel in units of cell panels. In general, each of the cell panels on the mother panel is formed by forming a thin film transistor (TFT) including an active layer and a source/drain electrode on a base substrate, applying a planarization film to the TFT, and sequentially forming a pixel electrode, a light-emitting layer, a counter electrode, and an encapsulation layer, and then is cut from the mother panel.

In another aspect, provided herein is a method of manufacturing an organic light-emitting diode (OLED) display, the method comprising:
forming a barrier layer on a base substrate of a mother panel;
forming a plurality of display units in units of cell panels on the barrier layer;
forming an encapsulation layer on each of the display units of the cell panels;
applying an organic film to an interface portion between the cell panels.

In some embodiments, the barrier layer is an inorganic film formed of, for example, SiNx, and an edge portion of the barrier layer is covered with an organic film formed of polyimide or acryl. In some embodiments, the organic film helps the mother panel to be softly cut in units of the cell panel.

In some embodiments, the thin film transistor (TFT) layer includes a light-emitting layer, a gate electrode, and a source/drain electrode. Each of the plurality of display units may include a thin film transistor (TFT) layer, a planarization film formed on the TFT layer, and a light-emitting unit formed on the planarization film, wherein the organic film applied to the interface portion is formed of a same material as a material of the planarization film and is formed at a same time as the planarization film is formed. In some embodiments, a light-emitting unit is connected to the TFT layer with a passivation layer and a planarization film therebetween and an encapsulation layer that covers and protects the light-emitting unit. In some embodiments of the method of manufacturing, the organic film contacts neither the display units nor the encapsulation layer.

Each of the organic film and the planarization film may include any one of polyimide and acryl. In some embodiments, the barrier layer may be an inorganic film. In some embodiments, the base substrate may be formed of polyimide. The method may further include, before the forming of the barrier layer on one surface of the base substrate formed of polyimide, attaching a carrier substrate formed of a glass material to another surface of the base substrate, and before the cutting along the interface portion, separating the carrier substrate from the base substrate. In some embodiments, the OLED display is a flexible display.

In some embodiments, the passivation layer is an organic film disposed on the TFT layer to cover the TFT layer. In some embodiments, the planarization film is an organic film formed on the passivation layer. In some embodiments, the planarization film is formed of polyimide or acryl, like the organic film formed on the edge portion of the barrier layer. In some embodiments, the planarization film and the organic film are simultaneously formed when the OLED display is manufactured. In some embodiments, the organic film may be formed on the edge portion of the barrier layer such that a portion of the organic film directly contacts the base substrate and a remaining portion of the organic film contacts the barrier layer while surrounding the edge portion of the barrier layer.

In some embodiments, the light-emitting layer includes a pixel electrode, a counter electrode, and an organic light-emitting layer disposed between the pixel electrode and the counter electrode. In some embodiments, the pixel electrode is connected to the source/drain electrode of the TFT layer.

In some embodiments, when a voltage is applied to the pixel electrode through the TFT layer, an appropriate voltage is formed between the pixel electrode and the counter electrode, and thus the organic light-emitting layer emits light, thereby forming an image. Hereinafter, an image forming unit including the TFT layer and the light-emitting unit is referred to as a display unit.

In some embodiments, the encapsulation layer that covers the display unit and prevents penetration of external moisture may be formed to have a thin film encapsulation structure in which an organic film and an inorganic film are alternately stacked. In some embodiments, the encapsulation layer has a thin film encapsulation structure in which a plurality of thin films are stacked. In some embodiments, the organic film applied to the interface portion is spaced apart from each of the plurality of display units. In some embodiments, the organic film is formed such that a portion of the organic film directly contacts the base substrate and a remaining portion of the organic film contacts the barrier layer while surrounding an edge portion of the barrier layer.

In one embodiment, the OLED display is flexible and uses the soft base substrate formed of polyimide. In some embodiments, the base substrate is formed on a carrier substrate formed of a glass material, and then the carrier substrate is separated.

In some embodiments, the barrier layer is formed on a surface of the base substrate opposite to the carrier substrate. In one embodiment, the barrier layer is patterned according to a size of each of the cell panels. For example, while the base substrate is formed over the entire surface of a mother panel, the barrier layer is formed according to a size of each of the cell panels, and thus a groove is formed at an interface portion between the barrier layers of the cell panels. Each of the cell panels can be cut along the groove.

In some embodiments, the method of manufacture further comprises cutting along the interface portion, wherein a groove is formed in the barrier layer, wherein at least a portion of the organic film is formed in the groove, and wherein the groove does not penetrate into the base substrate. In some embodiments, the TFT layer of each of the cell panels is formed, and the passivation layer which is an inorganic film and the planarization film which is an organic film are disposed on the TFT layer to cover the TFT layer. At the same time as the planarization film formed of, for example, polyimide or acryl is formed, the groove at the interface portion is covered with the organic film formed of, for example, polyimide or acryl. This is to prevent cracks from occurring by allowing the organic film to absorb an impact generated when each of the cell panels is cut along the groove at the interface portion. That is, if the entire barrier layer is entirely exposed without the organic film, an impact generated when each of the cell panels is cut along the groove at the interface portion is transferred to the barrier layer, thereby increasing the risk of cracks. However, in one embodiment, since the groove at the interface portion between the barrier layers is covered with the organic film and the organic film absorbs an impact that would otherwise be transferred to the barrier layer, each of the cell panels may be softly cut and cracks may be prevented from occurring in the barrier layer. In one embodiment, the organic film covering the groove at the interface portion and the planarization film are spaced apart from each other. For example, if the organic film and the planarization film are connected to each other as one layer, since external moisture may penetrate into the display unit through the planarization film and a portion where the organic film remains, the organic film and the planarization film are spaced apart from each other such that the organic film is spaced apart from the display unit.

In some embodiments, the display unit is formed by forming the light-emitting unit, and the encapsulation layer is disposed on the display unit to cover the display unit. As such, once the mother panel is completely manufactured, the carrier substrate that supports the base substrate is separated from the base substrate. In some embodiments, when a laser beam is emitted toward the carrier substrate, the carrier substrate is separated from the base substrate due to a difference in a thermal expansion coefficient between the carrier substrate and the base substrate.

In some embodiments, the mother panel is cut in units of the cell panels. In some embodiments, the mother panel is cut along an interface portion between the cell panels by using a cutter. In some embodiments, since the groove at the interface portion along which the mother panel is cut is covered with the organic film, the organic film absorbs an impact during the cutting. In some embodiments, cracks may be prevented from occurring in the barrier layer during the cutting.

In some embodiments, the methods reduce a defect rate of a product and stabilize its quality.

Another aspect is an OLED display including: a barrier layer that is formed on a base substrate; a display unit that is formed on the barrier layer; an encapsulation layer that is formed on the display unit; and an organic film that is applied to an edge portion of the barrier layer.

### EXAMPLES

The features of the present invention will be described more specifically with reference to Examples and Comparative Examples given below. The materials, amount used, proportion, processes, procedures and the like shown in the following Examples may be appropriately modified unless they deviate from the substance of the invention. Accordingly, the scope of the invention is not construed as being limited to the specific examples shown below. For photoabsorption spectrometry, used was a spectrophotometer (LAMBDA950-PKA, by Perkin Elmer Co., Ltd.); for evaluation of light emission characteristics, used were a multichannel spectrofluorophotometer (FP-8600, by JASCO Corporation), an absolute PL quantum yield measuring system (C13534-21, by Hamamatsu Photonics K.K.), and a fluorescence lifetime measuring apparatus (C11367, by Hamamatsu Photonics K.K.). For evaluation of EL device characteristics, used were a semiconductor parameter analyzer (E5273A, by Agilent Technology Corporation), a multichannel spectrofluorophotometer (C10027-02, C10028-01, by Hamamatsu Photonics K.K.), a light emission lifetime measuring apparatus (EAS-26B, by System Engineer Corporation), and a source meter (2400 series, by Keithley Corporation).

A difference ΔE_{ST} between the lowest excited singlet energy level E_{S1} and the lowest excited triplet energy level E_{T1} is determined by calculating ΔE_{ST}=E_{S1}−E_{T1} in which E_{S1} and E_{T1} are measured according to the following method.

### (1) Lowest Excited Singlet Energy Level E_{S1}

A compound targeted for measurement and a host material are co-evaporated on an Si substrate to prepare a sample having a thickness of 100 nm thereon in which the concentration of the targeted compound for measurement is 6% by weight. The fluorescent spectrum of the sample is measured at room temperature (300 K). Specifically, emission from immediately after incidence of excitation light to 100 nanoseconds after incidence of excitation light is integrated to obtain a fluorescence spectrum on a graph on which the vertical axis indicates emission intensity and the horizontal axis indicates wavelength. A tangent line is drawn to the rising of the emission spectrum on the short wavelength side, and the wavelength value λedge [nm] at the intersection between the tangent line and the horizontal axis is read. The wavelength value is converted into an energy value according to the following conversion expression to calculate E_{S1}.

Conversion Expression: E_{S1} [eV]=1239.85/λedge

For measurement of emission spectrum, a nitrogen laser (MNL200, by Lasertechnik Berlin Corporation) can be used as an excitation light source, and a streak cameral (C4334, by Hamamatsu Photonics K.K.) can be used as a detector.

### (2) Lowest Excited Triplet Energy Level E_{T1}

The same sample as that for measurement of the lowest excited singlet energy level E_{S1} is cooled to 5 [K], and the sample for phosphorescence measurement is irradiated with excitation light (337 nm), and using a streak camera, the phosphorescence intensity thereof is measured. Specifically, the emission from 1 millisecond after incidence of the excitation light to 10 milliseconds after incidence of the excitation light is integrated to obtain a phosphorescence spectrum on a graph on which the vertical axis indicates emission intensity and the horizontal axis indicates wavelength. A tangent line is drawn to the rising of the phosphorescence spectrum on the short wavelength side, and the wavelength value λedge [nm] at the intersection between the tangent line and the horizontal axis is read. The wavelength value is converted into an energy value according to the following conversion expression to calculate E_{T1}.

Conversion Expression: E_{T1} [eV]=1239.85/λedge

The tangent line to the rising of the phosphorescent spectrum on the short wavelength side is drawn as follows. While moving on the spectral curve from the short wavelength side of the phosphorescent spectrum toward the maximum value on the shortest wavelength side among the maximum values of the spectrum, a tangent line at each point on the curve toward the long wavelength side is taken into consideration. With rising thereof (that is, with increase in the vertical axis), the inclination of the tangent line increases. The tangent line drawn at the point at which the inclination value has a maximum value is referred to as the tangent line to the rising on the short wavelength side of the phosphorescent spectrum.

The maximum point having a peak intensity of 10% or less of the maximum peak intensity of the spectrum is not included in the maximum value on the above-mentioned shortest wavelength side, and the tangent line drawn at the point which is closest to the maximum value on the shortest wavelength side and at which the inclination value has a maximum value is referred to as the tangent line to the rising on the short wavelength side of the phosphorescent spectrum.

### (Synthesis Example 1) Synthesis of Compound 1

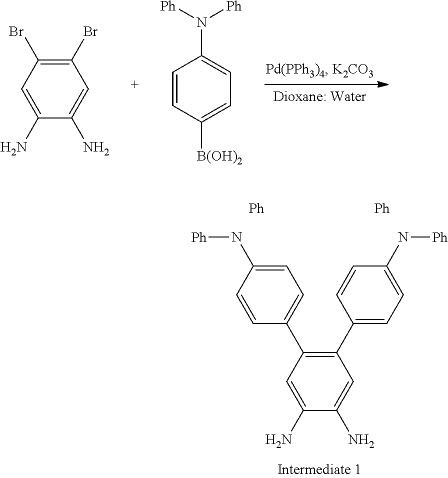 [SMILES: Nc1cc(Br)c(Br)cc1N ; OB(O)c1ccc(N(c2ccccc2)c2ccccc2)cc1 ; Nc1cc(-c2ccc(Nc3ccccc3)cc2)c(-c2ccc(Nc3ccccc3)cc2)cc1N ; "Ph" ; "Ph"]

In a nitrogen atmosphere, 4,5-dibromobenzene-1,2-diamine (1.33 g, 5.0 mmol), (4-(diphenylamino)phenyl)boronic acid (3.47 g, 12 mmol), potassium carbonate (2.76 g, 20 mmol) and tetrakis(triphenylphosphine)palladium(0) (116 mg, 0.1 mmol) were added to a mixed solvent (100 mL) of 1-4-dioxane/water=10/1 (by volume), and stirred at 90° C. for 24 hours. The mixture was cooled down to room temperature, poured into water (100 mL), and extracted with dichloromethane. The resultant organic layer was distilled under reduced pressure, and the resultant residue was purified by column chromatography using a mixed solvent of n-hexane and ethyl acetate=2/1 (by volume) as an eluent. By the above process, a white solid of Intermediate 1 (N⁴,N⁴,N^{4″},N^{4″}-tetraphenyl-[1,1′:2′,1″-terphenyl]-4,4′,4″,5′-tetramine) was obtained at a yield of 2.32 g, 78%.

¹H NMR (500 MHz, DMSO-d₆) δ 4.61 (s, 4H, —NH₂) 7.21 (s, 2H, Ar H), 6.83 (d, J=10.0 Hz, 4H, Ar H), 6.95 (m, 12H, Ar H), 6.99 (t, J=10.0 Hz, 4H, Ar H), 7.23 (t, J=10.0 Hz, 8H, Ar H).

¹³C NMR (125 MHz, CDCl₃, δ): 115.35, 121.93, 122.65, 122.85, 127.98, 128.78, 129.95, 133.85, 136.77, 143.88, 146.69.

ASAP-MS: m/z: calculated for C₄₂H₃₄N₄: 594.76. found: 594.56.

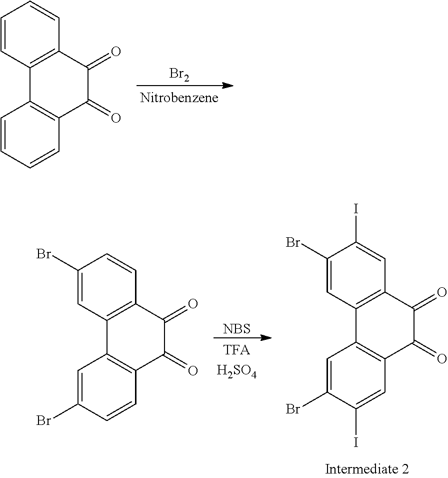

Phenanthrenequinone, bromine and nitrobenzene were mixed and reacted to synthesize 3,6-dibromophenanthrenequinone. Next, 3,6-dibromophenanthrenequinone, N-iodosuccinimide, trifluoroacetic acid and sulfuric acid were mixed and reacted to synthesize Intermediate 2. Here, the reaction was carried out in accordance with the method described in J. Am. Chem. Soc. 2006, 128, 4854.

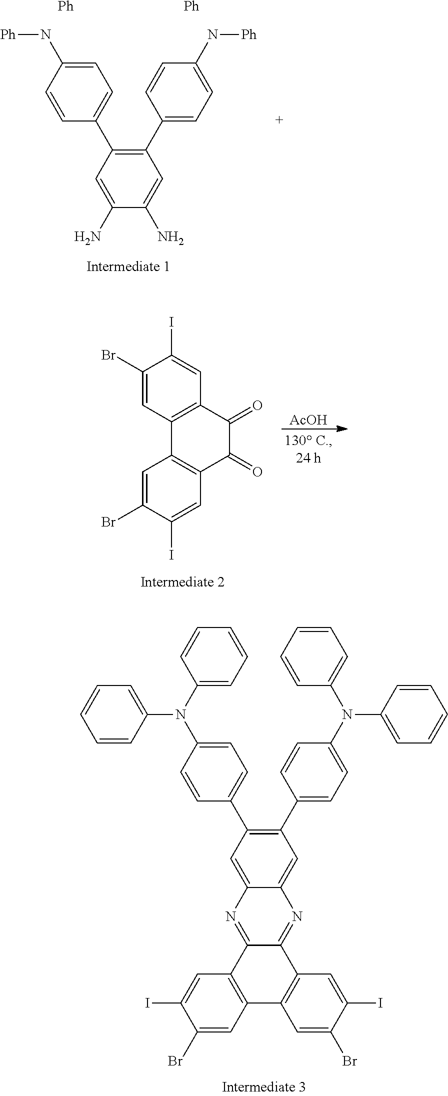

In a nitrogen atmosphere, Intermediate 1 (1.15 g, 1.94 mmol) and Intermediate 2 (1.0 g, 1.62 mmol) were added to acetic acid (250 mL), and stirred at 125° C. for 24 hours. The mixture was cooled down to room temperature, and mixed with water with ice. The precipitated solid was collected by filtration under reduced pressure, washed with methanol and recrystallized with chloroform to obtain a dark red solid of Intermediate 3 (4,4′-(3,6-dibromo-2,7-diiododibenzo[a,c]phenazine-11,12-diyl)-bis(N,N-diphenylaniline)) at a yield of 1.71 g, 90%.

¹H NMR (400 MHz, CD₂Cl₂) δ 7.02 (t, J=5.0 Hz, 4H, Ar H), 7.07 (d, J=5.0 Hz, 12H, Ar H), 7.23 (t, J=5.0 Hz, 8H, Ar H), 7.54 (d, J=10.0 Hz, 4H, Ar H), 7.60 (d, J=5.0 Hz, 4H, Ar H), 8.11 (t, J=5.0 Hz, 2H, Ar H), 8.21 (s, 2H, Ar H).

MALDI-TOF-MS: m/z: calculated for C₅₆H₃₄Br₂I₂N₄: 1176.53. found: 1177.05.

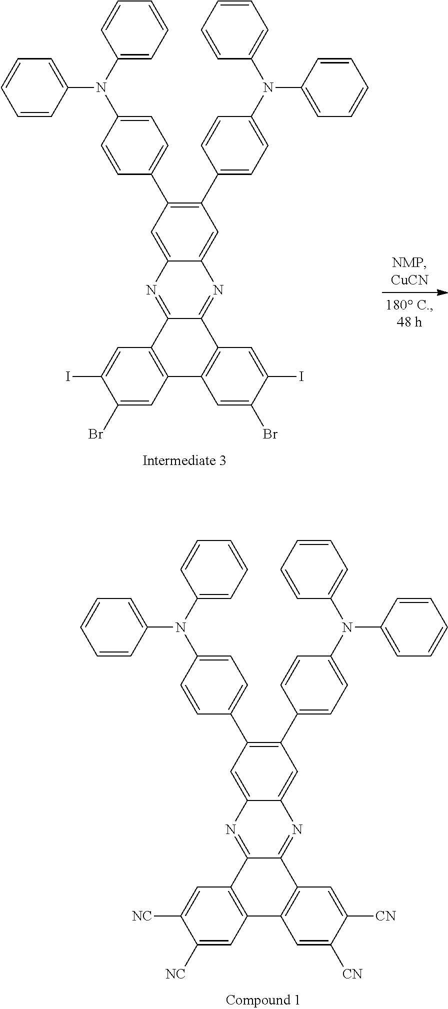 [SMILES: Brc1cc2c3cc(Br)c(I)cc3c3nc4cc(-c5ccc(N(c6ccccc6)c6ccccc6)cc5)c(-c5ccc(N(c6ccccc6)c6ccccc6)cc5)cc4nc3c2cc1I ; N#Cc1cc2c3cc(C#N)c(C#N)cc3c3nc4cc(-c5ccc(N(c6ccccc6)c6ccccc6)cc5)c(-c5ccc(N(c6ccccc6)c6ccccc6)cc5)cc4nc3c2cc1C#N]

In an argon atmosphere, Intermediate 3 (1.5 g, 1.25 mmol) and copper(I) cyanide (CuCN: 1.12 g, 12.5 mmol) were added to anhydrous N-methyl-2-pyrrolidone (150 mL), and stirred at 180° C. for 48 hours. The mixture was cooled down to room temperature, then added with an aqueous solution of 2% ammonia (500 mL), and extracted three times with toluene (50 mL). The resultant organic layer was washed with water, and dried with magnesium sulfate. The crude product was purified by column chromatography using a mixed solvent of toluene/chloroform=9/1 (by volume) as an eluent to obtain a dark blue solid of the intended product, Compound 1 (11,12-bis(4-(diphenylamino)phenyl)dibenzo[a,c]phenazine-2,3,6,7-tetracarbonitrile) at a yield of 820 mg, 76%.

¹H NMR (400 MHz, CDCl₃, δ): 7.08 (m, 8H, Ar H), 7.16 (d, J=5.0 Hz, 8H; Ar H), 7.21 (d, J=5.0 Hz, 4H; Ar H), 7.29 (m, 8H, Ar H), 8.44 (s, 2H, Ar H), 9.00 (s, 2H, Ar H), 9.95 (s, 2H, Ar H);

¹³C NMR (125 MHz, CDCl₃, δ): 115.02, 115.90, 116.03, 122.12, 123.61, 125.01, 129.47, 129.67, 130.72, 131.29, 132.55, 132.75, 134.82, 139.23, 143.04, 147.17, 147.30, 148.01;

MALDI-TOF-MS: m/z: calculated for C₆₀H₃₄N₈: 866.9880. found: 867.25.

Anal. calcd for C₆₀H₃₄N₈: C, 83.12; H, 3.95; N, 12.92. found: C, 83.04; H, 3.95; N, 12.84.

### (Synthesis Example 2) Synthesis of Compound 2

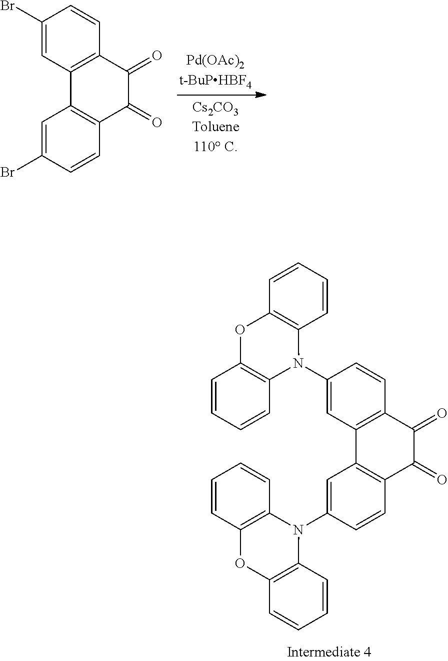 [SMILES: O=C1C(=O)c2ccc(Br)cc2-c2cc(Br)ccc21 ; O=C1C(=O)c2ccc(N3c4ccccc4Oc4ccccc43)cc2-c2cc(N3c4ccccc4Oc4ccccc43)ccc21]

3,6-Dibromophenanthrenequinone (3.66 g, 10.0 mmol) obtained in the same manner as Synthesis Example 1, phenoxazine (4.03 g, 22 mmol), tri-tert-butylphosphonium tetrafluoroborate (435 mg, 1.5 mmol), cesium carbonate (13.0 g, 40 mmol) and palladium(II) acetate (112 mg, 0.5 mmol) were added to toluene (200 mL) in a nitrogen atmosphere, and stirred at 110° C. for 24 hours. The mixture was cooled down to room temperature, then poured into water (100 mL), and extracted with dichloromethane. The solvent was evaporated away under reduced pressure from the resultant organic layer, and the resultant crude product was purified by column chromatography using a mixed solvent of n-hexane/chloroform=1/1 (by volume) as an eluent. By the above process, a black solid of Intermediate 4 (3,6-di(10H-phenoxazin-10-yl)phenanthrene-9,10-dione) at a yield of 4.65 g, 81%.

¹H NMR (500 MHz, CDCl₃) δ 6.11 (d, J=10.0 Hz, 4H, Ar H), 6.65 (t, J=7.5 Hz, 4H, Ar H), 6.75 (in, 8H, Ar H), 7.54 (d, J=5.0 Hz, 2H, Ar H), 7.95 (s, 2H, Ar H), 8.46 (d, J=10.0 Hz, 2H, Ar H).

¹³C NMR (125 MHz, CDCl₃, δ): 178.88, 113.80, 116.19, 122.68, 123.46, 126.02, 130.19, 131.99, 132.92, 133.61, 138.06, 144.43, 147.22;

MALDI-TOF-MS: m/z: calculated for C₃₈H₂₂N₂O₄: 570.6040, found: 572.45.

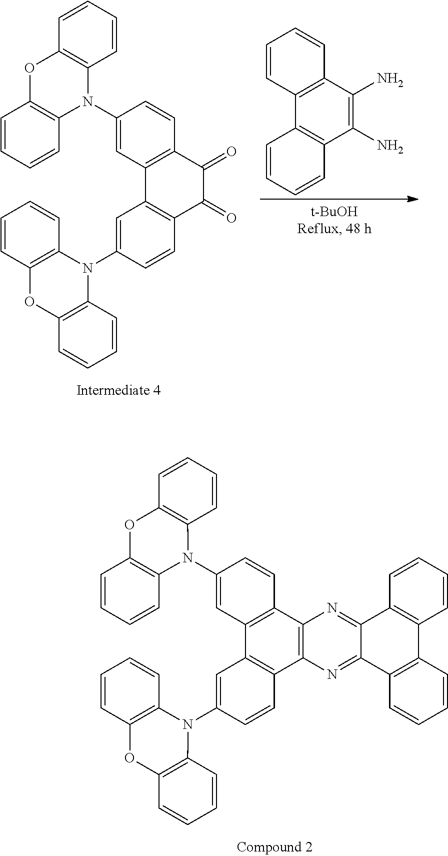

In a nitrogen atmosphere, Intermediate 4 (579 mg, 1.0 mmol) and phenanthrene-9,10-diamine (250 mg, 1.2 mmol) were added to tert-butyl alcohol (100 mL), and stirred at 105° C. for 48 hours. The mixture was cooled down to room temperature, and mixed with water with ice. The precipitated solid was collected by filtration under reduced pressure, washed with methanol and purified by column chromatography. The purified fraction was recrystallized with chloroform to obtain a deep yellow solid of the intended product, Compound 2 at a yield of 550 mg, 74%.

¹H NMR (500 MHz, CDCl₃) δ 6.07 (d, J=10.0 Hz, 4H, Ar H), 6.59 (t, J=7.5 Hz, 4H, Ar H), 6.67 (t, J=10.0 Hz, 4H, Ar H), 6.74 (d, J=10.0 Hz, 4H, Ar H), 7.87 (m, 6H, Ar H), 8.58 (s, 2H, Ar H), 7.71 (d, J=10.0 Hz, 2H, Ar H), 9.64 (d, J=10.0 Hz, 2H, Ar H), 9.88 (d, J=10.0 Hz, 2H, Ar H).

¹³C NMR (125 MHz, CDCl₃, δ): 113.37, 115.68, 121.70, 123.09, 123.35, 125.80, 126.08, 128.01, 129.30, 130.22, 130.52, 130.84, 132.08, 133.63, 134.25, 140.63, 141.21, 144.04.

MALDI-TOF-MS: m/z: calculated for C₅₂H₃₀N₄O₂: 742.8380, found: 743.10.

### (Synthesis Example 3) Synthesis of Compound 3

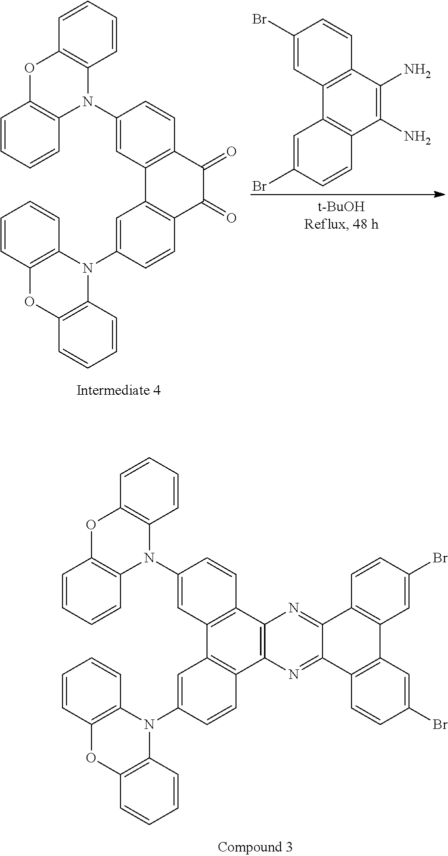

In a nitrogen atmosphere, Intermediate 4 (1.14 mg, 2.0 mmol) obtained in the same manner as in Synthesis Example 2 and 3,6-dibromophenanthrene-9,10-diamine (1.10 g, 3.0 mmol) were added to tert-butyl alcohol (200 mL), and stirred at 105° C. for 48 hours. The mixture was cooled down to room temperature, and mixed with water with ice. The precipitated solid was collected by filtration under reduced pressure, washed with methanol, and purified by column chromatography. The purified fraction was recrystallized with chloroform to obtain a red solid of the intended product, Compound 3 at a yield of 1.60 g, 83%.

¹H NMR (500 MHz, CDCl₃) δ 6.08 (d, J=10.0 Hz, 4H, Ar H), 6.61 (t, J=7.5 Hz, 4H, Ar H), 6.70 (t, J=10.0 Hz, 4H, Ar H), 6.77 (d, J=10.0 Hz, 4H, Ar H), 7.88 (d, J=10.0 Hz, 2H, Ar H), 8.02 (d, J=10.0 Hz, 2H, Ar H), 8.60 (s, 2H, Ar H), 8.76 (s, 2H, Ar H), 9.50 (d, J=10.0 Hz, 2H, Ar H), 9.84 (d, J=10.0 Hz, 2H, Ar H).

MALDI-TOF-MS: m/z: calculated for C₅₂H₂₈Br₂N₄O₂: 900.6300, found: 901.48.

### (Synthesis Example 4) Synthesis of Compound 4

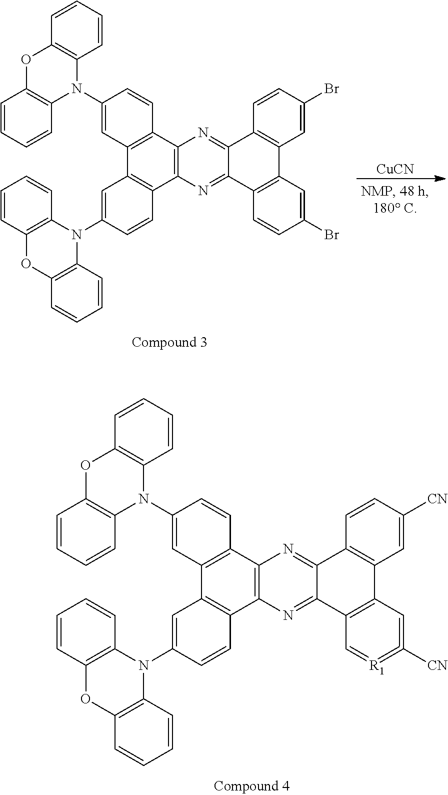

In an argon atmosphere, Compound 3 (900 mg, 1.0 mmol) obtained in the same manner as in Synthesis Example 3 and copper(I) cyanide (890 mg, 10.0 mmol) were added to anhydrous N-methyl-2-pyrrolidone (150 mL), and heated at 180° C. for 48 hours. The mixture was cooled down to room temperature, added with aqueous 2% ammonia (500 mL), and extracted three times with toluene (50 ml). The resultant organic layer was washed with water, and dried with magnesium sulfate. The crude product was purified by column chromatography using a mixed solvent of toluene/chloroform=9/1 (by volume) as an eluent to obtain a dark brown solid of the intended product, Compound 4 at a yield of 620 mg, 78%.

¹H NMR (500 MHz, CDCl₃) δ 6.01 (d, J=10.0 Hz, 4H, Ar H), 6.51 (t, J=7.5 Hz, 4H, Ar H), 6.60 (t, J=10.0 Hz, 4H, Ar H), 6.65 (d, J=10.0 Hz, 4H, Ar H), 7.83 (d, J=10.0 Hz, 2H, Ar H), 8.08 (d, J=10.0 Hz, 2H, Ar H), 8.55 (s, 2H, Ar H), 8.92 (s, 2H, Ar H), 9.68 (d, J=10.0 Hz, 2H, Ar H), 9.77 (d, J=10.0 Hz, 2H, Ar H); ¹³C NMR (125 MHz, CDCl₃, δ): 113.29, 115.84, 121.95, 123.34, 126.11, 129.62, 130.15, 130.57, 131.05, 131.37, 133.51, 134.02, 144.06;

MALDITOF-MS: m/z: calculated for C₅₄H₂₈N₆O₂: 792.8580, found: 796.16.

### (Synthesis Example 5) Synthesis of Compound 5

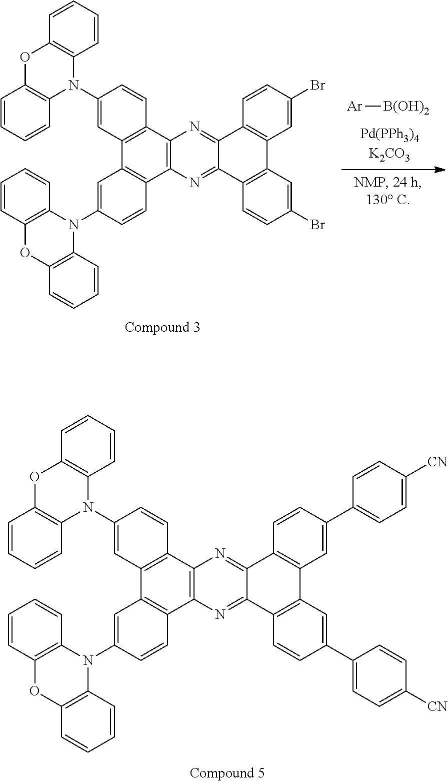

In a nitrogen atmosphere, Compound 3 (900 mg, 1.0 mmol) obtained in the same manner as in Synthesis Example 3, 4-cyanophenylboronic acid (441 mg, 3 mmol), potassium carbonate (1.38 g, 10 mmol) and tetrakis(triphenylphosphine)palladium(0) (70 mg, 0.1 mmol) were added to N-methyl-2-pyrrolidone (100 mL), and stirred at 130° C. for 24 hours. The mixture was cooled to room temperature, then poured into water (100 mL), and extracted with dichloromethane. The solvent was evaporated away under reduced pressure from the resultant organic layer, and the resultant crude product was purified by column chromatography using a mixed solvent of n-hexane/chloroform=2/1 (by volume) as an eluent to obtain a red solid of the intended product, Compound 5 at a yield of 670 mg, 71%.

¹H NMR (500 MHz, CDCl₃) δ 6.07 (d, J=10.0 Hz, 4H, Ar H), 6.59 (t, J=7.5 Hz, 4H, Ar H), 6.68 (t, J=10.0 Hz, 4H, Ar H), 6.75 (d, J=10.0 Hz, 4H, Ar H), 7.89 (t, J=10.0 Hz, 6H, Ar H), 7.99 (d, J=10.0 Hz, 4H, Ar H), 8.12 (d, J=10.0 Hz, 2H, Ar H), 8.60 (s, 2H, Ar H), 8.92 (s, 2H, Ar H), 9.77 (d, J=10.0 Hz, 2H, Ar H), 9.90 (d, J=10.0 Hz, 2H, Ar H);

¹³C NMR (125 MHz, CDCl₃, δ): 111.86, 113.33, 115.76, 118.73, 121.80, 123.33, 125.94, 127.22, 127.36, 128.23, 128.35, 129.04, 129.37, 130.24, 130.52, 131.02, 132.11, 132.93, 133.82, 134.17, 140.54, 140.79, 141.04, 141.09, 144.05, 145.31;

MALDI-TOF-MS: m/z: m/z: calculated for C₆₆H₃₆N₆O₂: 945.0540, found: 946.87.

### (Synthesis Example 6) Synthesis of Compound 6

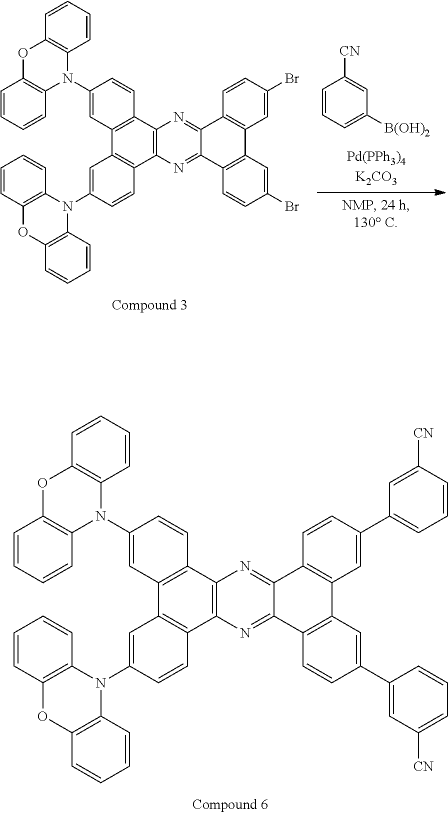

In a nitrogen atmosphere, Compound 3 (900 mg, 1.0 mmol) obtained in the same manner as in Synthesis Example 3, 3-cyanophenylboronic acid (441 mg, 3 mmol), potassium carbonate (1.38 g, 10 mmol) and tetrakis(triphenylphosphine)palladium(0) (70 mg, 0.1 mmol) were added to N-methyl-2-pyrrolidone (100 mL), and stirred at 130° C. for 24 hours. The mixture was cooled down to room temperature, poured into water (100 mL), and extracted with dichloromethane. The solvent was evaporated away under reduced pressure from the resultant organic layer, and the resultant crude product was purified by column chromatography using 1,2-dichlorobenzene as a n eluent to obtain a red solid of the intended product, Compound 6 at a yield of 560 mg, 59%.

MALDI-TOF-MS: m/z: calculated for C₆₆H₃₆N₆O₂: 945.0540, found: 946.77.

### (Synthesis Example 7) Synthesis of Compound 7

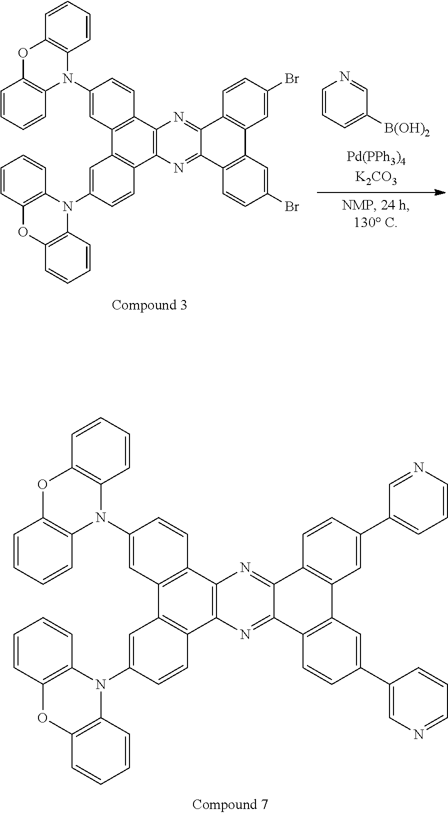

In a nitrogen atmosphere, Compound 3 (900 mg, 1.0 mmol) obtained in the same manner as in Synthesis Example 3, 3-pyrrolidineboronic acid (370 mg, 3 mmol), potassium carbonate (1.38 g, 10 mmol) and tetrakis(triphenylphosphine)palladium(0) (70 mg, 0.1 mmol) were added to N-methyl-2-pyrrolidone (100 mL), and stirred at 130° C. for 24 hours. The mixture was cooled down to room temperature, poured into water (100 mL), and extracted with dichloromethane. The solvent was evaporated away under reduced pressure from the resultant organic layer, and the resultant crude product was purified by column chromatography using a mixed solvent of n-hexane/chloroform=2/1 (by volume) as an eluent to obtain a red solid of the intended product, Compound 7 at a yield of 667 mg, 74%.

¹H NMR (500 MHz, CDCl₃) δ 6.08 (d, J=10.0 Hz, 4H, Ar H), 6.60 (t, J=7.5 Hz, 4H, Ar H), 6.68 (t, J=10.0 Hz, 4H, Ar H), 6.75 (d, J=10.0 Hz, 4H, Ar H), 7.54 (t, J=10.0 Hz, 2H, Ar H), 7.88 (d, J=10.0 Hz, 2H, Ar H), 8.12 (d, J=10.0 Hz, 2H, Ar H), 8.19 (d, J=10.0 Hz, 2H, Ar H), 8.60 (s, 2H, Ar H), 8.75 (d, J=5.0 Hz, 2H, Ar H), 8.94 (s, 2H, Ar H), 9.16(s, 2H, Ar H), 9.77 (d, J=5.0 Hz, 2H, Ar H), 9.91 (d, J=10.0 Hz, 2H, Ar H);

¹³C NMR cannot be measured due to poor solubility.

MALDI-TOF-MS: m/z: calculated for C₆₆H₃₆N₆O₂: 897.01. found: 898.17.

### (Synthesis Example 8) Synthesis of Compound 8

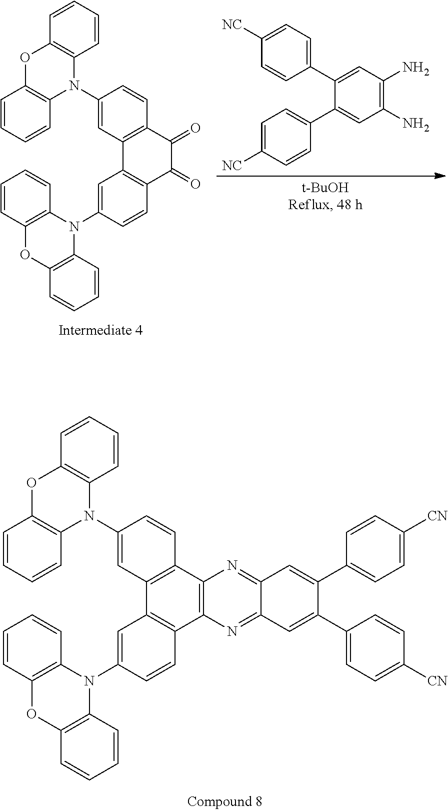 [SMILES: O=C1C(=O)c2ccc(N3c4ccccc4Oc4ccccc43)cc2-c2cc(N3c4ccccc4Oc4ccccc43)ccc21 ; N#Cc1ccc(-c2cc(N)c(N)cc2-c2ccc(C#N)cc2)cc1 ; N#Cc1ccc(-c2cc3nc4c5ccc(N6c7ccccc7Oc7ccccc76)cc5c5cc(N6c7ccccc7Oc7ccccc76)ccc5c4nc3cc2-c2ccc(C#N)cc2)cc1]

In a nitrogen atmosphere, Intermediate 4 (570 mg, 1.0 mmol) obtained in the same manner as in Synthesis Example 2, 4′,5′-diamino-[1,1′:2′1″-terphenyl]-4,4″-dicarbonitrile (375 mg, 1.2 mmol) were added to tert-butyl alcohol (100 mL), and stirred at 105° C. for 48 hours. The mixture was cooled down to room temperature, and mixed with ice with water. The precipitated solid was collected by filtration under reduced pressure, washed with methanol and purified by column chromatography. The purified faction was recrystallized with chloroform to obtain a deep yellow solid of the intended product, Compound 8 at a yield of 750 mg, 89%.

¹H NMR (500 MHz, CDCl₃) δ 6.04 (d, J=10.0 Hz, 4H, Ar H), 6.57 (t, J=7.5 Hz, 4H, Ar H), 6.67 (t, J=10.0 Hz, 4H, Ar H), 6.74 (d, J=10.0 Hz, 4H, Ar H), 7.44 (d, J=10.0 Hz, 4H, Ar H), 7.67 (d, J=10.0 Hz, 4H, Ar H), 7.80 (d, J=10.0 Hz, 2H, Ar H), 8.48 (s, 4H, Ar H), 9.67 (d, J=10.0 Hz, 2H, Ar H);

¹³C NMR (125 MHz, CDCl₃, δ): 111.89, 113.30, 115.79, 118.37, 121.87, 123.32, 125.96, 129.79, 129.99, 130.59, 131.31, 131.43, 132.29, 133.99, 134.26, 141.14, 141.80, 141.91, 143.06, 144.03, 144.28;

MALDI-TOF-MS: m/z: calculated for C₆₆H₃₆N₆O₂: 844.9340, found: 845.37.

### (Synthesis Example 9) Synthesis of Compound 9

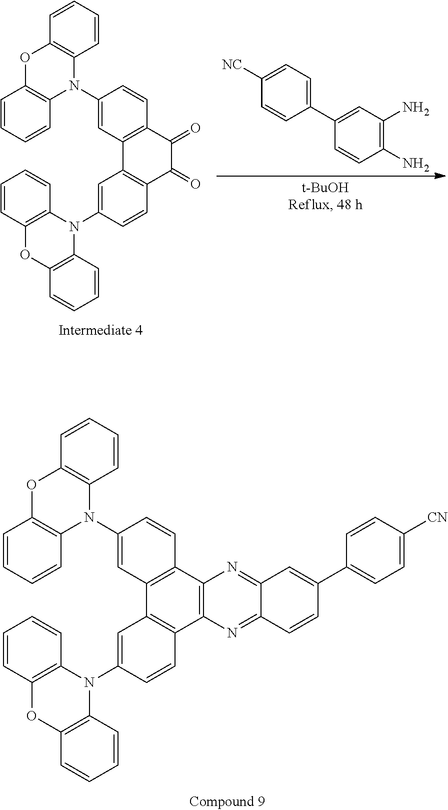 [SMILES: O=C1C(=O)c2ccc(N3c4ccccc4Oc4ccccc43)cc2-c2cc(N3c4ccccc4Oc4ccccc43)ccc21 ; N#Cc1ccc(-c2ccc(N)c(N)c2)cc1 ; N#Cc1ccc(-c2ccc3nc4c5ccc(N6c7ccccc7Oc7ccccc76)cc5c5cc(N6c7ccccc7Oc7ccccc76)ccc5c4nc3c2)cc1]

In a nitrogen atmosphere, Intermediate 4 (570 mg, 1.0 mmol) obtained in the same manner as in Synthesis Example 2, and 3′,4′-diamino-[1,1′-biphenyl]-4-carbonitrile (250 mg, 1.2 mmol) were added to tert-butyl alcohol (100 mL), and stirred at 105° C. for 48 hours. The mixture was cooled down to room temperature, and then mixed with water with ice. The precipitated solid was collected by filtration under reduced pressure, washed with methanol, and purified by column chromatography. The purified fraction was recrystallized with chloroform to obtain a deep yellow solid of the intended product, Compound 9 at a yield of 605 mg, 81%.

¹H NMR (500 MHz, CDCl₃) δ 6.04 (d, J=10.0 Hz, 4H, Ar H), 6.58 (t, J=7.5 Hz, 4H, Ar H), 6.67 (t, J=10.0 Hz, 4H, Ar H), 6.74 (d, J=10.0 Hz, 4H, Ar H), 7.80 (d, J=10.0 Hz, 2H, Ar H), 7.88 (d, J=10.0 Hz, 2H, Ar H), 8.80 (d, J=10.0 Hz, 2H, Ar H), 8.18 (d, J=10.0 Hz, 1H, Ar H), 9.50 (d, J=10.0 Hz, 3H, Ar H), 8.63 (s, 1H, Ar H), 9.69 (d, J=10.0 Hz, 2H, Ar H);

¹³C NMR (125 MHz, CDCl₃, δ): 113.32, 115.75, 121.81, 123.33, 125.91, 127.77, 128.27, 129.59, 129.78, 130.52, 131.19, 132.98, 134.06, 134.17, 140.91, 144.03;

MALDI-TOF-MS: m/z: m/z: calculated for C₅₁H₂₉N₅O₂: 743.8260, found: 744.08.

### (Synthesis Example 10) Synthesis of Compound 10

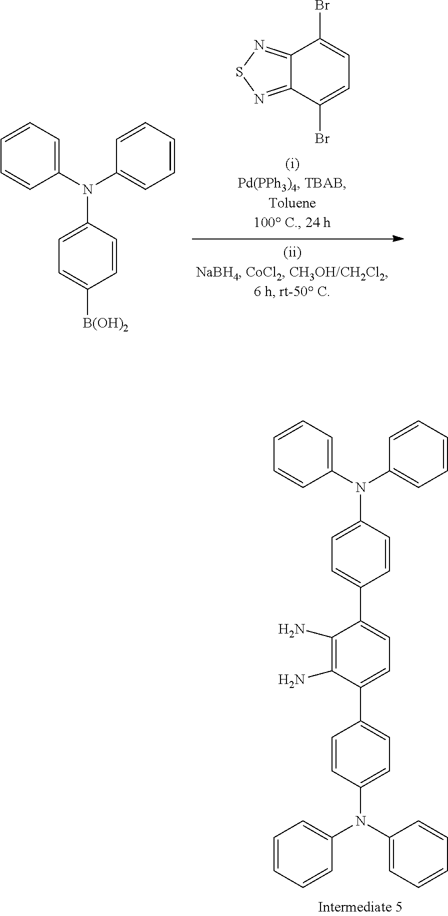 [SMILES: OB(O)c1ccc(N(c2ccccc2)c2ccccc2)cc1 ; Brc1ccc(Br)c2nsnc12 ; Nc1c(-c2ccc(N(c3ccccc3)c3ccccc3)cc2)ccc(-c2ccc(N(c3ccccc3)c3ccccc3)cc2)c1N]

In a nitrogen atmosphere, an aqueous solution of 2 M sodium hydroxide (24 mmol) was added to a solution prepared by dissolving 4,7-dibromobenzo[c][1,2,5]thiadiazole (0.59 g, 2 mmol), (4-(4-diphenylamino)phenyl)boronic acid (1.30 g, 4.5 mmol), tetrabutylammonium bromide (0.128 g, 0.4 mmol) and tetrakis(triphenylphosphine)palladium(0) (0.0578 g, 0.050 mmol) in toluene (40 mL), and stirred at 100° C. for 24 hours. An aqueous ammonium chloride solution was added to the reaction liquid to stop the reaction, then the reaction liquid was extracted with dichloromethane, and the resultant organic layer was dried with sodium sulfate, and the solvent was evaporated away under reduced pressure.

The resultant crude product was dissolved in dichloromethane (220 mL), and methanol (100 mL) was added thereto and stirred for 30 minutes. To the mixture, added were cobalt(II) chloride hexahydrate (0.237 g, 2 mmol), methanol (150 mL) and sodium borohydride (0.37 g, 10 mmol), and stirred for 6 hours at 50° C. Water was added to the reaction liquid to stop the reaction, then this was extracted with dichloromethane, the resultant organic layer was dried with anhydrous sodium sulfate, and the solvent was evaporated away under reduced pressure. The crude product was purified by column chromatography to obtain a pale black solid of Intermediate 5 (N4,N4,N4″,N4″-tetraphenyl-[1,1′:4′,1″-terphenyl]-2′,3′,4,4″-tetramine) at a yield of 0.39 g, 31%.

MS ASAP: m/z: calculated for C₄₆H₄₂N₄O₄: 594.76, found: 595.70.

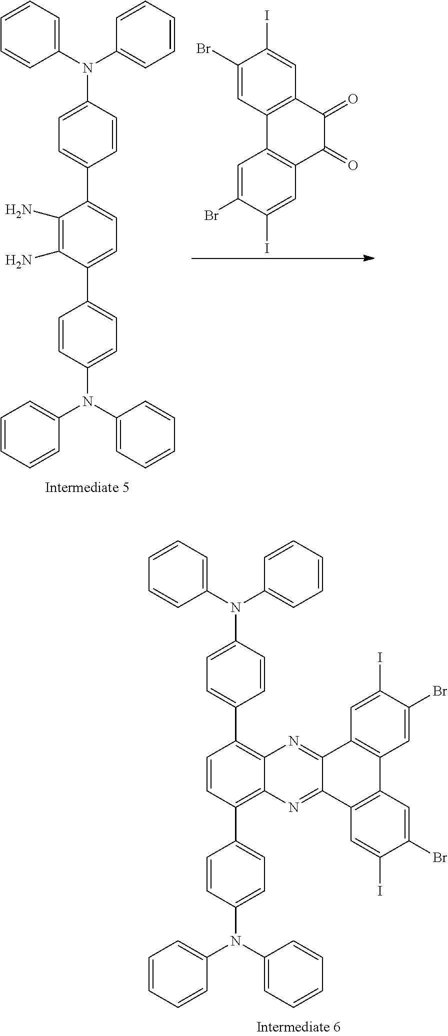

In a nitrogen atmosphere, Intermediate 5 (0.35 g, 0.6 mmol) and 3,6-dibromo-2,7-diiodophenanthrene-9,10-dione (0.37 g, 0.6 mmol) were added to acetic acid (100 mL), and stirred at 125° C. for 24 hours. The mixture was cooled down to room temperature, and then mixed with water with ice. The precipitated solid was collected by filtration under reduced pressure, washed with methanol and recrystallized with chloroform to obtain a deep red solid of Intermediate 6 (4,4′-(3,6-dibromo-2,7-diiododibenzo[a,c]phenazine-10,13-diyl)-bis(N,N-diphenylaniline)) at a yield of 0.360 g, 51%.

MALDI-TOF-MS: m/z: calculated for C₅₆H₃₄Br₂I₂N₄: 1176.53, found: 1177.27.

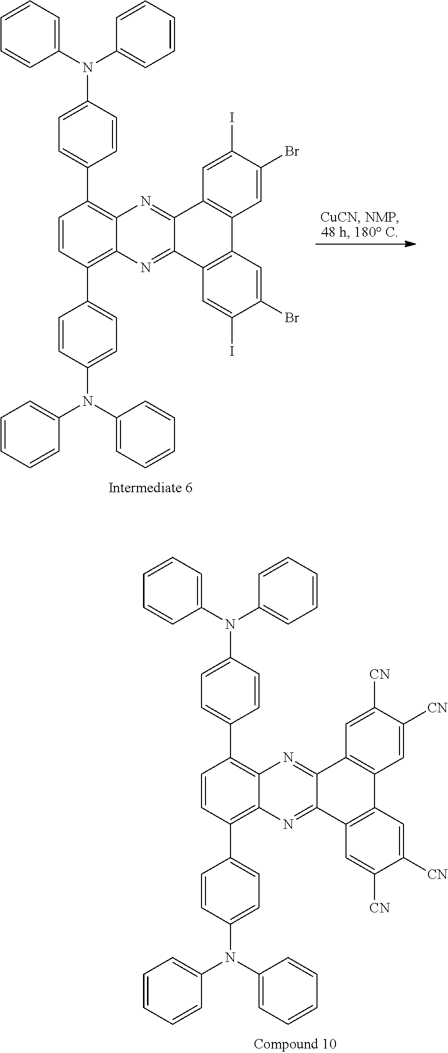 [SMILES: Brc1cc2c3cc(Br)c(I)cc3c3nc4c(-c5ccc(N(c6ccccc6)c6ccccc6)cc5)ccc(-c5ccc(N(c6ccccc6)c6ccccc6)cc5)c4nc3c2cc1I ; N#Cc1cc2c3cc(C#N)c(C#N)cc3c3nc4c(-c5ccc(N(c6ccccc6)c6ccccc6)cc5)ccc(-c5ccc(N(c6ccccc6)c6ccccc6)cc5)c4nc3c2cc1C#N]

In an argon atmosphere, Intermediate 6 (0.350 g, 0.297 mmol) and copper(I) cyanide (0.268 g, 3.0 mmol) were added to anhydrous N-methyl-2-pyrrolidone (150 mL), and heated at 180° C. for 48 hours. The mixture was cooled down to room temperature, then aqueous 2% ammonia (500 mL) was added thereto, and this was extracted three times with toluene (50 mL). The resultant organic layer was washed with water, and dried with magnesium sulfate. The crude product was purified by column chromatography using a mixed solvent of toluene/chloroform=9/1 (by volume) as an eluent to obtain a dark blue solid of the intended product, Compound 10 at a yield of 92 mg, 36%.

¹H NMR (500 MHz, CDCl₃, δ): 7.06 (m, 4H, Ar H), 7.30 (m, 20H, Ar H), 7.80 (d, J=5.0 Hz, 4H; Ar H), 7.99 (s, 2H, Ar H), 8.57 (s, 2H, Ar H), 9.50 (s, 2H, Ar H);

¹³C NMR (125 MHz, CDCl₃, δ): 113.06, 114.14, 114.26, 119.80, 121.89, 123.37, 127.72, 128.28, 129.64, 130.19, 130.83, 133.00, 136.17, 137.73, 139.79, 145.40, 146.50;

MALDITOF-MS: m/z: calculated for C₆₀H₃₄N₈: 866.9880, found: 867.46.

### (Synthesis Example 11) Synthesis of Compound 11

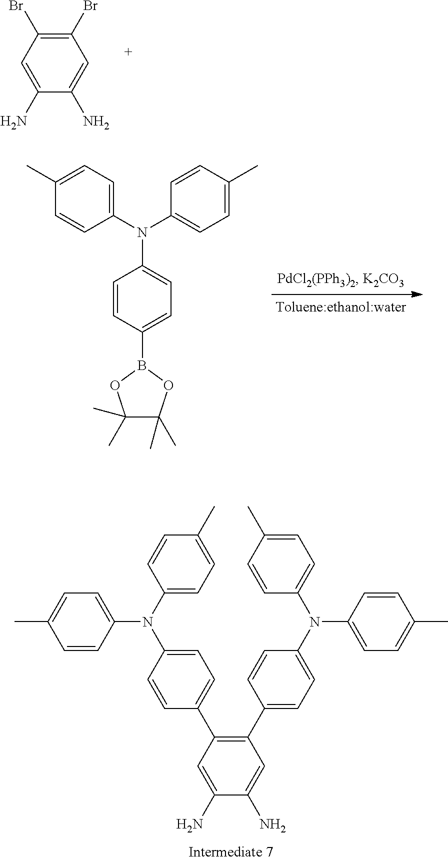

In a nitrogen atmosphere, 4,5-dibromobenzene-1,2-diamine (870 mg, 3.3 mmol), 4-methyl-N-(4-(4,4,5,5-tetramethyl-1,3,2-dioxabororan-2-yl)phenyl)-N-(p-tolyl)aniline (3.3 g, 8.26 mmol), potassium carbonate (2.28 g, 16.5 mmol) and bis(triphenylphosphine)palladium(II) dichloride (92.6 mg, 0.132 mmol) were added to a mixed solvent (150 mL) of toluene/ethanol/water=4/1/1 (by volume), and stirred at 90° C. for 24 hours. The mixture was cooled down to room temperature, poured into water (100 mL), and extracted with dichloromethane. The solvent was evaporated away under reduced pressure from the resultant organic layer, and the resultant crude product was purified by column chromatography using a mixed solvent of n-hexane/ethyl acetate=2/1 (by volume) as an eluent. By the process, a white solid of Intermediate 7 (N4,N4,N4″,N4″-tetra-p-tolyl-[1,1′:2′,1″-terphenyl]-4,4′,4″,5′-tetramine) was obtained at a yield of 1.75 g, 81%.

¹H NMR (500 MHz, DMSO-d⁶) δ 2.24 (s, 12H, methyl H), 4.58 (s, 4H, —NH₂), 6.55 (s, 2H, Ar H), 6.75 (d, J=10.0 Hz, 4H, Ar H), 6.84 (m, 12H, Ar H), 7.04 (t, J=10.0 Hz, 8H, Ar H).

MS ASAP: m/z: calculated for C₄₆H₄₂N₄: 650.87, found: 650.85

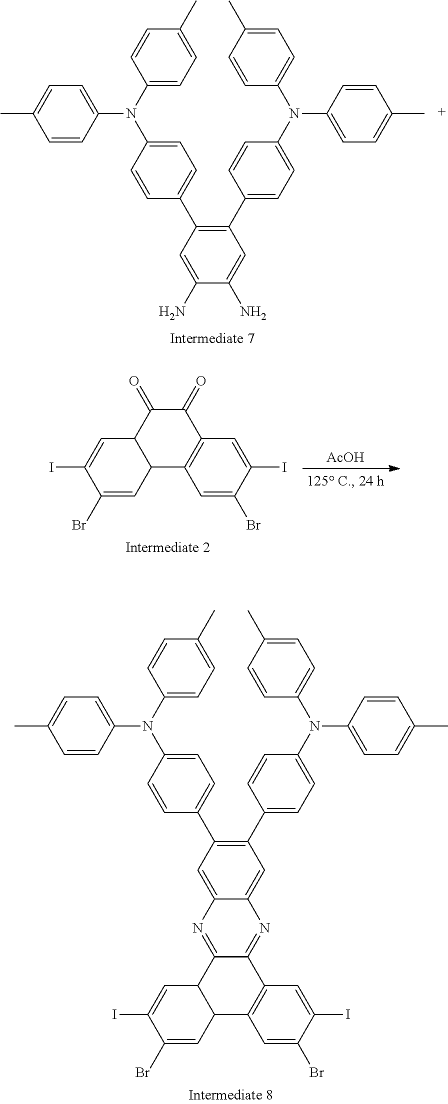

In a nitrogen atmosphere, Intermediate 7 (710 mg, 1.1 mmol) and Intermediate 2 (617 mg, 1.0 mmol) obtained in the same manner as in Synthesis Example 1 were added to acetic acid (250 mL), and stirred at 125° C. for 24 hours. The mixture was cooled down to room temperature, and then mixed with water with ice. The precipitated solid was collected by filtration under reduced pressure, washed with methanol and recrystallized with chloroform to obtain a deep red solid of Intermediate 8 (4,4′-(3,6-dibromo-2,7-diiododibenzo[a,c]phenazine-11,12-diyl)-bis(N,N-di-p-tolylaniline)) at a yield of 1.05 g, 85%.

¹H NMR (500 MHz, CDCl₃) δ 2.34 (s, 12H, methyl H), 6.97 (d, J=10.0 Hz, 4H, Ar H), 7.09 (m, 18H, Ar H), 7.16 (d, J=10.0 Hz, 4H, Ar H), 8.15 (s, 1H, ArH), 8.28 (s, 2H, ArH), 8.59 (d, J=10.0 Hz, 3H, Ar H), 9.74 (s, 2H, Ar H).

MALDI-TOF-MS: m/z: calculated for C₆₀H₄₂Br₂I₂N₄: 1232.64, found: 1232.44.

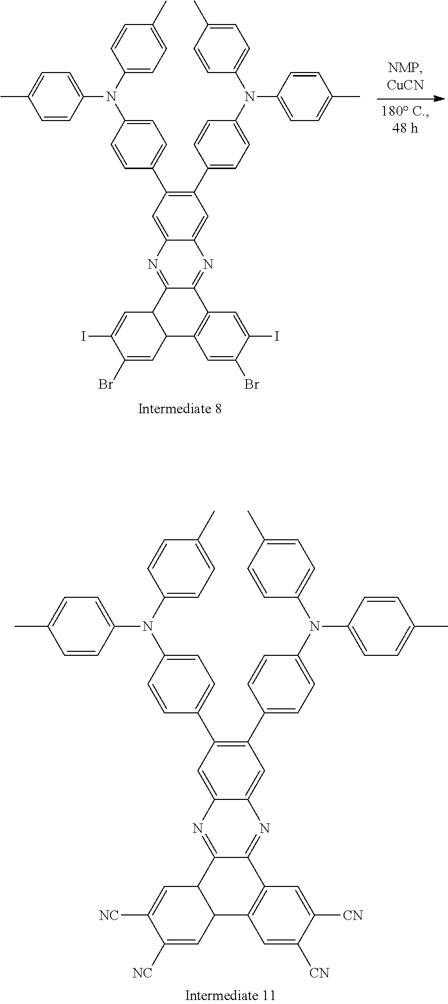

In an argon atmosphere, Intermediate 8 (950 mg, 0.77 mmol) and copper(I) cyanide (690 mg, 7.7 mmol) were added to anhydrous N-methyl-2-pyrrolidone (150 mL), and heated at 180° C. for 48 hours. The mixture was cooled down to room temperature, and then aqueous 2% ammonia (500 mL) was added thereto, and this was extracted three times with toluene (50 mL). The resultant organic layer was washed with water, and dried with magnesium sulfate. The crude product was purified by column chromatography using a mixed solvent of toluene/chloroform=9/1 (by volume) as an eluent to obtain a deep blue solid of the intended product, Compound 11 at a yield of 575 mg, 80%.

¹H NMR (500 MHz, CD₂Cl₂, δ): 2.33 (s, 12H, methyl H), 6.96 (d, J=10.0 Hz, 4H, Ar H), 7.03 (d, J=10.0 Hz, 8H, Ar H), 7.12 (d, J=10.0 Hz, 8H, Ar H), 7.19 (d, J=10.0 Hz, 4H, Ar H), 8.40 (s, 2H, Ar H), 9.02 (s, 2H, Ar H), 9.67 (s, 2H, Ar H);

¹³C NMR (125 MHz, CD₂Cl₂, δ): 20.55, 115.30, 115.66, 115.81, 120.88, 125.12, 129.38, 129.99, 130.63, 130.71, 131.41, 133.37, 134.86, 139.39, 143.00, 144.82, 148.22, 149.25, 150.27;

MALDITOF-MS: m/z: calculated for C₆₄H₄₂N₈: 923.09, found: 923.81.

### (Synthesis Example 12) Synthesis of Compound 12

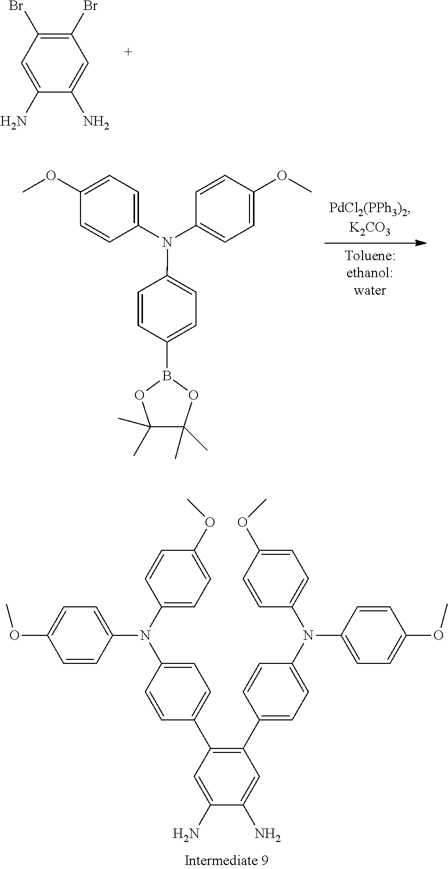

In a nitrogen atmosphere, 4,5-dibromobenzene-1,2-diamine (870 mg, 3.3 mmol), 4-methoxy-N-(4-methoxyphenyl)-N-(4-(4,4,5,5-tetramethyl-1,3,2-dioxabororan-2-yl)phenyl)aniline (3.20 g, 7.2 mmol), potassium carbonate (2.28 g, 16.5 mmol) and bis(triphenylphosphine)palladium(II) dichloride (92.6 mg, 0.132 mmol) were added to a mixed solvent (150 mL) of toluene/ethanol/water=4/1/1 (by volume), and stirred at 90° C. for 24 hours. The mixture was cooled to room temperature, poured into water (100 mL), and extracted with dichloromethane. The solvent was evaporated away under reduced pressure from the resultant organic layer, and the resultant crude product was purified by column chromatography using a mixed solvent of n-hexane/ethyl acetate=2/1 (by volume) as an eluent. By the process, a white sold of Intermediate 9 (N4,N4,N4″,N4″-tetrakis(4-methoxyphenyl)-[1,1′:2′,1″-tetraphenyl]-4,4′,4″,5′-tetramine) was obtained at a yield of 2.20 g, 93%.

¹H NMR (500 MHz, DMSO-d⁶) δ 3.71 (s, 12H, Methoxy H), 4.54 (s, 4H, —NH₂) 6.52 (s, 2H, Ar H), 6.64 (d, J=10.0 Hz, 4H, Ar H), 6.85 (m, 12H, Ar H), 6.95 (d, J=10.0 Hz, 8H, Ar H);

MS ASAP: m/z: calculated for C₄₆H₄₂N₄O₄: 714.86, found: 714.56.

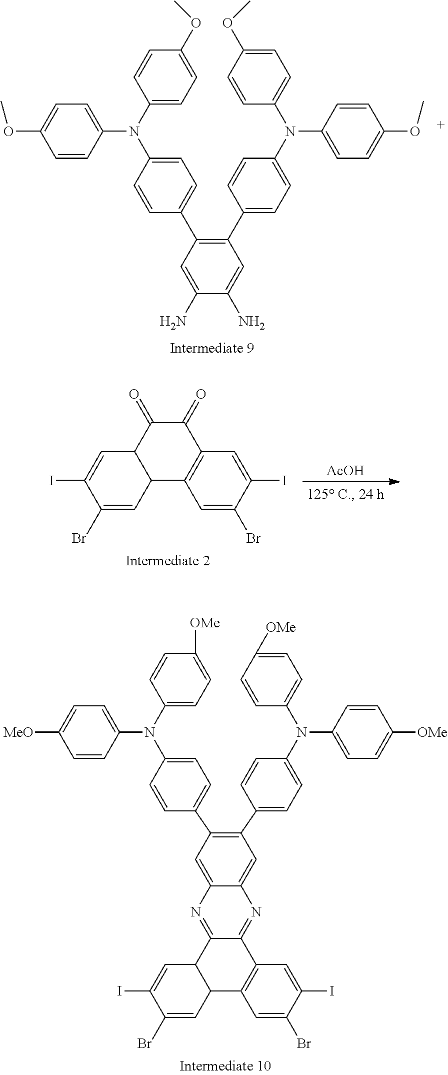

In a nitrogen atmosphere, Intermediate 9 (750 mg, 1.1 mmol) and Intermediate 2 (617 mg, 1.0 mmol) obtained in the same manner as in Synthesis Example 1 were added to acetic acid (100 mL), and stirred at 125° C. for 24 hours. The mixture was cooled down to room temperature, and then mixed with water with ice. The precipitated solid was collected by filtration under reduced pressure, washed with methanol and recrystallized with chloroform to obtain a deep red solid of Intermediate 10 (4,4′-(3,6-dibromo-2,7-diiododibenzo[a,c]phenazine-11,12-diyl)-bis(N,N-bis(4-methoxyphenyl)aniline) at a yield of 1.21 g, 94%.

¹H NMR (500 MHz, CDCl₃) δ 3.81 (s, 12H, methoxy H), 6.88 (d, J=10.0 Hz, 6H, Ar H), 6.89 (d, J=10.0 Hz, 6H, Ar H), 7.14 (m, 12H, Ar H), 8.25 (s, 2H, ArH), 8.57 (s, 2H, ArH), 9.71 (s, 2H, Ar H).

MALDI-TOF-MS: m/z: calculated for C₆₀H₄₂Br₂I₂N₄O₄: 1296.63, found: 1298.72.

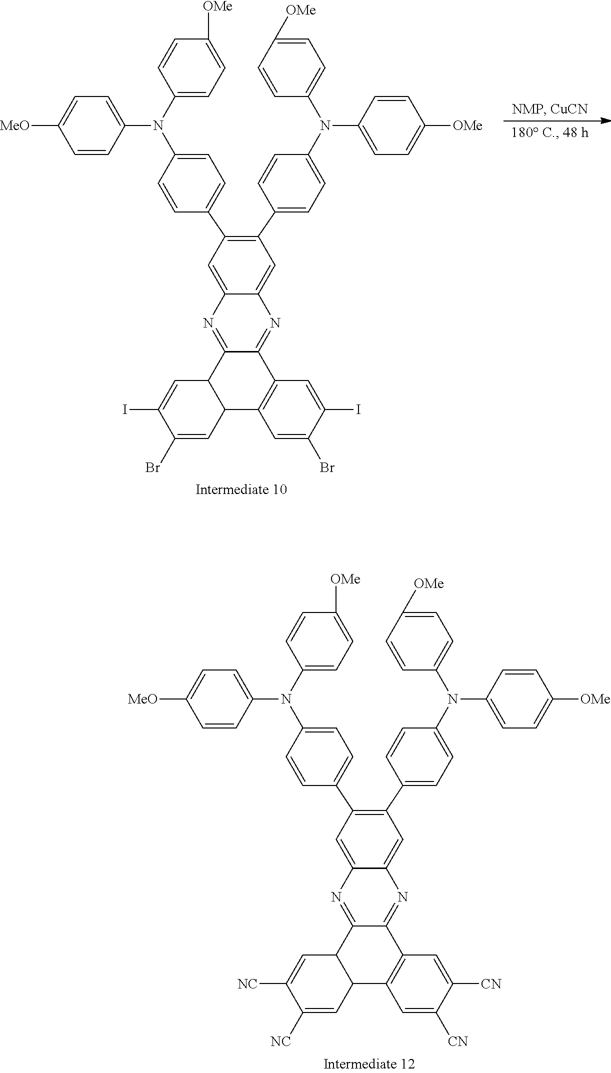 [SMILES: COc1ccc(N(c2ccc(OC)cc2)c2ccc(-c3cc4nc5c(nc4cc3-c3ccc(N(c4ccc(OC)cc4)c4ccc(OC)cc4)cc3)C3C=C(I)C(Br)=CC3c3cc(Br)c(I)cc3-5)cc2)cc1 ; COc1ccc(N(c2ccc(OC)cc2)c2ccc(-c3cc4nc5c(nc4cc3-c3ccc(N(c4ccc(OC)cc4)c4ccc(OC)cc4)cc3)C3C=C(C#N)C(C#N)=CC3c3cc(C#N)c(C#N)cc3-5)cc2)cc1]

In an argon atmosphere, Intermediate 10 (925 mg, 0.71 mmol) and copper (I) cyanide (640 mg, 7.1 mmol) were added to anhydrous N-methyl-2-pyrrolidone (100 mL), and heated at 180° C. for 48 hours. The mixture was cooled down to room temperature, then aqueous 2% ammonia (500 mL) was added thereto, and this was extracted three times with toluene (50 mL). The resultant organic layer was washed with water, and dried with magnesium sulfate. The crude product was purified by column chromatography using a mixed solvent of toluene/chloroform=9/1 (by volume) as an eluent to obtain a deep blue solid of the intended product, Compound 12 at a yield of 550 mg, 78%.

¹H NMR (500 MHz, CD₂Cl₂, δ): 3.84 (s, 12H, methoxy H), 6.91 (t, J=10.0 Hz, 12H, Ar H), 7.15 (d, J=10.0 Hz, 8H, Ar H), 7.20 (d, J=10.0 Hz, 4H, Ar H), 8.39 (s, 2H, Ar H), 9.03 (s, 2H, Ar H), 9.97 (s, 2H, Ar H);

¹³C NMR (125 MHz, CD₂Cl₂, δ): 55.45, 114.76, 115.30, 115.33, 115.62, 115.73, 118.94, 127.06, 129.27, 129.90, 130.56, 131.05, 131.34, 132.36, 134.84, 139.27, 140.31, 142.98, 147.22, 148.77, 156.45;

MALDITOF-MS: m/z: calculated for C₆₄H₄₂N₈O₄: 987.09, found: 988.01.

### (Evaluation of Physical Properties)

Compound I synthesized was subjected to thermogravimetric differential thermal analysis and was confirmed to have a high decomposition temperature of 562° C. and have high thermal stability. As a result of cyclic voltammetry, Compound 1 was confirmed to be electrochemically stable.

### (Example 1) Production and Evaluation of Organic Photoluminescent Device Using Compound 1

In this Example, an organic photoluminescent device (PL device) was produced using Compound 1, and evaluated for the following items.

### Evaluation of Emission Characteristics of Solution and Thin Film of Compound 1

In a glove box of an Ar atmosphere, a toluene solution of Compound 1 (concentration 10⁻⁵ M) was prepared.

In addition, Compound 1 and mCBP were evaporated from different evaporation sources on a quartz substrate according to a vacuum evaporation method under the condition of a vacuum degree of 5×10⁻⁴ Pa or less to form a thin film (mixed film 1) where the concentration of Compound 1 was 1% by weight, and a thin film (mixed film 2) where the concentration of Compound 1 was 10% by weight, each in a thickness of 100 nm thereby producing organic photoluminescent devices.

The toluene solution of Compound 1 and the mixed films 1 and 2 of Compound 1 and mCBP were measured for emission characteristics, and the results are shown in Table 1. In Table 1, “k_{RISC}” indicates a reverse intersystem crossing rate constant. In Table 1 and Tables 2 to 4 below, “-” indicates that the characteristic value were not measured.

TABLE 1

| | Emission | PL Quantum Yield | | |
|---|---|---|---|---|
| | Maximum | Instantaneous | Delayed | |
| | Wavelength | Fluorescence | Fluorescence | k_{RISC} |
| Kind of Device | [nm] | [%] | [%] | [10⁵ s⁻¹] |
| Toluene Solution of Compound 1 (10⁻⁵ M) | 674 | 53.7 | 24.0 | 0.8 |
| Mixed Film 1 of Compound 1 (1 wt. %) | 672 | 32.5 | 45.6 | 1.1 |
| Mixed Film 1 of Compound 1 (10 wt. %) | 729 | 19.1 | 21.7 | 0.8 |

As shown in Table 1, the toluene solution of Compound 1 and mixed films 1 and 2 thereof all have a high photoluminescence quantum yield (PL quantum yield), and have extremely high k_{RISC}. These confirm that Compound 1 is a compound capable of readily undergoing reverse intersystem crossing from triplet to singlet. It is suggested that the reason why the compound has such a high PL quantum yield is because owing to such reverse intersystem crossing, the excited triplet energy is converted into excited singlet energy and used for light emission. In addition, also the thin film 2 where the concentration of Compound 1 is 10% by weight may have a high PL quantum yield of 40.8%, from which it is suggested that Compound 1 is a luminescent molecule that hardly undergoes aggregation induced extinction. Further, the emission maximum wavelength of the mixed film 2 is shifted toward the long wavelength side than the emission maximum wavelength of the mixed film 1 where the concentration of Compound 1 is 1% by weight, and from this, it is known that by varying the concentration of Compound 1, the emission wavelength of the mixed film may be controlled.

### Measurement of Concentration Dependency of Emission Peak

Mixed films were formed in the same manner as that of the film formation method for the mixed film 1, except that the concentration of Compound 1 was varied within a range of 0.5 to 100% by weight (wt %), thereby producing organic photoluminescent devices (PL devices).

Emission spectra of the thus-produced mixed films are shown in FIG. 2. In FIG. 2, the numeral value with a unit of “wt %” indicates the concentration of Compound 1 in the mixed films.

As shown in FIG. 2, the emission peaks observed from the mixed films shifted toward the long wavelength side with the increase in the concentration of Compound 1. From this, it is known that, of the light emitting devices using Compound 1, the emission peak may be readily controlled within a range of a broad wavelength range of 590 to 990 nm, by a simple method of varying the concentration of Compound 1 in the devices.

Evaluation of Properties of Thin Films Added with BBTDTPA

According to a vacuum evaporation method, BBTDTPA, Compound 1 and mCBP were evaporated on a quartz substrate from different evaporation sources under the condition of a vacuum degree of 5×10⁻⁴ Pa or less to form a thin film (mixed film 3), thereby producing an organic photoluminescent device. At that time, the concentration of BBTDTPA was 1% by weight, the concentration of Compound 1 was 10% by weight.

The emission spectrum of the thus-formed mixed film 3 was measured, and the emission maximum wavelength was 874 nm and the PL quantum yield was 12.9%. The emission maximum wavelength of the film was nearly the same as that of BBTDTPA, from which it is known that the light emission of the mixed film 3 was derived from BBTDTPA. In addition, the photoabsorption spectrum of a toluene solution of BBTDTPA was measured, and compared with the emission spectrum of the mixed film 2 (a thin film not containing BBTDTPA, and the concentration of Compound 1 therein was 10% by weight) and, as a result, the emission range of the mixed film 2 sufficiently overlapped with the absorption range of the BBTDTPA solution. These results suggest that the emission from the mixed film 3 results from a series of processes where the excited single energy of Compound 1 transferred to BBTDTPA by a Foerster transfer mechanism and BBTDTPA excited by the excited singlet energy emitted light. From this, it is confirmed that Compound 1 may function as an assist dopant that contributes toward light emission of BBTDTPA.

### (Example 2) Production and Evaluation of Organic Electroluminescent Device Using a Mixed Film of Compound 1 and mCBP as a Light Emitting Layer

On a glass substrate having, as formed thereon, an anode of indium tin oxide (ITO) having a thickness of 100 nm, the following thin films were laminated at a vacuum degree of 5.0×10⁻⁴ Pa according to a vacuum evaporation method. First, HATCN was deposited on ITO at a thickness of 10 nm, then TAPC was deposited thereon at a thickness of 20 nm. Next, Compound 1 and mCBP were co-evaporated from different evaporation sources to form a layer having a thickness of 60 nm to be a light emitting layer. At that time, the concentration of Compound 1 was 10% by weight. Next, T2T was formed at a thickness of 10 nm, and BPyTP2 was formed thereon at a thickness of 50 nm. Further, Liq was formed at a thickness of 2 mm, and aluminum (Al) was deposited thereon at a thickness of 100 nm to be a cathode, thereby producing an organic electroluminescent device (EL device 1).

### (Example 3) Production and Evaluation of Organic Electroluminescent Device Using a Mixed Film of BBTDTPA, Compound 1 and mCBP as a Light Emitting Layer

An organic electroluminescent device (EL device 2) was produced in the same manner as in Example 2 except that the light emitting layer was formed by co-evaporation using three evaporation sources of BBTDTPA, Compound 1 and mCBP. At that time, the concentration of BBTDTPA was 1% by weight, and the concentration of Compound 1 was 10% by weight.

Device characteristics of the EL devices produced in Examples 1 and 2 are shown in Table 2. In Table 2, “LT95” indicates the time taken until the luminance reached 95% of the initial luminance.

TABLE 2

| | | Emission | Maximum | |
|---|---|---|---|---|
| | | Maximum | External Quantum | |
| | | Wavelength | Efficiency | LT95 |
| | Composition of Light Emitting Layer | [nm] | EQEₘₐₓ [%] | [hrs] |
| EL Device 1 | Compound 1 (10 wt %):mCBP (90 wt %) | 734 | 13.4 | 168 |
| EL Device 2 | BBTDTPA (1 wt %):Compound 1 (10 | 901 | 1.1 | >600 |
| | wt %):mCBP (89 wt %) | | | |

As shown in Table 2, the emission maximum wavelength λmax of EL devices 1 and 2 is 734 nm and 901 nm, respectively, each corresponding to the absorption wavelength of hemoglobin and the absorption wavelength of hemoglobin oxide. From this, it is known that EL devices 1 and 2 may be effectively utilized as a light source for pulse oximeter for measurement of the amount of oxygen in blood.

### (Examples 4 to 11) Production and Evaluation of Organic Photoluminescent Device Using Compounds 2 to 9

In a glove box of an Ar atmosphere, toluene solutions of Compounds 2 to 9 (concentration 10⁻⁵ M) were prepared respectively.

According to a vacuum evaporation method, any one of Compound 2 to 9 and CBP were evaporated on a quartz substrate from different evaporation sources under the condition of a vacuum degree of 5×10⁻⁴ Pa or less to form thin films (mixed films 4 to 11) each at a thickness 100 nm, thereby producing organic photoluminescent devices. At that time, the concentration of Compounds 2 to 9 in each mixed film was 5% by weight.

Further, other thin films (mixed films 12 to 19) were formed each at a thickness of 100 nm, in which the concentration of Compounds 2 to 9 was 5% by weight, according to the same film formation method as that for the mixed films 4 to 11 except that mCP was used in place of CBP, thereby producing organic photoluminescent devices.

The results of measurement of the emission characteristics of each toluene solution are shown in Table 3, and the results of measurement of the emission characteristics of each mixed film are shown in Table 4. As the PL quantum yield of the toluene solution, shown are both the value of the toluene solution measured in the air, and the value of the toluene solution purged with argon.

TABLE 3

| | Emission Maximum | PL Quantum Yield [%] | | Delayed Fluorescence |
|---|---|---|---|---|
| Compound in | Wavelength | | purged | Lifetime |
| Toluene Solution | [nm] | In the air | with Ar | [μs] |
| Compound 2 | 596 | 22 | 88 | 3.1 |
| Compound 3 | 607 | 17 | 60 | 1.8 |
| Compound 4 | 691 | 10 | 13 | — |
| Compound 5 | 640 | 24 | 48 | 1.6 |
| Compound 6 | 630 | 23 | 63 | 1.7 |
| Compound 7 | 619 | 23 | 63 | 2.2 |
| Compound 8 | 701 | 6 | 8 | — |
| Compound 9 | 635 | 18 | 41 | 1.6 |

TABLE 4

| | | | Emission | PL | | Delayed |
|---|---|---|---|---|---|---|
| | Component of Mixed Film | | Maximum | Quantum | | Fluorescence |
| Mixed | Light Emitting | Host | Wavelength | Yield | ΔE_{ST} | Lifetime |
| Film No | Material | Material | [nm] | [%] | [eV] | [μs] |
| Mixed Film 4 | Compound 2 | CBP | 563 | 94 | 0.04 | 1.7 |
| Mixed Film 5 | Compound 3 | CBP | — | — | — | — |
| Mixed Film 6 | Compound 4 | CBP | 637 | 38 | 0.04 | 1.2 |
| Mixed Film 7 | Compound 5 | CBP | 622 | 58 | 0.03 | 1.1 |
| Mixed Film 8 | Compound 6 | CBP | — | — | — | — |
| Mixed Film 9 | Compound 7 | CBP | — | — | — | — |
| Mixed Film 10 | Compound 8 | CBP | — | — | 0.06 | — |
| Mixed Film 11 | Compound 9 | CBP | 603 | 74 | 0.01 | — |
| Mixed Film 12 | Compound 2 | mCP | 560 | 100 | 0.04 | 2.3 |
| Mixed Film 13 | Compound 3 | mCP | — | — | — | — |
| Mixed Film 14 | Compound 4 | mCP | 626 | 57 | 0.04 | — |
| Mixed Film 15 | Compound 5 | mCP | 617 | 84 | 0.03 | 1.5 |
| Mixed Film 16 | Compound 6 | mCP | — | — | — | — |
| Mixed Film 17 | Compound 7 | mCP | — | — | — | — |
| Mixed Film 18 | Compound 8 | mCP | 635 | 48 | 0.06 | 2.0 |
| Mixed Film 19 | Compound 9 | mCP | 601 | 84 | 0.01 | 1.6 |

### (Example 12) Production of Organic Electroluminescent Device Using Mixed Film of Compound 2 and mCP as a Light Emitting Layer

On a glass substrate having, as formed thereon, an anode of indium tin oxide (ITO) having a thickness of 100 nm, the following thin films were laminated at a vacuum degree of 5.0×10⁻⁴ Pa according to a vacuum evaporation method. First, HATCN was deposited on ITO at a thickness of 10 nm, then TAPC was deposited thereon at a thickness of 40 nm. Next, TCTA was formed at a thickness of 10 nm. Next, Compound 2 and mCBP were co-evaporated from different evaporation sources to form a layer having a thickness of 20 nm to be a light emitting layer. At that time, the concentration of Compound 2 was 10% by weight. Next, TmPyPb was formed at a thickness of 655 nm. Further, Liq was formed at a thickness of 2 mm, and aluminum (Al) was deposited thereon at a thickness of 100 nm to be a cathode, thereby producing an organic electroluminescent device (EL device 3).

### (Examples 13 to 15) Production of Organic Electroluminescent Device Using Mixed Film of Compound 2 and Various Host Materials as a Light Emitting Layer

Other organic electroluminescent devices (EL devices 4 to 6) were produced in the same manner as in Example 12 except that mCBP, ID5 or CBP was used in place of mCP.

### (Examples 16 to 43) Production of Organic Electroluminescent Device Using Mixed Film of Compounds 2 to 9 and Various Host Materials as a Light Emitting Layer

Other organic electroluminescent devices (EL devices 7 to 34) were produced in the same manner as in Examples 12 to 15 except that any of Compounds 3 to 9 was used in place of Compound 2.

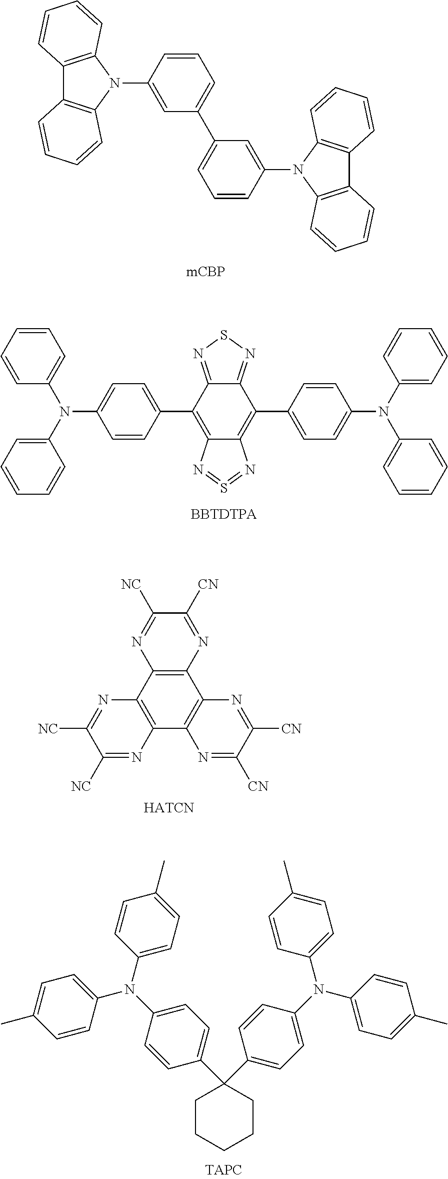

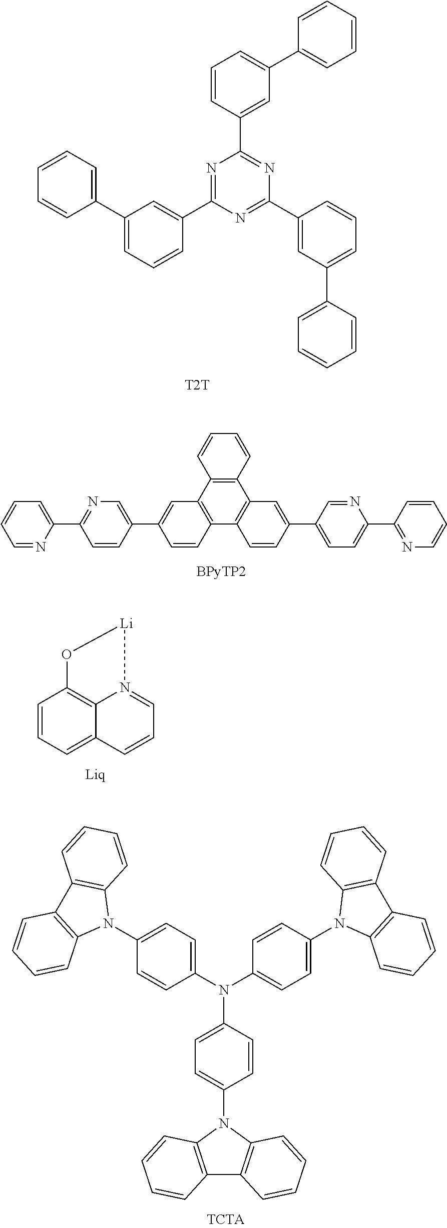

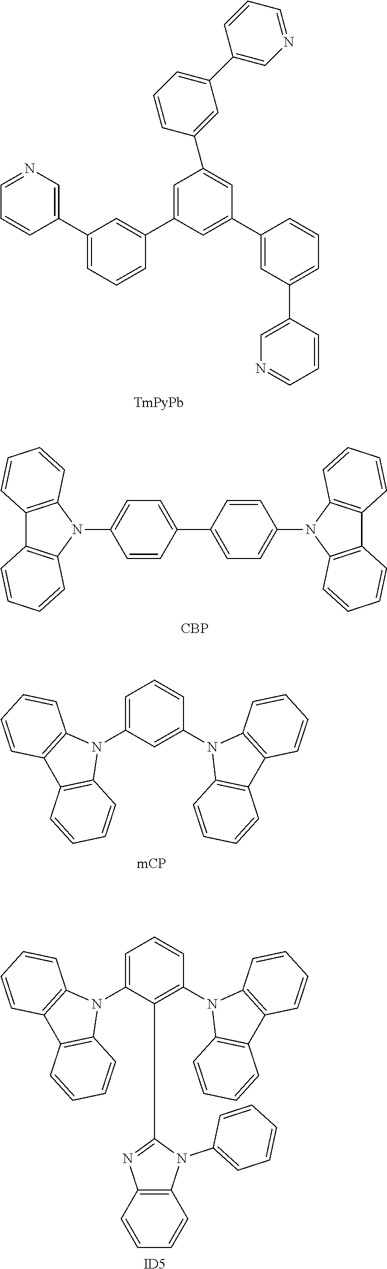

### INDUSTRIAL APPLICABILITY

According to the present invention, there may be provided a high-efficiency near-infrared emitting organic EL device. The organic EL device may be used for various applications such as night vision displays, optical communications, information protection devices, and healthcare devices. Consequently, the present invention possesses high industrial applicability.

1. A compound represented by the following general formula (1): 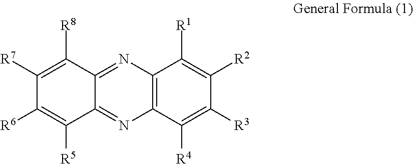
wherein R¹ to R⁸ each independently represent a hydrogen atom or a substituent, R¹ and R², R² and R³, R³ and R⁴, R⁵ and R⁶, R⁶ and R⁷, and R⁷ and R⁸ each may bond to each other to form a cyclic structure, but do not form a heteroaryl ring, and the general formula (1) satisfies at least one condition of the following (A) to (D):
(A) At least one of R¹ to R⁴ is *-Ar-D;
(B) R¹ and R² bond to each other to form an aromatic ring, and at least one *-Ar-D or D bonds to the aromatic ring;
(C) R² and R³ bond to each other to form an aromatic ring, and at least one *-Ar-D or D bonds to the aromatic ring; and
(D) R³ and R⁴ bond to each other to form an aromatic ring, and at least one *-Ar-D or D bonds to the aromatic ring,
wherein D represents a donor group, Ar represents an arylene group and * indicates a bonding position.

2. The compound according to claim 1, wherein two or more D's contained in (A) to (D) exist in a molecule of the compound.

3. The compound according to claim 2, which satisfies the (A) in which at least two of R¹ to R⁴ are *-Ar-D.

4. The compound according to claim 2, which satisfies (B) and (D).

5. The compound according to claim 1, wherein D's existing in a molecule of the compound all have the same structure.

6. The compound according to claim 1, wherein the general formula (1) satisfies at least one condition of the following (E) to (H):
(E) At least one of R⁵ to R⁸ is *-Ar-A;
(F) R⁵ and R⁶ bond to each other to form an aromatic ring, and at least one *-Ar-A or A bonds to the aromatic ring;
(G) R⁶ and R⁷ bond to each other to form an aromatic ring, and at least one *-Ar-A or A bonds to the aromatic ring; and
(H) R⁷ and R⁸ bond to each other to form an aromatic ring, and at least one *-Ar-A or A bonds to the aromatic ring,
wherein A represents an acceptor group, Ar represents an arylene group and * indicates a bonding position.

7. The compound according to claim 1, which is represented by the following general formula (2): 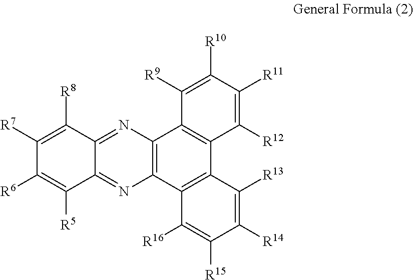
wherein R⁵ to R¹⁶ each independently represent a hydrogen atom or a substituent, R⁵ and R⁶, R⁶ and R⁷, R⁷ and R⁸, R⁹ and R¹⁰, R¹⁰ and R¹¹, R¹¹ and R¹², R¹² and R¹³, R¹³ and R¹⁴, R¹⁴ and R¹⁵, and R¹⁵ and R¹⁶ each may bond to each other to form a cyclic structure, but do not form a heteroaryl ring, and at least one of R⁹ to R¹⁶ is *-Ar-D or D, wherein D represents a donor group, Ar represents an arylene group, and * indicates a bonding position.

8. The compound according to claim 7, wherein at least one of R⁶ and R⁷ is *-Ar-A or A, A represents an acceptor group, Ar represents an arylene group, and * indicates a bonding position.

9. The compound according to claim 1, which is represented by the following general formula (3): 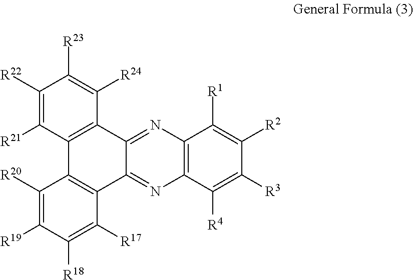
wherein R¹ to R⁴ and R¹⁷ to R²⁴ each independently represent a hydrogen atom or a substituent, R¹ and R², R² and R³, R³ and R⁴, R¹⁷ and R¹⁸, R¹⁸ and R¹⁹, R¹⁹ and R²⁰, R²⁰ and R²¹, R²¹ and R²², R²² and R²³, and R²³ and R²⁴ each may bond to each other to form a cyclic structure, and the general formula (3) satisfies at least one condition of the following (A) to (D):
(A) At least one of R¹ to R⁴ is *-Ar-D;
(B) R¹ and R² bond to each other to form an aromatic ring, and at least one *-Ar-D or D bonds to the aromatic ring;
(C) R² and R³ bond to each other to form an aromatic ring, and at least one *-Ar-D or D bond to the aromatic ring; and
(D) R³ and R⁴ bond to each other to form an aromatic ring, and at least one *-Ar-D or D bonds to the aromatic ring,
wherein D represents a donor group, Ar represents an arylene group and * indicates a bonding position.

10. The compound according to claim 1, which is represented by the following general formula (4): 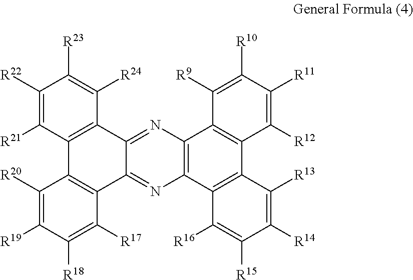
wherein R⁹ to R²⁴ each independently represent a hydrogen atom or a substituent, R⁹ and R¹⁰, R¹⁰ and R¹¹, R¹¹ and R¹², R¹² and R¹³, R¹³ and R¹⁴, R¹⁴ and R¹⁵, R¹⁵ and R¹⁶, R¹⁷ and R¹⁸, R¹⁸ and R¹⁹, R¹⁹ and R²⁰, R²⁰ and R²¹, R²¹ and R²², R²² and R²³, and R²³ and R²⁴ each may bond to each other to form a cyclic structure, and at least one of R⁹ to R¹⁶ is *-Ar-D or D, wherein D represents a donor group, Ar represents an arylene group, and * indicates a bonding position.

11. The compound according to claim 9, wherein at least one of R¹⁷ to R²⁴ is *-Ar-A or A, A represents an acceptor group, and * indicates a bonding position.

12. The compound according to claim 9, wherein at least one of R¹⁹ and R²² is *-Ar-A or A, A represents an acceptor group, and * indicates a bonding position.

13. The compound according to claim 7, wherein at least one of R¹¹ and R¹⁴ is *-Ar-D or D.

14-15. (canceled)

16. An organic light emitting device containing the compound of claim 1.

17. The organic light emitting device according to claim 16, containing the compound in a light emitting layer.

18. The organic light emitting device according to claim 17, wherein the light emitting layer contains a light emitting material, and among the light emission from the organic light emitting device, the amount of light emission from the light emitting material is the maximum.

19. The organic light emitting device according to claim 17, wherein the light emitting layer contains a host material.

20. The organic light emitting device according claim 16, having an emission peak wavelength at 590 to 990 nm.
